SEQUENCE LISTING (8 sequences)

SEQ ID NO: 1 - DNA, 1607 bp, Human
  CDS (126)...(1079)
  polyA_signal (1576)...(1582)
  polyA_site (1598)...(1607)
cgggggtctccatcctggaccaggacctggactacctgtccgaaggcctcgaaggccgatcccaaagccccgtggccctgctctttgatgcccttctacgcccagacacagactttgggggaaacatgaagtcggtcctcacctggaagcaccggaaggagcacgccatcccccacgtggttctgggccggaacctccccgggggagcctggcactccatcgaaggctccatggtgatcctgagccaaggccagtggatggggctcccggacctggaggtcaaggactggatgcagaagaagcgaagaggtcttcgcaacagccgggccactgccggggacatcgcccactactacagggactacgtggtcaagaagggtctggggcataactttgtgtccggtgctgtagtcacagccgtggagtgggggacccccgatcccagcagctgtggggcccaggactccagccccctcttccaggtgagcggcttcctgaccaggaaccaggcccagcagcccttctcgctgtgggcccgcaacgtggtcctcgccacaggcacgttcgacagcccggcccggctgggcatccccggggaggccctgcccttcatccaccatgagctgtctgccctggaggccgccacaagggtgggtgcggtgaccccggcctcagaccctgtcctcatcattggcgcggggctgtcagcggccgacgccgtcctctacgcccgccactacaacatcccggtgatccatgccttccgccgggccgtggacgaccctggcctggtgttcaaccagctgcccaagatgctgtaccccgagtaccacaaggtgcaccagatgatgcgggagcagtccatcctgtcgcccagcccctatgagggttaccgcagcctccccaggcaccagctgctgtgcttcaaggaagactgccaggccgtgttccaggacctcgagggtgtcgagaaggtgtttggggtctccctggtgctggtcctcatcggctcccaccccgacctctccttcctgcctggggcagggctgactttgcagtggatcctgaccagccgctgagcgccaagaggaaccccattgacgtggaccccttcacctaccagagcacccgccagagggcctgtacgccatggggccgctggcggggacaacttcgtgaggtttgtgcaggggggcgccttggctgtggccagctccctgctaagaaggagaccaggaagccaccctaacactcggccagacccgctggctcccaggccctgagaggacagagatgaccacatccctgctggatgcaggacccgtccaaagatgccccggggaggggtgtcagcccacgttgctggcctttggggtcaagaggagtagggatcccaggctgccctggacttagaccagtgtgtgaggttggacttagaccagtgtgtgaggtggtaacagcggccgcagcagggggttggcctagacctgggatttgtggggaaagctgctggtgtgaccagctgagcacccagccaggagacctgcagccctgcgccttccagaagcaggtcccaaataaagccagtgcccacctgaaaaaaaaaa

SEQ ID NO: 2 - protein, 317 aa, Human
MKSVLTWKHRKEHAIPHVVLGRNLPGGAWHSIEGSMVILSQGQWMGLPDLEVKDWMQKKRRGLRNSRATAGDIAHYYRDYVVKKGLGHNFVSGAVVTAVEWGTPDPSSCGAQDSSPLFQVSGFLTRNQAQQPFSLWARNVVLATGTFDSPARLGIPGEALPFIHHELSALEAATRVGAVTPASDPVLIIGAGLSAADAVLYARHYNIPVIHAFRRAVDDPGLVFNQLPKMLYPEYHKVHQMMREQSILSPSPYEGYRSLPRHQLLCFKEDCQAVFQDLEGVEKVFGVSLVLVLIGSHPDLSFLPGAGLTLQWILTSR

SEQ ID NO: 3 - DNA, 954 bp, Human
  CDS (1)...(954)
atgaagtcggtcctcacctggaagcaccggaaggagcacgccatcccccacgtggttctgggccggaacctccccgggggagcctggcactccatcgaaggctccatggtgatcctgagccaaggccagtggatggggctcccggacctggaggtcaaggactggatgcagaagaagcgaagaggtcttcgcaacagccgggccactgccggggacatcgcccactactacagggactacgtggtcaagaagggtctggggcataactttgtgtccggtgctgtagtcacagccgtggagtgggggacccccgatcccagcagctgtggggcccaggactccagccccctcttccaggtgagcggcttcctgaccaggaaccaggcccagcagcccttctcgctgtgggcccgcaacgtggtcctcgccacaggcacgttcgacagcccggcccggctgggcatccccggggaggccctgcccttcatccaccatgagctgtctgccctggaggccgccacaagggtgggtgcggtgaccccggcctcagaccctgtcctcatcattggcgcggggctgtcagcggccgacgccgtcctctacgcccgccactacaacatcccggtgatccatgccttccgccgggccgtggacgaccctggcctggtgttcaaccagctgcccaagatgctgtaccccgagtaccacaaggtgcaccagatgatgcgggagcagtccatcctgtcgcccagcccctatgagggttaccgcagcctccccaggcaccagctgctgtgcttcaaggaagactgccaggccgtgttccaggacctcgagggtgtcgagaaggtgtttggggtctccctggtgctggtcctcatcggctcccaccccgacctctccttcctgcctggggcagggctgactttgcagtggatcctgaccagccgctga

SEQ ID NO: 4 - protein, 317 aa, Human; identical to SEQ ID NO: 2

SEQ ID NO: 5 - DNA, 12235 bp, Human
  mRNA (5808)...(5830): Exon 1
  mRNA (7003)...(7194): Exon 2
  mRNA (7415)...(7506): Exon 3
  mRNA (11497)...(12235): Exon 4
  CDS (7105)...(7194)
  CDS (7415)...(7506)
  CDS (11497)...(12235)
agctgtgtccctgtccaggtgcctggtaccctcagccaagcagctgaagcggccacagtcccagagtaccctgggccatatgaccgccgctcctagcaggaaagaggggtgtggccagcccgggcttactgcccagctgttccaaggccactgggtcggcagccaggctgggtgccttctgggtttctgcttctcctgctcaggagggacagagcccttgggtgcccatgaacagcccccatccctggaggtcccagcgacttagcttctctccggaggctcaagagtgcggcgggttgggagggcggcccctgggaggctggggagctgcaggagaccctgggctccaggtgtgcggcactggggcgagggctcgtctgtgataaatggcaagtgccgcttgcgtggacagaccaccccctcacactttagcccccccagggcttcgtgtcactttcttggtctccctgtaatcagctgttttactggaggggagttggcagaactaagatttgtcacatggccgtggtcttggaccctcctagttgacctggggcaagacgcccactcctcctgaccagcttttcttcctctgcacaatggggataagaacacccacctcctggggtgtcttcaaaggcaaaggagtaaatggccactagaggtgccaaagaaacgcagacctacaggtgtcccagagtctcagagcagtttgcagctttaacaacctcagaagtacaaatgccgcacacttgaaagaaacgtcagtcgaaggtgcaactgttttcatttcttttatcctgtttgcctttgtgaaatttgaaatatcaatttttaatgttaacttctttccatcaacatctgccatcttcaattaaagggacttaaaaagataacattaaaaattgattcttccaactccgtaaaactaaaaagcacaaaaggaattgaaatgaaacttttgaactctcttttgtaagttggcagcatatttgtccgaggttatgagtgacagcgtcaagctgcttcggactttggggcgtcctgtgtcactctgaacgggaagtgacttctcaggtccagcccctgggtggtgacctcatgtatgtggcatccagaagcttctgttcaagtgcctgagaacagtcccaggagggagccctggacttggagccccaaagccctggtggtaaaccagtgtcctctgctgcctgcaaagggtgggaaggggcagccagttctgattcctcactggccagcaggggctcctgcaaatgcccttcttggggtggtgggaaaatctgaaagaacaaaggaggggcagggcatggcccagggtgggcgcactggggcagatggagcaggttggctcactcgtttggtgaaggggctggggtgcctgggggtgtcagcccacggggatcaagtatcagttccatcgcccaccggctctgtagcctcagggcacggcttcacctctcctagccttagtctcctcatctgcaaagtgggttgctaggaggatcaaaggaggtgatgcggggaagccctttaccaggaggtgccatgctccccagtggctgctgttcccggggaccatgatgaggactgagggggcccagagccattcgagggtaccaggagcaagccagagaaagcgctctgagacccgtagtcctgttctccaaggcctctgggtatacccagcacccagggtcaatgagaaatgatcccttcccagccagaacaccaggtcctcagctgtccctctgcctccccgcctgccactagcactgcgacagagaagtgggggaccctggaacggacactgcacgttccacccggcagaggggtctcagcaggacctctcacagcacttggggcacagagaaggctctggagcagggcggcctgggctctgatccagcccagcctgcaagagctggttcaaccaccttgcaaactataaagggcggtgagaaacgctctggccctcagagtacttacaacaacccgattaaataaggattcaatttgttaacgtgtattaagtgttgagatcatgcctggcacccagcccactccacagaagtatggaaatgtcagccacatgctgtctttgttgttcttgtaactactactactattataattggtggcagcgtccccctgaaatctccccctcaaggcaggcagcctggggtcttgtagggagctgagcatgaagaggccatgtggtccctggtgactgcagcatgtcaccttccttctctctggaaagggaggaggaggtcttcggtgctgggcacacgagcttgcctagacaggacacggaggctgtccctggcccccacgccagtggaggcaagaaaggctatgctcaagggagcagagagcaccgacacaggggtccctgacagaccccttccccgctaggcacatcctcaccctcggagcctccttctcctcacctgcaagacagaggtgacagggctcacctctcaaggctgctgagcgggctggacgctactgcttagatgcaacctccttgcaaactgtagagtgctctgagaaggtcagttgttgtcactgtgggggcgacacataggtcctctcagtggcccatctgtgtggcagtttctcatccctctcctcgggacctgcctggaggtggacgatggtgtttctgtcaccctgcacacatcagaggccacacactggtctcagctgtgtttggttggccttttaagcatcttccaaaactgtgcgttcattcattgcccacttcttcaactcagatcattcacatagaaatccagattcccactttctctggaagaattctggaggcaggtggccctgggcccccacccctggccactccgggtggtccaagccctgtgcccgcccacttgagtgtgacacctggatgggccctggatctcgagggacaggccgagtggtgtgggtggtggcactggcagggtcttggcaccagtgagccctgggtttcaaagccrgctgtgtgacactgagaagtttgtgcgacctctctgagccccgtttacacatctgtgaaatgggacattaatatggccagggctgcacagggtcagcgtgaggcggatatgagaaaaagcatgtcaaatacccggtacacagtaagtgccaaatgctcatcatccctgtgcccacaagggtgttgccttgtggtcctcaggatggcagcatgtggccctcgggtgaggctggtgtccgctgctagctggtgtggggaagaaggtaggtctgatggtcctctgggaaccggcagctctaggaccagcacttgggtccctttgagcgatttgggcctgcggccagcagagggcgcggctgccccagagatggcaggttcaccagcacgtccccaaacctggccactgcttccatttccccaagtccatcccagctcctggggcaggccacagcgctggcccattccctgtggcagattcactccaggcaggtgcttcccgtctgtgatcctactgtgtccctcaaccaccctaggaggtagggactcattgcttccattttacagatgaggaaactgagacccagagaggcccgggaatgggataggaagcaacgtgccagggctggatgacgagtccaccaggccacactgggactcgaggactgtggcccacagaagggtctggcagggctggttcctgggttgggcaggaatggcctctcgggaagacagataatgagtttcggggactgttactgccccagtgagatgggagagtagagtgaggctcggagggacagtggctgcctggggcaaccaggcccgaggaggggagggcacattctgcatgactggggaggggtggggcaaacagcctgtttggagcagaggctgtgtgagagcaagaagggagtcccgtcagatatgtctcatggggaccccggaccagaccctgagggccacaggaggccaccctagctgggaggtgaagtggctgtgtggacacccaggcctcacccgaatggactcttccttcccctctcccccactccaggtccgctgccagccccaagccccccaccagccatgagctcctccagaaaggaccacctcggcgccagcagctcagagcccctcccggtcatcattgtgggtgagtgtcaggccccagccagggaggggctccgctgagccttccgggtaccccccaggtctgagggcaggagctgggcaagtccaggcctggtgtcttttttcgtttttggggtttattttttatttatttatttatttatttatttatttatttgagacaaagtctcactctgtcgcccaggctggagtgcagtagtgcaatcttggttcactgcaacctctccctaccgggctcaagcgattctcctgcttcagcctcccaagtagctgggattacaggtgcatgccaccatgcccagctaacttttgtatttttagtagagatggggtttcgccatgtcggccaggctggtctcgaactcctgacctcaagtgatctgcctgcctcagcctcccaaagtgctgggattataggtgtgaaccaccatgcctggcccaggcctggcctctttacagtggtctttaaattaagccattgagcgtttactatgtggccagtgctaggcttagcagtttataagctatcattccctttcacacttccgagaacccaggggtctagaacacacccatcagctggaggcgacccggcgtggcgcagggaggcctggtcctgagcccacactgatcccatgactaccctgtgcccagcactgtgctaggtgcccacatgagaggacacacagtagcggcttatgattcctgtcctctgtctgcacaaatggggacagatgaccctccagtatgcaggcgtccaattaagccacgtgagatggtcatttctgtaaggcaaaagcagatgggtgcctgtgatttcatatggttcttcctggtatcctgcatatgagtaatattccgaatcagacgagataagaagaggggaaaggacagataaaaagagaagcgctatcacagggctgtgcctggtgaatcgcccaggaagcgccacagatggcagcgcagttcttcagacgtgcaagcagcagacccaacaggcggctcagcacccctgagcgcccacaagcacccaccactcaagttcgcccccaggccacgtagcaatctgtgctacattcactcatccatctgacctagttcttagcaatctcagaatctcagtcgcgtcgtcagatgttccctctgtctgggttttaatcccggctcgtctaccttccggccgtgtgaccacaggcaagtaacttaacctctctgtgcccatttcctcaagtataaaaggaggaaaagagtggcacttacctcgctaaagctgttgtgaggctgttgtgaaagctctcagtacaattcctggcacagagtaagctctcagcagttgttcattccttctttttaaaagtattattaggcacagtgtctggcacaggctaagggcttaataaatatttatttttatgaatgaaggataaatgaatgaatgcgccacatctagatcaaagccctccacagtagtgtcccccacctgaaaaggccaccccctttaacctccaccctctctccccaggtaacggcccctctggtatctgcctgtcctacctgctctccggctacacaccctacacgaagccagatgccatccacccacaccccctgctgcagaggaagctcaccgaggccccgggggtctccatcctggaccaggtgggtcagcctggggccagagctctgggtagtacatacccgcccttggaaaactaccgccgctttcccagggaaaaacaaaagtgtgtgtttgctggaaacaagactaaacaaagaattcgagagtccacaaagtcagccacggagatcgtgaaagagaaatcagagacccacaaatgccacacgatctagggctcactatgccctaggccggttgttctcaaggaggggatgatttttctcctcggggggcacctggcaatgtctggagacaatttgggttgtcatggcagggtgacggtactaccaaatctagcagggtggaggccagggatgctggcaaacatccagcactgcacaggacaacgagggtggtgagactgaaatatgtgtggtggagcttctcaaccttggccccactgacatttggatgactttgttctggctacaaagaatgatctggctcaaatgtcagtgcggccaaggttgagaaactccaccacagatatttcagtcccactacctcctgcccacaagggtttgcaaatcctccctccgtaccccttgtcttccactgtgctcctccacgacctccccttgtcgacctccctcgtcaggcagtgtacatcctcctgttcccttcccgcccctgagttggagatgaggaaataggtcttgaccatttgatctagatcagcatttttcaaagtgctctctatagaatactagttcctcagtataatatagagagaaaaatgtccccactccagtaaaacagggacacactgggtttggcaaaattaaacaggtaaatgatggctgcaggacttgtcagagcctttaatatgcccgtctgtgttatctgctgttaagaggcagcacctcccaagctgatctgaccacactgatattagggtagggatgtaggaccaggcaatcgccccagtctcacccaagcttgcgcatttggtctgtactctggggcccttggcctcctagaaagacctcaaaacgagagcttcaaactacccccagggtgcgcatctctctgagaccctccagtgccagggaaatggcccggccccagtccccacgttcctgcccagcctcaggctctgggaagacgaccccgccctcctccagcagcccctctgacctatgcccccctccaggacctggactacctgtccgaaggcctcgaaggccgatcccaaagccccgtggccctgctctttgatgcccttctacgcccagacacagactttgggggaaacatgaagtcggtcctcacctggaagcaccggaaggagcacgccatcccccacgtggttctgggccggaacctccccgggggagcctggcacgtgagtggggcagcgagggcatggcttgtggggggctctctccctcgttctccccacttggggctgggactctggcatctccctttcctcattctccaccccgcaaccctgcctctccctcctccctctacccagccccagcaaaggcctcccatgccctctagccccaaatgggttcaggcgagcagaggcctggaccaaagtgttcctttccctgcagtccatcgaaggctccatggtgatcctgagccaaggccagtggatggggctcccggacctggaggtcaaggactggatgcagaagaagcgaaggtgaggccgccccggaacgccttgggggacacggaaggttgggcctgtgaacccagaaaatctgagacaggtctcagttaatttagaaagtttatttttccaaggttgaggacgcgcccgtgacagcctcaggaagtcctaatgacatgtgcccaaggtgatccggcacagcttggttttatacattttagggagacatgagacatcaatcaatatttgtaagaagtacattggttcggtctggaaagctgggccagcttgaagcaaaggcaggaagtggggagggggcttccaggtcgcaggtaggtgagagataaacagttgcattcttttgaggttctgatgagcctctccaaatgaggcgatcagatacgcatctatctcagggagcagaggggtgacttggaatggaatgggaggcagggtggccctcagcagttctcagcttgacttttccctttagcttagtgatttgggggatttcacaggccccccacagtgagcccgtgagccagaggtacaggggcttcagagtgcatctggtctgtcttcctcttcttccagagggggaaactgaggctcagatagggaaaggacttgtccaccccacatgagtgggggtgagcagcagagctgggctcacccgaggaccccctgtcccgtgcaagcagcagaccgccctgtcacatggcccccaactgggcctccccatagtgccccagcctctaaggagggggtaacacctggcccctgagccccacgccctgccccgtgcctctggcacaggtcccccagtccttgtggtctcccttcagccctttcctctattctgagcctggcctagaacaatctttctgggtcagctaatagggtgactttcttttttcttttcttttttttttttttaatgcttttgctacaggtgagtttcatccttaaccatgagcccccaagttgggtaaattaactcacctaaggtcatgcaggttattggaaatgcagaaggaaaacagctcaccccaaacccacacttctccacgccctttcactcctggggtattttttccccatctggcttcctgccccatcatggccaccctagagaaaaggtttgtgaaccctgaatatttgagatgggtctcagttaatttaggaagttttgtttgtttatttgttgtttgtttttaatgaagtctccctctgtcgcccaggctggagtgcagtggcgcgatctcagctcactgcaagctccgcctcccaggttcaagccattctcctgccccagcctcccgagtagctgggactacaggtgcccaccaccacacccggctaattattttttgtatttttagtagagacggggtttcaccgtgttagccaggatggtctcgatctcctgatctcgtgatccgcccacctcggcctcccaaagtgctgggattacaggcgtgagccaccacgccaggctaatttaggaagtttaattgccaaggttgaagacgcacaaccatgacacagcctcaggaggtcctgataacatgtacacatggtggtcggggcacagcctggctgtatacgttagggagacttgagacatcaatcactatatgtaagatggacattggttccgtccagaggcgggacaactccagcagggaggggtgtttccaggtctcagacaggtgagagacaaacggttgcattctttcgagtttcttataagcctttccaaaggaggcaatcaggtgtgcatttatctcagtgggcagagggaagactttgagtaggacgggaggcagctttgccctaggcagttccctgcttgacttttcctttaacttagtgatttgggggccccaagattgatttgcctctcacagtttcaatcactgaataaaggagttctagtccctgctcctcctggggtgcatccactcaatgagaagctgccccagcgcagatacctgggtgcagtctgcctctcgggaagccctgcttcctccgcccccagggctgtgaccaggcctgtgtccccaacggcaggcactccaggcgtccccctccaggcctcactatctctttgatgcgaagactcttctgcatgtttggcaggaagacttccaggtttgtgtagctacatgttagtaggcagccagaaacttacatttcgcttgtaagcacagccttttataggctcattttgcattttcacattgtatcccaagcgtttcatgtggtgaagtcacagcgttcaaaaccatccttttacatggcctcacaatattcccaaaggcgagtgagcatccccagtccagcaaatcttcaccgaatgcttattatgtgccaggccttgggcttagacgtcagagcacaaaaccaccgcagacacaccccctgcctcatggcactgccaaggttacccagcccctgccagccctggagcattcattctttccattgtgtcattattataaataactctgcacagaacatgctcttgggaccgttctgtgtcacaatctttacaaaaactgagaaaagaatgactgcccacagagataaattccagggtcgcagacgggctgcgctgggttgactggcacggccttgggggtccattgtcagctcctgctacatgtagccggacggcaggtggcacaggattctcccgtatgggagttcatcttgaaccatgagcccacaagctgggtagattaactcatctaaggtcacacagtgagatacaggaaggtgctagaatgatgcaaagcctgtcattcaaccgccacaagaatgggcctgcagtcactgcccctggaaagagtgacaatccaaacaacaacaacaataatagaatacctcctgctgctgcgtgggctgggaacgatggtgagtgcgtcacatgggagtctttaaatcaactttaaatcaactttttgactacatataggctgggtgcggtggcgcaagcctgtaatcccagcactttgggaggccgaggctggtggatcacttgaggtcaagagttcgaaaccatcctggccaacatggcaaaaccccatctctaataaaaatacaaaaattagccaggtatggcgctgcatgcctgtagttccagctgctcaggaggccgaggtatgagaatcacctgagcccgggaggcaggggctgcagcgagctgagatcacaccacggcactccagcctgggcagcagagtgagactctgtctcaaaaaattaaaataaaataaaataaaataaaataaaataataaattggctacatatagaaaagtacgtaactcctgtgagtacaggttgatgaaatttctcaaggtaagcacagagaatgaagaagcaggccccctcccagaagccccccatgccctttctgatcaggatcccattccccatccccggaggtaacccctctcttgacatccagctccatagatgagttctgcctgtttctgaactctgtaaaaatggaatcataccatatttaatcatttgaggtgacaatcatggtggcctatccttacatggcacttacttccagtcaggttccatcctaagcactttgtgtttgtcagctcaccaaatcctccccacgacctcacgaaataggtcctgccaccaacatcttcctcattttgcaaactggggaaacagacacagctccctgatggcagagtcaggattcgagcccaggcagcggggctctggaggctgccctctgaaccccaccctacaccaccctagagcatcctacaccaccctacaccacctctcctgagggaggcacccttgttagctccattttacagaagaggagactgaggcttagagaggttgaattacctggcctagtcctacagctacagcctagtccagctccagggcctgacactgccctggacatctcccgtcccacccagccccccaacccctaacagtgtctgactctggcgtcctgcatcctccccaacagaggtcttcgcaacagccgggccactgccggggacatcgcccactactacagggactacgtggtcaagaagggtctggggcataactttgtgtccggtgctgtagtcacagccgtggagtgggggacccccgatcccagcagctgtggggcccaggactccagccccctcttccaggtgagcggcttcctgaccaggaaccaggcccagcagcccttctcgctgtgggcccgcaacgtggtcctcgccacaggcacgttcgacagcccggcccggctgggcatccccggggaggccctgcccttcatccaccatgagctgtctgccctggaggccgccacaagggtgggtgcggtgaccccggcctcagaccctgtcctcatcattggcgcggggctgtcagcggccgacgcggtcctctacgcccgccactacaacatcccggtgatccatgccttccgccgggccgtggacgaccctggcctggtgttcaaccagctgcccaagatgctgtaccccgagtaccacaaggtgcaccagatgatgcgggagcagtccatcctgtcgcccagcccctatgagggttaccgcagcctccccaggcaccagctgctgtgcttcaaggaagactgccaggccgtgttccaggacctcgagggtgtcgagaaggtgtttggggtctccctggtgctggtcctcatcggctcccaccccgacctctccttcctgcctggggcaggg

SEQ ID NO: 6 - protein, 307 aa, Human
MKSVLTWKHRKEHAIPHVVLGRNLPGGAWHSIEGSMVILSQGQWMGLPDLEVKDWMQKKRRGLRNSRATAGDIAHYYRDYVVKKGLGHNFVSGAVVTAVEWGTPDPSSCGAQDSSPLFQVSGFLTRNQAQQPFSLWARNVVLATGTFDSPARLGIPGEALPFIHHELSALEAATRVGAVTPASDPVLIIGAGLSAADAVLYARHYNIPVIHAFRRAVDDPGLVFNQLPKMLYPEYHKVHQMMREQSILSPSPYEGYRSLPRHQLLCFKEDCQAVFQDLEGVEKVFGVSLVLVLIGSHPDLSFLPGAG

SEQ ID NO: 7 - DNA, 1723 bp, Mouse
  CDS (3)...(1373)
  polyA_signal (1703)...(1708)
  polyA_site (1723)...(1723)
tcggcaatggcccctccggtatctgcctgtcctacctgctctctggacacatcccctacgtgaaaccaggagccgtccacccacaccccctgctgcagagaaagctggccgaggctacaggggtctccatcctggaccaggacctagagtacctttccgaaggcctcgaaggccgaagccagagtcctgtggccctgctctttgatgccctcctgcgtcctgacacagactttggaggcagcatagactctgtgctctcctggaagcggcagaaggaccgcgctgttccccaccttgtcctgggacggaacctccccgggggcgcctggcattccattgaaggctccatggtgaccttgagccaaggccagtggatgagtctcccagacctgcaggtcaaagactggatgcggaagaaatgcagaggtctccgcaacagcagagccacagccggtgacatcgcccactactaccgggactacgtgatcaagaaaggcctaagtcacaactttgtgtctggtgccgtggtcactgccgtggagtgggctaagtccgagcatggcagtcccgaggtccaggcatccagtcccctcttccaagtgaatggctacttgactaccaaggaccacggccaccagcccttctcactgcgggcccgtaatgtggtcctggccacaggcacctttgacagcccagccatgctaggcatcccaggagagaccctgccctttgtccaccacgacctgtcagccctggaggcagcccttcgggcaggcactgtgaacccaacctcagacccggtgctgattgtgggagcagggctatctgcagccgatgctgtcctctttgcccgtcactacaacatccaggtgatccatgctttccgccggtccgtgcatgaccccggcctggtattcaaccagctgcccaagatgctataccctgagtaccacaaagtgcagcagatgatgcgcgatcagtccatcttgtctcctagcccctatgagggctaccgcagcctccctgagcaccagccactgctcttcaaagaggaccaccaagcagtgttccaggacccacaggggggccagcagctctttggggtctccatggtgctggtcctcattggctcccaccccgacctctcctacctccccagggcaggtgctgacttggtcatagacccagatcagccactgagtcccaagaggaaccccattgacgtggaccccttcacccatgagagcactcaccaggagggcctgtatgccctggggccgttggctggggacaactttgtgagatttgtccagggcggggccctggctgctgccagctccctgctgaagaaggagaccaggaagccaccttaacattagcctggcaccctgacacccaggccctgaagaggaagagggcttatcacagcccagcgggacgtgagcccatctaaagatttcccatttgggaaccagtctccccagcagaaggggaagacttgagccatgtggatggactatctgccccagaggcatggggaacacaggctgccctaccctcaggcgagatgaggccccaggtgggagcagtagccatagaccagggggcccccttgaaactgtcagtgtgtgctggagtcacagggccagctgagtgtccaacttggagggccccaggcccaacttttcaccatcagatctgtaataaaaccagctgtgcctcc

SEQ ID NO: 8 - protein, 456 aa, Mouse
GNGPSGICLSYLLSGHIPYVKPGAVHPHPLLQRKLAEATGVSILDQDLEYLSEGLEGRSQSPVALLFDALLRPDTDFGGSIDSVLSWKRQKDRAVPHLVLGRNLPGGAWHSIEGSMVTLSQGQWMSLPDLQVKDWMRKKCRGLRNSRATAGDIAHYYRDYVIKKGLSHNFVSGAVVTAVEWAKSEHGSPEVQASSPLFQVNGYLTTKDHGHQPFSLRARNVVLATGTFDSPAMLGIPGETLPFVHHDLSALEAALRAGTVNPTSDPVLIVGAGLSAADAVLFARHYNIQVIHAFRRSVHDPGLVFNQLPKMLYPEYHKVQQMMRDQSILSPSPYEGYRSLPEHQPLLFKEDHQAVFQDPQGGQQLFGVSMVLVLIGSHPDLSYLPRAGADLVIDPDQPLSPKRNPIDVDPFTHESTHQEGLYALGPLAGDNFVRFVQGGALAAASSLLKKETRKPP

(19) United States
(12) Patent Application Publication
(10) Pub. No.: US 2003/0166519 A1
(43) Pub. Date: Sep. 4, 2003

(54) Novel polynucleotides and polypeptides and uses therefor

(75) Inventor: The Hung Huynh, Singapore (SG)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: National Cancer Centre of Singapore Pte Ltd., Singapore (SG)

(21) Appl. No.: 09/815,453

(22) Filed: Mar. 22, 2001

(30) Foreign Application Priority Data
Aug. 15, 2000 (AU) PQ 9470
May 25, 2000 (GB) 0012820.7
May 24, 2000 (AU) PQ 7732

(51) Int. Cl. A61K 48/00; C12P 21/02; C12N 5/06; C12Q 1/68; G01N 33/574; C07H 21/04; C12N 9/00

(52) U.S. Cl. 514/12; 514/44; 435/6; 435/69.1; 435/7.23; 435/325; 435/183; 536/23.2

(57) ABSTRACT

The present invention relates generally to agents that modulate growth regulation and cell differentiation. More particularly, the present invention relates to novel OKL38 polypeptides that modulate growth regulation and differentiation of mammary epithelial cells and to polynucleotides encoding these novel polypeptides. The invention also relates to biologically active fragments of the novel OKL38 polypeptides as well as to variants and derivatives of these polypeptides. The invention further relates to the use of the polypeptides and polynucleotides of the invention in compositions for treating and/or preventing conditions that are associated with aberrant OKL38 expression or that are ameliorable by OKL38 expression. The invention also extends to modulatory agents that modulate OKL38 expression and to the use of these agents for prophylactic and/or therapeutic purposes. Further, the invention relates to antigen-binding molecules that are immuno-interactive with the polypeptides of the invention and to the use of these antigen-binding molecules for diagnostic purposes.

### FIELD OF THE INVENTION

THIS INVENTION relates generally to agents that modulate growth regulation and cell differentiation. More particularly, the present invention relates to novel OKL38 polypeptides that modulate growth regulation and differentiation of mammary epithelial cells and to polynucleotides encoding these novel polypeptides. The invention also relates to biologically active fragments of the novel OKL38 polypeptides as well as to variants and derivatives of these polypeptides. The invention further relates to the use of the polypeptides and polynucleotides of the invention in compositions for treating and/or preventing conditions that are associated with aberrant OKL38 expression or that are ameliorable by OKL38 expression. The invention also extends to modulatory agents that modulate OKL38 expression and to the use of these agents for prophylactic and/or therapeutic purposes. Further, the invention relates to antigen-binding molecules that are immuno-interactive with the polypeptides of the invention and to the use of these antigen-binding molecules for diagnostic purposes.

Bibliographic details of the publications numerically referred to in this specification are collected at the end of the description.

### BACKGROUND OF THE INVENTION

Breast cancer is the most common cancer and the second most prevalent cause of cancer death in women. Globally, the incidence of breast cancer appears to be increasing and an annual world-wide incidence of over one million is predicted by the turn of this century (1).

Epidemiological studies have demonstrated that for women early age at menarche, late age at first pregnancy and late age at menopause tend to increase the risk for breast cancer (2, 3). The lifetime risk of breast cancer is 2 to 5 times higher in women who have first pregnancy after age 30 than in women whose first pregnancy is at an age younger then 20 (2, 3). It has been hypothesised that first pregnancy at a young age may differentiate breast cells early in life, after which they would become less susceptible to carcinogens (4-6). This hypothesis was supported by the observation that in animal models, mammary tumorigenesis is facilitated when the administration of carcinogen precedes pregnancy, however it decreases when the carcinogen exposure occurs during pregnancy (7). Normal and prolonged lactation in mice and in rats is also recognised to result in a decrease in the incidence of spontaneous or carcinogen-induced mammary tumours and an increase in tumour age when compared with forced breeding without lactation. In accordance, mammary DNA synthesis is at a very low level during lactation in mice and rats. These observations in experimental animals show protection of pregnancy and lactation against mammary tumorigenesis.

Both animals and humans are constantly exposed to carcinogenic agents during their lifetime. It is conceivable, therefore, that the longer the total period of low mammary DNA synthesis, i.e., proliferative mitotic rest, owing to pregnancy, lactation, etc., the smaller is the risk of mammary malignancy. In humans, breast parenchymal growth and DNA synthesis are pronounced only during the first trimester of pregnancy. The latter half of pregnancy is the period of proliferative and mitotic rest and breast parenchyma shows only minor DNA synthesis. The protective effects of having children early in life may accrue by causing breast cells to become more differentiated. It is impractical to suggest early pregnancy as a breast cancer prevention strategy, but an investigation of the physiological basis of this protection may lead to novel risk reduction strategies.

Peptide growth factors and inhibitors play key roles in regulating the proliferation of normal breast epithelium (8). The importance of peptide growth factors in the pathogenesis and behaviour of breast neoplasms is evident in the large amount of literature that has accumulated in the past decade concerning the roles of EGF, IGFs, TGF-α, and FGF (8, 9). To date, the best-characterised inhibitor is TGF-β (10). A negative regulatory function for insulin-like binding protein 3 (11-14), and for mammary derived growth inhibitor (15) have been reported. Abnormal expression of growth factors and growth inhibitors has been implicated in tumorigenesis (15-17). These observations suggest that interruption of growth factor action (or production) or enhancement of growth inhibitor production by breast cancer cells would represent new strategies for arrest of tumour growth.

### SUMMARY OF THE INVENTION

The present invention arises in part from the unexpected discovery of a novel gene that modulates differentiation and proliferation of epithelial cells, particularly of mammary epithelial cells. The expression of this novel gene, designated OKL38, is induced by pregnancy and lactation, by hormone associated with pregnancy such as hCG, and by drugs that inhibit breast cancer cell growth such as taxol, doxorubicin, tamoxifen and ICI 182780. The present inventor has also shown that OKL38 expression is low in breast cancer cell lines and barely detectable in DMBA-induced breast tumours and that transfection of human MCF-7 breast cancer cells with OKL38 cDNA leads to reduction in proliferation and tumour formation in nude mice. It has also been shown that OKL38 is expressed in all tissues examined with the highest levels detected in ovary, kidney, liver, testis, bladder and lung.

The above discoveries have been reduced to practice in new isolated molecules for use in modulating at least one activity selected from the group consisting of cell proliferation, cell differentiation and tumorigenesis, and in compositions for treating and/or preventing conditions that are associated with aberrant OKL38 expression or that are ameliorable by OKL38 expression as described hereinafter.

Accordingly, in one aspect of the invention, there is provided an isolated polypeptide, or a biologically active fragment thereof, or a variant or derivative of these, said polypeptide comprising the amino acid sequence set forth in SEQ ID NO: 2.

Preferably said fragment consists essentially of at least 8 contiguous amino acids of the sequence set forth in SEQ ID NO: 2. In one embodiment of this type, the biologically active fragment is selected from residues 1-8, 9-16, 17-24, 25-32, 33-40, 41-48, 49-56, 57-64, 65-72, 73-80, 81-88, 89-96, 97-104, 105-112, 113-120, 121-128, 129-136, 137-144, 145-152, 153-160, 161-168, 169-176, 177-184, 185-192, 193-200, 201-208, 209-216, 217-224, 225-232, 223-240, 241-248, 249-256, 257-264, 265-272, 273-280, 281-288, 289-296, 297-304, 305-312 and 310-317 of SEQ ID NO: 2.

Suitably, the variant has at least 80%, preferably at least 85%, more preferably at least 90%, more preferably at least 95% and still more preferably at least 98% sequence identity to the sequence set forth in SEQ ID NO: 2. In a preferred embodiment, the variant is distinguished from the sequence set forth in SEQ ID NO: 2 by the substitution of at least one amino acid residue. In an especially preferred embodiment of this type, said substitution is a conservative substitution.

In another aspect, the invention provides an isolated polynucleotide encoding a polypeptide, fragment, variant or derivative as broadly described above. In a preferred embodiment, the polynucleotide comprises the sequence set forth in any one of SEQ ID NO: 1, 3 and 5, or a biologically active fragment thereof, or a polynucleotide variant of these.

Preferably said fragment comprises at least 24 contiguous nucleotides of the sequence set forth in any one of SEQ ID NO: 1, 3 and 5.

The variant may be obtained from any suitable animal. Preferably, the variant is obtained from a mammal.

The invention, in another aspect, encompasses a composition for promoting, augmenting or otherwise enhancing cell differentiation, comprising a member selected from the group consisting of a polypeptide comprising the amino acid sequence set forth in SEQ ID NO: 2, a fragment of said polypeptide, a variant of said polypeptide, a variant of said polypeptide fragment, a derivative of said polypeptide, a derivative of said polypeptide fragment, a polynucleotide encoding said polypeptide, a fragment of said polynucleotide, a variant of said polynucleotide and a variant of said polynucleotide fragment.

In yet another aspect, the invention envisions a composition for delaying, repressing or otherwise inhibiting cell proliferation, comprising a member selected from the group consisting of a polypeptide comprising the amino acid sequence set forth in SEQ ID NO: 2, a fragment of said polypeptide, a variant of said polypeptide, a variant of said polypeptide fragment, a derivative of said polypeptide, a derivative of said polypeptide fragment, a polynucleotide encoding said polypeptide, a fragment of said polynucleotide, a variant of said polynucleotide and a variant of said polynucleotide fragment.

In a further another aspect, the invention features a composition for delaying, repressing or otherwise tumorigenesis, comprising a member selected from the group consisting of a polypeptide comprising the amino acid sequence set forth in SEQ ID NO: 2, a fragment of said polypeptide, a variant of said polypeptide, a variant of said polypeptide fragment, a derivative of said polypeptide, a derivative of said polypeptide fragment, a polynucleotide encoding said polypeptide, a fragment of said polynucleotide, a variant of said polynucleotide and a variant of said polynucleotide fragment.

In another aspect, the invention contemplates a vector comprising a polynucleotide as broadly described above.

In yet another aspect, the invention features an expression vector comprising a polynucleotide as broadly described above wherein the polynucleotide is operably linked to a regulatory polynucleotide.

In a further aspect, the invention provides a host cell containing a vector or expression vector as broadly described above.

The invention also contemplates a method of producing a recombinant polypeptide, fragment, variant or derivative as broadly described above, comprising:

culturing a host cell containing an expression vector as broadly described above such that said recombinant polypeptide, fragment, variant or derivative is expressed from said polynucleotide; and

isolating the said recombinant polypeptide, fragment, variant or derivative.

In a further aspect, the invention provides a method of producing a biologically active fragment as broadly described above, comprising:

introducing a fragment of the polypeptide or a polynucleotide from which the fragment can be translated into a cell; and

detecting modulation of an activity selected from the group consisting of cell differentiation, cell proliferation and tumorigenesis, which indicates that said fragment is a biologically active fragment.

Preferably, said cell differentiation is epithelial cell differentiation, more preferably mammary epithelial cell differentiation and even more preferably mammary secretory epithelial cell differentiation. In this instance, the method preferably comprises detecting promotion of cell differentiation.

Suitably, said cell proliferation is epithelial cell proliferation, preferably mammary epithelial cell proliferation, more preferably mammary secretory epithelial cell proliferation. In this instance, the method preferably comprises detecting inhibition of cell proliferation.

In yet a further aspect, the invention provides a method of producing a polypeptide variant of a parent polypeptide comprising the sequence set forth in SEQ ID NO: 2, or biologically active fragment thereof, comprising:

providing a modified polypeptide whose sequence is distinguished from the parent polypeptide by substitution, deletion or addition of at least one amino acid;

introducing said modified polypeptide or a polynucleotide from which the modified polypeptide can be translated into a cell; and

detecting modulation of an activity selected from the group consisting of cell differentiation, cell proliferation and tumorigenesis, which indicates that said modified polypeptide is a polypeptide variant.

In a further aspect of the invention, there is provided a method for modulating at least one activity selected from the group consisting of cell differentiation, cell proliferation and tumorigenesis, said method comprising introducing into said cell a polypeptide, fragment, variant or derivative as broadly described above or a polynucleotide from which said polypeptide, fragment, variant or derivative can be translated.

The present inventor has determined that undesirable downregulation or inactivation of OKL38 is associated with reduction of cell differentiation and promotion of cell proliferation and tumorigenesis. It has also been determined that enhanced expression of OKL38 cDNA leads to enhancement of cell differentiation and to reduction of cell proliferation and tumorigenesis. Accordingly, the isolated polypeptides and polynucleotides as broadly described above can be used to provide both drug targets and regulators to promote or inhibit cell proliferation, cell differentiation and tumorigenesis and to provide diagnostic markers for cell proliferation and cell differentiation during normal or disease stages, e.g. using detectable polypeptides and polynucleotides as broadly described above, or using detectable agents which interact specifically with those polypeptides or polynucleotides.

Thus, in another aspect, the invention extends to a method of screening for an agent which modulates at least one activity selected from the group consisting of cell differentiation, cell proliferation and tumorigenesis, said method comprising:

contacting a preparation comprising a member selected from the group consisting of the polypeptide, fragment, variant and derivative as broadly described above and a genetic sequence encoding said polypeptide, fragment, variant or derivative, with a test agent; and

detecting a change in the level and/or functional activity of said member or an expression product of said genetic sequence.

In another aspect, the invention resides in the use of a polypeptide, fragment, variant or derivative according to the present invention to produce an antigen-binding molecule that is immuno-interactive with said polypeptide, fragment, variant or derivative.

In yet another aspect, the invention provides antigen-binding molecules so produced.

In another aspect, the invention envisions a method for detecting a specific polypeptide or polynucleotide sequence, comprising detecting a sequence of:

SEQ ID NO: 2, or a fragment thereof at least 8 amino acids residues in length; or

SEQ ID NO: 1, 3 or 5, or a fragment thereof at least 24 nucleotides in length;

In yet another aspect, there is provided a method for detecting a polypeptide, fragment, variant or derivative as broadly described above, comprising:

detecting expression in a cell of a polynucleotide encoding said polypeptide, fragment, variant or derivative.

According to another aspect of the invention, there is provided a method of detecting in a biological sample a polypeptide, fragment, variant or derivative as broadly described above, comprising:

contacting the sample with an antigen-binding molecule as broadly described above; and

detecting the presence of a complex comprising said antigen-binding molecule and said polypeptide, fragment, variant or derivative in said contacted sample.

In yet another aspect, the invention encompasses a method for diagnosis of a cancer or tumour in a patient, comprising:

providing a biological sample from said patient; and

detecting relative to a normal reference value, a reduction in the level and/or functional activity of a member selected from the group consisting of a polypeptide comprising the sequence set forth in SEQ ID NO: 2 or variant thereof, and a polynucleotide comprising the sequence set forth in SEQ ID NO: 1 or 3, or variant thereof.

In a further aspect, the invention envisions a method for diagnosis of a cancer or tumour in a patient, comprising:

providing a biological sample from said patient; and

detecting an aberrant OKL38 polynucleotide or OKL38 polypeptide.

In yet a further aspect, the invention features a method for diagnosis of a cancer or tumour in a patient, comprising:

providing a biological sample from said patient; and

detecting a loss of one or more copies of the OKL38 gene.

In yet another aspect, the invention encompasses a method for diagnosis of a cancer or tumour in a patient, comprising:

contacting a biological sample obtained from said patient with an antigen-binding molecule as broadly described above,

measuring the concentration of a complex comprising said antigen-binding molecule and a polypeptide comprising the sequence set forth in SEQ ID NO: 2, or variant thereof, in said contacted sample; and

relating said measured complex concentration to the concentration of said polypeptide in said sample, wherein the presence of a reduced concentration relative to a normal reference concentration is indicative of said cancer or tumour.

Preferably, the cancer or tumour is associated with an organ selected from the group consisting of breast, liver, kidney, testis, prostate, bladder and lung. In a preferred embodiment, the cancer or tumour is a cancer or tumour of the breast and liver. In an especially preferred embodiment, the cancer or tumour is a cancer or tumour of the breast.

In another aspect of the invention, there is provided a method for modulating at least one activity selected from the group consisting of cell differentiation, cell proliferation and tumorigenesis, said method comprising introducing into said cell an agent as broadly described above for a time and under conditions sufficient to modulate the level and/or functional activity of OKL38.

The agent preferably increases the level and/or functional activity of OKL38.

In another aspect, the invention provides a composition for treatment and/or prophylaxis of a cancer or tumour, comprising an agent selected from the group consisting of a polypeptide, fragment, variant or derivative as broadly described above, a polynucleotide from which said polypeptide, fragment, variant or derivative can be translated, and a modulatory agent that enhances the level and/or functional activity of OKL38, together with a pharmaceutically acceptable carrier.

According to another aspect of the invention, there is provided a method for treatment and/or prophylaxis of a cancer or tumour, said method comprising administering to a patient in need of such treatment a therapeutically effective amount of an agent selected from the group consisting of a polypeptide, fragment, variant or derivative as broadly described above, a polynucleotide from which said polypeptide, fragment, variant or derivative can be translated, and a modulatory agent that enhances the level and/or functional activity of OKL38 as broadly described above, and optionally a pharmaceutically acceptable carrier.

The invention also encompasses the use of the polypeptide, fragment, variant or derivative as well as the modulatory agents as broadly described above in the study, and modulation of cell differentiation, cell proliferation and tumorigenesis.

### BRIEF DESCRIPTION OF THE DRAWINGS

FIG. 1. Northern blot analysis of OKL38 gene expression in adult rat tissues. Two micrograms of poly “A” RNA derived from each tissue of 3 months old rat was subjected to Northern blot analysis. Blots were hybridised with GAPDH (A) and OKL38 (B) cDNAs. Densitometric scanning of the 1.6 kb band is shown in (C). Tissues are: He: heart; Ut: uterus; Lu: lung; Ov: ovary; Ce: cerebellum; Pr: prostate; Ki: kidney; Sp: spleen; Pi: pituitary; In: small intestine; Mu: red muscle; Li: liver; and Mg: mammary gland. Highest levels of OKL38 mRNA were detection in ovary, kidney and liver. High molecular OKL38 transcripts were also detected.

FIG. 2. Western blot analysis of OKL38 protein in adult rat tissues. Total proteins extracted from various tissues of 3 months old rat was analysed by Western blotting. Blots were incubated with rabbit polyclonal anti-OKL38 and mouse anti α-tubulin antibodies. Tissues are: Cer: cerebellum; Pi: pituitary; Ut: uterus; Ov: ovary; Mg: mammary gland; He: heart; Bla: bladder; Spl: spleen; Int: small intestine; Fa: fat; Li: liver; Lu: lung; Ki: kidney; Mu: red muscle. Highest levels of OKL38 protein were observed in heart, kidney, liver and cerebellum.

FIG. 3. Changes in OKL38 expression in the mammary gland during pregnancy and lactation. Two micrograms of poly “A” RNA derived from mammary gland on days 0 (Lane 1), 4 (Lane 2), 10 (Lane 3), 16 (Lane 4), 21 (Lane 5) of pregnancy and day 3 of lactation (Lane 6) were subjected to Northern blotting. Blots were hybridised with OKL38 (A) and GAPDH (B) cDNAs. Densitometric scanning of the 1.6 kb band is shown in (C). OKL38 transcripts increased following pregnancy and lactation. (D) Detection of OKL38 protein in the mammary gland during pregnancy and lactation.

FIG. 4. Effects of human chorionic gonadotropin on OKL38 gene expression. Female rats were treated with indicated concentrations of hCG for 3 weeks. Two micrograms of poly “A” RNA derived from mammary tissues were subjected to Northern blotting. Blots were hybridised with OKL38 (A) and GAPDH (B) cDNAs. Densitometric scanning of the 1.6 kb band is shown in (C). Note that OKL38 gene expression was significantly induced by hCG (p<0.01) which is known to induce mammary gland differentiation.

FIG. 5. Effects of estradiol, tamoxifen and ICI 182780 on OKL38 gene expression. Intact rats were treated with indicated concentrations of ICI 182780 (ICI), tamoxifen (TAM), and 17-β estradiol (E₂) for 3 weeks. Two micrograms of poly “A” RNA derived from mammary tissues were subjected to Northern blotting. Blots were hybridised with GAPDH (A) and OKL38 (B) cDNAs. Note that OKL38 gene expression was significantly induced by tamoxifen and ICI 182780 (P<0.05).

FIG. 6. Detection of OKL38 transcripts in human breast cancel cell lines. Two micrograms of poly “A” RNA derived from human breast cancel cell lines were subjected to Northern blotting. Blots were hybridised with OKL38 (A) and GAPDH (B) cDNAs. Positive control mammary gland RNA (Lane 1) and cell lines: MCF-7 (Lane 2); T47D (Lane 3); ZR75 (Lane 4); MDA-231 (Lane 5); Hs578T (Lane 6) and HBL-100 (Lane 7). Note that OKL38 gene expression was low in all breast cancer cell lines.

FIG. 7. Effects of taxol, doxorubicin, cisplatin on OKL38 gene expression. Human MCF-7 breast cancel cells were cultured with serum free media (Lane 1), 10⁻¹⁰ M taxol (Lane 2), 10⁻¹⁰ M cisplatin or 10⁻¹⁰ M doxorubicin for 2 days prior to extraction of mRNA for Northern blotting. Two micrograms of poly “A” RNA derived from treated cells were subjected to Northern blotting. Blots were hybridised with OKL38 (A) and GAPDH (B) cDNAs. Densitometric scanning of the 1.6 kb band is shown in (C). Significant upregulation on OKL38 gene expression by taxol and doxorubicin compounds (P<0.01), which are well known inhibitors of breast epithelial proliferation.

FIG. 8. OKL38 gene expression in the mammary gland during pregnancy and DMBA-induced breast tumours. Two micrograms of poly “A” RNA derived from normal mammary tissues of pregnant rats or DMBA-induced mammary tumours of pregnant rats were analysed by Northern blotting. Blots were hybridised with β-actin (A) and OKL38 (B) cDNAs. In pregnant rats bearing DMBA-induced tumours, the OKL38 expression seen in normal mammary gland is abundant relative to that seen in all mammary ductal neoplasms.

FIG. 9. Effects of stable transfection of breast cancer cell line with OKL38 cDNA. Northern blot hybridisation with a [³²P]dCTP-labelled OKL38 probe (B) of total RNA (50 μg/lane) extracted from parental MCF-7 cells (P), pcDNA3.1 vector transfected cell lines (Lines 1, 2 and 34) and OKL38 transfectants (SQ13 and SQ18). RNA loading amounts were compared by ethidium bromide staining of 18S and 28S rRNA (B). Immunodetection of 38 kDa OKL38 in stably transfected cell lines SQ13 and SQ 18 (C). Proliferative behaviour of the clones expressing OKL38 by determining cell number on plastic dishes after 8 days incubation (D). The number of cells was significantly less (p<0.05, Mann-Whitney U-test) in OKL38-expressing transfectants than in controls. Tumour formation of 38 kDa OKL38 in stably transfected cell lines SQ13 and SQ18 (E). The rate of tumour formation was significantly less (p<0.05, Mann-Whitney U-test) in OKL38-expressing transfectants than in controls.

FIG. 10. Schematic representation showing a map of chromosome 16 showing the location of OKL38.

FIG. 11. Diagrammatic representation showing the genomic organisation of the OKL38 gene, including the relative position of exons, the putative OKL38 promoter and their sizes.

FIG. 12. Northern blot analysis of OKL38 gene expression in adult human tissues. Two μg of poly “A” RNA derived from each human tissue was subjected to Northern blot analysis. Blots were hybridised with human OKL38 cDNAs. Tissues are: uterus, cervix, ovary, testis, prostate, lung, muscle, bladder, kidney, spleen, heart, brain, liver, pancreas, and placenta. The OKL38 mRNA band was observed in all tissues with the highest levels were seen in the kidney, liver, lung and testis.

FIG. 13. Gene pattern of OKL38 in normal and breast cancer tissues. DNA derived from normal human mammary tissues (lanes 1, 3, 5, 7, 9 and 11) and breast tumours (lanes 2, 4, 6, 8, 10 and 12). DNA was digested with PstI and analysed by Southern blot analysis with a human OKL38 cDNA probe. Samples 1 and 2, 3 and 4, 5 and 6, 7 and 8, 9 and 10, 11 and 12 were paired.

FIG. 14. Northern blot analysis of OKL38 gene expression in foetus, foetal livers of Lit+/−, Lit/Lit and Tgh GH mice and placenta. Blots were performed using total RNA from 7 day foetus Lit+/−, 10 day Lit/Lit foetus or 10 day foetal liver of Lit+/−, Lit/Lit and Tgh GH mice. Blots were hybridised with human OKL38 cDNA.

### BRIEF DESCRIPTION OF THE SEQUENCES: SUMMARY TABLE

TABLE A

| SEQUENCE ID | | | |
|---|---|---|---|
| NUMBER | SEQUENCE | LENGTH | |
| SEQ ID NO: 1 | Full-length human OKL38 cDNA | 1607 | bases |
| SEQ ID NO: 2 | Full-length human OKL38 poly- | 317 | residues |
| | peptide encoded by SEQ ID NO: 1 | | |
| SEQ ID NO: 3 | Coding sequence for full-length | 954 | bases |
| | human OKL38 polypeptide | | |
| SEQ ID NO: 4 | Polypeptide encoded by | 317 | residues |
| | SEQ ID NO: 3 | | |
| SEQ ID NO: 5 | Genomic sequence of OKL38 gene | 12235 | bases |
| SEQ ID NO: 6 | Polypeptide encoded by | 307 | residues |
| | SEQ ID NO: 5 | | |
| SEQ ID NO: 7 | Murine variant of human OKL38 | 1723 | bases |
| | cDNA | | |
| SEQ ID NO: 8 | Polypeptide encoded by | 456 | residues |
| | SEQ ID NO: 7 | | |

### DETAILED DESCRIPTION OF THE INVENTION

1. Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e. to at least one) of the grammatical object of the article. By way of example, “an element” means one element or more than one element.

The term “aberrant polynucleotide” refers to a polynucleotide resulting from a substitution, deletion and/or addition of one or more nucleotides in a “normal” reference polynucleotide.

The term “about” is used herein to refer to polypeptides that vary by as much as 30%, preferably by as much as 20%, and more preferably by as much as 10% to the length of a reference polypeptide.

By “agent” is meant a naturally occurring or synthetically produced molecule which interacts either directly or indirectly with a target member, the level and/or functional activity of which are to be modulated.

“Amplification product” refers to a nucleic acid product generated by nucleic acid amplification techniques.

By “antigen-binding molecule” is meant a molecule that has binding affinity for a target antigen. It will be understood that this term extends to immunoglobulins, immunoglobulin fragments and non-immunoglobulin derived protein frameworks that exhibit antigen-binding activity.

As used herein, the term “binds specifically” and the like refers to antigen-binding molecules that bind the polypeptide or polypeptide fragments of the invention but do not significantly bind to homologous prior art polypeptides.

By “biologically active fragment” is meant a fragment of a full-length parent polypeptide which fragment retains the activity of the parent polypeptide. A biologically active fragment will, therefore, inter alia modulate at least one activity selected from the group consisting of cell differentiation, cell proliferation and tumorigenesis, or elicit an immunogenic response to produce elements (e.g., antigen-binding molecules) that specifically bind to the parent polypeptide. As used herein, the term “biologically active fragment” includes deletion mutants and small peptides, for example of at least 8, preferably at least 10, more preferably at least 15, even more preferably at least 20 and even more preferably at least 30 contiguous amino acids, which comprise the above activities. Peptides of this type may be obtained through the application of standard recombinant nucleic acid techniques or synthesised using conventional liquid or solid phase synthesis techniques. For example, reference may be made to solution synthesis or solid phase synthesis as described, for example, in Chapter 9 entitled “Peptide Synthesis” by Atherton and Shephard which is included in a publication entitled “Synthetic Vaccines” edited by Nicholson and published by Blackwell Scientific Publications. Alternatively, peptides can be produced by digestion of a polypeptide of the invention with proteinases such as endoLys-C, endoArg-C, endoGlu-C and staphylococcus V8-protease. The digested fragments can be purified by, for example, high performance liquid chromatographic (HPLC) techniques.

The term “biological sample” as used herein refers to a sample that may be extracted, untreated, treated, diluted or concentrated from an animal. The biological sample may be selected from the group consisting of whole blood, serum, plasma, saliva, urine, sweat, ascitic fluid, peritoneal fluid, synovial fluid, amniotic fluid, cerebrospinal fluid, skin biopsy, and the like. Preferably, the biological sample is a tissue biopsy, preferably selected from breast, liver, kidney, testis, bladder and lung.

Throughout this specification, unless the context requires otherwise, the words “comprise”, “comprises” and “comprising” will be understood to imply the inclusion of a stated step or element or group of steps or elements but not the exclusion of any other step or element or group of steps or elements.

By “corresponds to” or “corresponding to” is meant a polynucleotide (a) having a nucleotide sequence that is substantially identical or complementary to all or a portion of a reference polynucleotide sequence or (b) encoding an amino acid sequence identical to an amino acid sequence in a peptide or protein. This phrase also includes within its scope a peptide or polypeptide having an amino acid sequence that is substantially identical to a sequence of amino acids in a reference peptide or protein.

By “derivative” is meant a polypeptide that has been derived from the basic sequence by modification, for example by conjugation or complexing with other chemical moieties or by post-translational modification techniques as would be understood in the art. The term “derivative” also includes within its scope alterations that have been made to a parent sequence including additions, or deletions that provide for functionally equivalent molecules. Accordingly, the term derivative encompasses molecules that will have at least one activity selected from the group consisting of cell differentiation, cell proliferation and tumorigenesis.

“Homology” refers to the percentage number of amino acids that are identical or constitute conservative substitutions as defined in Table B infra. Homology may be determined using sequence comparison programs such as GAP (Deveraux et al. 1984, Nucleic Acids Research 12, 387-395). In this way, sequences of a similar or substantially different length to those cited herein might be compared by insertion of gaps into the alignment, such gaps being determined, for example, by the comparison algorithm used by GAP.

“Hybridisation” is used herein to denote the pairing of complementary nucleotide sequences to produce a DNA-DNA hybrid or a DNA-RNA hybrid. Complementary base sequences are those sequences that are related by the base-pairing rules. In DNA, A pairs with T and C pairs with G. In RNA U pairs with A and C pairs with G. In this regard, the terms “match” and “mismatch” as used herein refer to the hybridisation potential of paired nucleotides in complementary nucleic acid strands. Matched nucleotides hybridise efficiently, such as the classical A-T and G-C base pair mentioned above. Mismatches are other combinations of nucleotides that do not hybridise efficiently.

Reference herein to “immuno-interactive” includes reference to any interaction, reaction, or other form of association between molecules and in particular where one of the molecules is, or mimics, a component of the immune system.

By “immuno-interactive fragment” is meant a fragment of the polypeptide set forth in SEQ ID NO: 2 which fragment elicits an immune response, including the production of elements that specifically bind to said polypeptide, or variant or derivative thereof. As used herein, the term “immuno-interactive fragment” includes deletion mutants and small peptides, for example of at least six, preferably at least 8 and more preferably at least 12, even more preferably at least 15, even more preferably at least 18 and still even more preferably at least 20 contiguous amino acids, which comprise antigenic determinants or epitopes. Several such fragments may be joined together.

By “isolated” is meant material that is substantially or essentially free from components that normally accompany it in its native state. For example, an “isolated polynucleotide”, as used herein, refers to a polynucleotide, which has been purified from the sequences which flank it in a naturally occurring state, e.g., a DNA fragment which has been removed from the sequences which are normally adjacent to the fragment.

By “modulating” is meant increasing or decreasing, either directly or indirectly, the level and/or functional activity of a target molecule. For example, an agent may indirectly modulate the said level/activity by interacting with a molecule other than the target molecule. In this regard, indirect modulation of a gene encoding a target polypeptide includes within its scope modulation of the expression of a first nucleic acid molecule, wherein an expression product of the first nucleic acid molecule modulates the expression of a nucleic acid molecule encoding the target polypeptide.

By “obtained from” is meant that a sample such as, for example, a nucleic acid extract or polypeptide extract is isolated from, or derived from, a particular source of the host. For example, the extract may be obtained from a tissue or a biological fluid isolated directly from the host.

The term “oligonucleotide” as used herein refers to a polymer composed of a multiplicity of nucleotide units (deoxyribonucleotides or ribonucleotides, or related structural variants or synthetic analogues thereof) linked via phosphodiester bonds (or related structural variants or synthetic analogues thereof). Thus, while the term “oligonucleotide” typically refers to a nucleotide polymer in which the nucleotides and linkages between them are naturally occurring, it will be understood that the term also includes within its scope various analogues including, but not restricted to, peptide nucleic acids (PNAs), phosphoramidates, phosphorothioates, methyl phosphonates, 2-O-methyl ribonucleic acids, and the like. The exact size of the molecule may vary depending on the particular application. An oligonucleotide is typically rather short in length, generally from about 10 to 30 nucleotides, but the term can refer to molecules of any length, although the term “polynucleotide” or “nucleic acid” is typically used for large oligonucleotides.

By “operably linked” is meant that transcriptional and translational regulatory nucleic acids are positioned relative to a polypeptide-encoding polynucleotide in such a manner that the polynucleotide is transcribed and the polypeptide is translated.

The term “patient” refers to patients of human or other mammal and includes any individual it is desired to examine or treat using the methods of the invention. However, it will be understood that “patient” does not imply that symptoms are present. Suitable mammals that fall within the scope of the invention include, but are not restricted to, primates, livestock animals (e.g. sheep, cows, horses, donkeys, pigs), laboratory test animals (e.g. rabbits, mice, rats, guinea pigs, hamsters), companion animals (e.g. cats, dogs) and captive wild animals (e.g. foxes, deer, dingoes).

By “pharmaceutically-acceptable carrier” is meant a solid or liquid filler, diluent or encapsulating substance that may be safely used in topical or systemic administration.

The term “polynucleotide” or “nucleic acid” as used herein designates mRNA, RNA, cRNA, cDNA or DNA. The term typically refers to oligonucleotides greater than 30 nucleotides in length. Polynucleotide sequences are understood to encompass complementary strands as well as alternative backbones described herein.

The terms “polynucleotide variant” and “variant” refer to polynucleotides displaying substantial sequence identity with a reference polynucleotide sequence or polynucleotides that hybridise with a reference sequence under stringent conditions that are defined hereinafter. These terms also encompasses polynucleotides in which one or more nucleotides have been added or deleted, or replaced with different nucleotides. In this regard, it is well understood in the art that certain alterations inclusive of mutations, additions, deletions and substitutions can be made to a reference polynucleotide whereby the altered polynucleotide retains the biological function or activity of the reference polynucleotide. The terms “polynucleotide variant” and “variant” also include naturally occurring allelic variants.

“Polypeptide”, “peptide” and “protein” are used interchangeably herein to refer to a polymer of amino acid residues and to variants and synthetic analogues of the same. Thus, these terms apply to amino acid polymers in which one or more amino acid residues is a synthetic non-naturally occurring amino acid, such as a chemical analogue of a corresponding naturally occurring amino acid, as well as to naturally-occurring amino acid polymers.

The term “polypeptide variant” refers to polypeptides in which one or more amino acids have been replaced by different amino acids. It is well understood in the art that some amino acids may be changed to others with broadly similar properties without changing the nature of the activity of the polypeptide (conservative substitutions) as described hereinafter. Accordingly, polypeptide variants as used herein encompass polypeptides that have an activity selected from the group consisting of cell differentiation, cell proliferation and tumorigenesis.

By “primer” is meant an oligonucleotide which, when paired with a strand of DNA, is capable of initiating the synthesis of a primer extension product in the presence of a suitable polymerising agent. The primer is preferably single-stranded for maximum efficiency in amplification but may alternatively be double-stranded. A primer must be sufficiently long to prime the synthesis of extension products in the presence of the polymerisation agent. The length of the primer depends on many factors, including application, temperature to be employed, template reaction conditions, other reagents, and source of primers. For example, depending on the complexity of the target sequence, the oligonucleotide primer typically contains 15 to 35 or more nucleotides, although it may contain fewer nucleotides. Primers can be large polynucleotides, such as from about 200 nucleotides to several kilobases or more. Primers may be selected to be “substantially complementary” to the sequence on the template to which it is designed to hybridise and serve as a site for the initiation of synthesis. By “substantially complementary”, it is meant that the primer is sufficiently complementary to hybridise with a target nucleotide sequence. Preferably, the primer contains no mismatches with the template to which it is designed to hybridise but this is not essential. For example, non-complementary nucleotides may be attached to the 5′ end of the primer, with the remainder of the primer sequence being complementary to the template. Alternatively, non-complementary nucleotides or a stretch of non-complementary nucleotides can be interspersed into a primer, provided that the primer sequence has sufficient complementarity with the sequence of the template to hybridise therewith and thereby form a template for synthesis of the extension product of the primer.

“Probe” refers to a molecule that binds to a specific sequence or sub-sequence or other moiety of another molecule. Unless otherwise indicated, the term “probe” typically refers to a polynucleotide probe that binds to another nucleic acid, often called the “target nucleic acid”, through complementary base pairing. Probes may bind target nucleic acids lacking complete sequence complementarity with the probe, depending on the stringency of the hybridisation conditions. Probes can be labelled directly or indirectly.

The term “recombinant polynucleotide” as used herein refers to a polynucleotide formed in vitro by the manipulation of nucleic acid into a form not normally found in nature. For example, the recombinant polynucleotide may be in the form of an expression vector. Generally, such expression vectors include transcriptional and translational regulatory nucleic acid operably linked to the nucleotide sequence.

By “recombinant polypeptide” is meant a polypeptide made using recombinant techniques, i.e., through the expression of a recombinant polynucleotide.

By “reporter molecule” as used in the present specification is meant a molecule that, by its chemical nature, provides an analytically identifiable signal that allows the detection of a complex comprising an antigen-binding molecule and its target antigen. The term “reporter molecule” also extends to use of cell agglutination or inhibition of agglutination such as red blood cells on latex beads, and the like.

Terms used to describe sequence relationships between two or more polynucleotides or polypeptides include “reference sequence”, “comparison window”, “sequence identity”, “percentage of sequence identity” and “substantial identity”. A “reference sequence” is at least 12 but frequently 15 to 18 and often at least 25 monomer units, inclusive of nucleotides and amino acid residues, in length. Because two polynucleotides may each comprise (1) a sequence (i.e., only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of at least 6 contiguous positions, usually about 50 to about 100, more usually about 100 to about 150 in which a sequence is compared to a reference sequence of the same number of contiguous positions after the two sequences are optimally aligned. The comparison window may comprise additions or deletions (i.e., gaps) of about 20% or less as compared to the reference sequence (which does not comprise additions or deletions) for optimal alignment of the two sequences. Optimal alignment of sequences for aligning a comparison window may be conducted by computerised implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e., resulting in the highest percentage homology over the comparison window) generated by any of the various methods selected. Reference also may be made to the BLAST family of programs as for example disclosed by Altschul et al., 1997, Nucl. Acids Res. 25:3389. A detailed discussion of sequence analysis can be found in Unit 19.3 of Ausubel et al., “Current Protocols in Molecular Biology”, John Wiley & Sons Inc, 1994-1998, Chapter 15.

The term “sequence identity” as used herein refers to the extent that sequences are identical on a nucleotide-by-nucleotide basis or an amino acid-by-amino acid basis over a window of comparison. Thus, a “percentage of sequence identity” is calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. For the purposes of the present invention, “sequence identity” will be understood to mean the “match percentage” calculated by the DNASIS computer program (Version 2.5 for windows; available from Hitachi Software engineering Co., Ltd., South San Francisco, Calif., USA) using standard defaults as used in the reference manual accompanying the software.

“Stringency” as used herein, refers to the temperature and ionic strength conditions, and presence or absence of certain organic solvents, during hybridisation and washing procedures. The higher the stringency, the higher will be the degree of complementarity between immobilised target nucleotide sequences and the labelled probe polynucleotide sequences that remain hybridised to the target after washing.

“Stringent conditions” refers to temperature and ionic conditions under which only nucleotide sequences having a high frequency of complementary bases will hybridise. The stringency required is nucleotide sequence dependent and depends upon the various components present during hybridisation and subsequent washes, and the time allowed for these processes. Generally, in order to maximise the hybridisation rate, non-stringent hybridisation conditions are selected; about 20 to 25° C. lower than the thermal melting point (Tₘ). The Tₘ is the temperature at which 50% of specific target sequence hybridises to a perfectly complementary probe in solution at a defined ionic strength and pH. Generally, in order to require at least about 85% nucleotide complementarity of hybridised sequences, highly stringent washing conditions are selected to be about 5 to 15° C. lower than the Tₘ. In order to require at least about 70% nucleotide complementarity of hybridised sequences, moderately stringent washing conditions are selected to be about 15 to 30° C. lower than the Tₘ. Highly permissive (low stringency) washing conditions may be as low as 50° C. below the Tₘ, allowing a high level of mismatching between hybridised sequences. Those skilled in the art will recognise that other physical and chemical parameters in the hybridisation and wash stages can also be altered to affect the outcome of a detectable hybridisation signal from a specific level of homology between target and probe sequences. Other examples of stringency conditions are described in section 3.3.

By “therapeutically effective amount”, in the context of treating a cancer or tumour, is meant the administration of that amount of a polypeptide, fragment, variant, derivative or modulatory agent that modulates the expression of OKL38 to an individual in need of such treatment, either in a single dose or as part of a series, that is effective for treatment or prophylaxis of that cancer or tumour. The effective amount will vary depending upon the health and physical condition of the individual to be treated, the taxonomic group of individual to be treated, the formulation of the composition, the assessment of the medical situation, and other relevant factors. It is expected that the amount will fall in a relatively broad range that can be determined through routine trials.

By “vector” is meant a nucleic acid molecule, preferably a DNA molecule derived, for example, from a plasmid, bacteriophage, or plant virus, into which a nucleic acid sequence may be inserted or cloned. A vector preferably contains one or more unique restriction sites and may be capable of autonomous replication in a defined host cell including a target cell or tissue or a progenitor cell or tissue thereof, or be integrable with the genome of the defined host such that the cloned sequence is reproducible. Accordingly, the vector may be an autonomously replicating vector, i.e., a vector that exists as an extrachromosomal entity, the replication of which is independent of chromosomal replication, e.g., a linear or closed circular plasmid, an extrachromosomal element, a minichromosome, or an artificial chromosome. The vector may contain any means for assuring self-replication. Alternatively, the vector may be one which, when introduced into the host cell, is integrated into the genome and replicated together with the chromosome(s) into which it has been integrated. A vector system may comprise a single vector or plasmid, two or more vectors or plasmids, which together contain the total DNA to be introduced into the genome of the host cell, or a transposon. The choice of the vector will typically depend on the compatibility of the vector with the host cell into which the vector is to be introduced. The vector may also include a selection marker such as an antibiotic resistance gene that can be used for selection of suitable transformants. Examples of such resistance genes are well known to those of skill in the art.

As used herein, underscoring or italicising the name of a gene shall indicate the gene, in contrast to its protein product, which is indicated in the absence of any underscoring or italicising. For example, “OKL38” shall mean the OKL38 gene or cDNA sequence, whereas “OKL38” shall indicate the protein product of the “OKL38” gene.

2. Isolated Polypeptides, Biologically Active Fragments, Polypeptide Variants and Derivatives

2.1 Polypeptides of the Invention

In work leading up to the present invention, a novel human gene, designated OKL38, was discovered that modulates differentiation and proliferation of epithelial cells, and particularly of mammary epithelial cells. The deduced polypeptide sequence of human OKL38 comprises 317 amino acids as set forth in SEQ ID NO: 2 and its predicted molecular weight is 34.5 kDa. The endogenous OKL38 polypeptide is predicted, by computational analysis, to be a soluble cytoplasmic protein.

2.2 Biologically Active Fragments

Biologically active fragments may be produced according to any suitable procedure known in the art. For example, a suitable method may include first producing a fragment of said isolated polypeptide and then testing the fragment for the appropriate biological activity. In one embodiment, biological activity of the fragment is tested by introducing a fragment of the polypeptide or a polynucleotide from which the fragment can be translated into a cell, and detecting modulation of at least one activity selected from the group consisting of cell differentiation, cell proliferation and tumorigenesis, which is indicative of said fragment being a said biologically active fragment. Preferably, said fragment promotes cell differentiation. Suitably said fragment inhibits cell proliferation and/or tumorigenesis. The cell is preferably selected from the group consisting of a mammary cell, a liver cell, a kidney cell, a testicular cell, a bladder cell and a lung cell. In a preferred embodiment, the cell is mammary cell.

The invention also extends to biological fragments of the above polypeptides, which can elicit an immune response in an animal and preferably in a heterologous animal from which the polypeptide is obtained. For example exemplary polypeptide fragments of 8 residues in length, which could elicit an immune response, include but are not limited to residues 1-8, 9-16, 17-24, 25-32, 33-40, 41-48, 49-56, 57-64, 65-72, 73-80, 81-88, 89-96, 97-104, 105-112, 113-120, 121-128, 129-136, 137-144, 145-152, 153-160, 161-168, 169-176, 177-184, 185-192, 193-200, 201-208, 209-216, 217-224, 225-232, 223-240, 241-248, 249-256, 257-264, 265-272, 273-280, 281-288, 289-296, 297-304, 305-312 and 310-317 of SEQ ID NO: 2.

2.3 Polypeptide Variants

The invention also contemplates polypeptide variants of the polypeptide described to above wherein said variants modulate at least one activity selected from the group consisting of cell differentiation, cell proliferation and tumorigenesis. The variants may be naturally occurring and may be obtained, for example from mammals including, but not limited to, rodents, non-human primates, ovines, bovines, ruminants, lagomorphs, porcines, caprines, equines, canines, felines and aves. Alternatively, variant polypeptides may be produced by recombinant, synthetic or other techniques known to persons of skill in the art. Suitable methods of producing polypeptide variants include, for example, replacing at least one amino acid of a parent polypeptide comprising the sequence set forth in any one of SEQ ID NO: 2, or a biologically active fragment thereof, with a different amino acid to produce a modified polypeptide, introducing said polypeptide or a polynucleotide from which the fragment can be translated into a cell, and detecting modulation of at least one activity selected from the group consisting of cell differentiation, cell proliferation, and tumorigenesis, which is indicative of the modified polypeptide being a said polypeptide variant.

Any suitable assay for detecting, measuring or otherwise determining modulation of cell proliferation, cell differentiation and tumorigenesis is contemplated by the present invention. Assays of these types are known to persons of skill in the art. Typically, cell number is determined, directly, by microscopic or electronic enumeration, or indirectly, by the use of chromogenic dyes, incorporation of radioactive precursors or measurement of metabolic activity of cellular enzymes. An exemplary cell proliferation assay comprises culturing cells in the presence or absence of a test compound, and detecting cell proliferation by, for example, measuring incorporation of tritiated thymidine or by colorimetric assay based on the metabolic breakdown of 3-(4,5-dimethylthiazol-2-yl)-2,5-diphenyl tetrazolium bromide (MTT) (Mosman, 1983, J. Immunol. Meth. 65: 55-63).

Cell differentiation may be assessed in several different ways. One such method is by measuring cell phenotypes. The phenotypes of cells and any phenotypic changes can be evaluated by flow cytometry after immunofluorescent staining using monoclonal antibodies that will bind membrane proteins characteristic of various cell types. A second means of assessing cell differentiation is by measuring cell function. This may be done biochemically, by measuring the expression of enzymes, mRNAs, genes, proteins, or other metabolites within the cell, or secreted from the cell. Bioassays may also be used to measure functional cell differentiation.

Cells including, but not restricted to, mammary cells, liver cells, kidney cells, testicular cells, bladder cells and lung cells, express a variety of cell surface molecules which can be detected with monoclonal antibodies or polyclonal antisera or other antigen-binding molecules. Cells that have undergone differentiation can also be enumerated by staining for the presence of characteristic cell surface proteins by direct immunofluorescence in fixed smears of cultured cells.

Cancer or tumour markers are known for a variety of cell or tissue types. Cells or tissues expressing cancer or tumour markers may be detected using monoclonal antibodies, polyclonal antisera or other antigen-binding molecules that are immuno-interactive with these markers or by using nucleic acid analysis techniques, including, for example, detecting the level or presence of marker-encoding polynucleotides.

In general, variants will have at least 50%, more suitably at least 70%, preferably at least 80%, more preferably at least 90% and even more preferably at least 95% homology to a polypeptide as for example shown in SEQ ID NO: 2, or fragments thereof. It is preferred that variants display at least 50%, more suitably at least 70%, preferably at least 80%, more preferably at least 90% and still more preferably at least 90% sequence identity to a polypeptide as for example shown in SEQ ID NO: 2, or fragments thereof. In this respect, the window of comparison spans at least about 8 amino acids, preferably at least about 20 amino acids, more preferably at least about 50 amino acids, more preferably at least about 100 amino acid and even more preferably at least about 200 amino acids.

Suitable variants can be obtained from any suitable animal. An example of a polypeptide variant according to the invention is a murine polypeptide whose sequence is set forth in SEQ ID NO: 8. Preferably, the variants are obtained from a mammal by methods as for example described in Section 3.2 infra.

2.4 Methods of Producing Polypeptide Variants

2.4.1 Mutagenesis

Polypeptide variants according to the invention can be identified either rationally, or via established methods of mutagenesis (see, for example, Watson, J. D. et al., “MOLECULAR BIOLOGY OF THE GENE”, Fourth Edition, Benjamin/Cummings, Menlo Park, Calif., 1987). Significantly, a random mutagenesis approach requires no a priori information about the gene sequence that is to be mutated. This approach has the advantage that it assesses the desirability of a particular mutant based on its function, and thus does not require an understanding of how or why the resultant mutant protein has adopted a particular conformation. Indeed, the random mutation of target gene sequences has been one approach used to obtain mutant proteins having desired characteristics (Leatherbarrow, R. 1986, J. Prot. Eng. 1: 7-16; Knowles, J. R., 1987, Science 236: 1252-1258; Shaw, W. V., 1987, Biochem. J. 246: 1-17; Gerit, J. A. 1987, Chem. Rev. 87: 1079-1105). Alternatively, where a particular sequence alteration is desired, methods of site-directed mutagenesis can be employed. Thus, such methods may be used to selectively alter only those amino acids of the protein that are believed to be important (Craik, C. S., 1985, Science 228: 291-297; Cronin, et al., 1988, Biochem. 27: 4572-4579; Wilks, et al., 1988, Science 242: 1541-1544).

Variant peptides or polypeptides, resulting from rational or established methods of mutagenesis or from combinatorial chemistries as hereinafter described, may comprise conservative amino acid substitutions. Exemplary conservative substitutions in a polypeptide or polypeptide fragment according to the invention may be made according to the following table:

TABLE A

| Original Residue | Exemplary Substitutions |
|---|---|
| Ala | Ser |
| Arg | Lys |
| Asn | Gln, His |
| Asp | Glu |
| Cys | Ser |
| Gln | Asn |
| Glu | Asp |
| Gly | Pro |
| His | Asn, Gln |
| Ile | Leu, Val |
| Leu | Ile, Val |
| Lys | Arg, Gln, Glu |
| Met | Leu, Ile, |
| Phe | Met, Leu, Tyr |
| Ser | Thr |
| Thr | Ser |
| Trp | Tyr |
| Tyr | Trp, Phe |
| Val | Ile, Leu |

Substantial changes in function are made by selecting substitutions that are less conservative than those shown in TABLE A. Other replacements would be non-conservative substitutions and relatively fewer of these may be tolerated. Generally, the substitutions which are likely to produce the greatest changes in a polypeptide's properties are those in which (a) a hydrophilic residue (e.g., Ser or Thr) is substituted for, or by, a hydrophobic residue (e.g., Ala, Leu, Ile, Phe or Val); (b) a cysteine or proline is substituted for, or by, any other residue; (c) a residue having an electropositive side chain (e.g., Arg, His or Lys) is substituted for, or by, an electronegative residue (e.g., Glu or Asp) or (d) a residue having a bulky side chain (e.g., Phe or Trp) is substituted for, or by, one having a smaller side chain (e.g., Ala, Ser) or no side chain (e.g., Gly).

What constitutes suitable variants may be determined by conventional techniques. For example, nucleic acids encoding a polypeptide according to SEQ ID NO: 2 can be mutated using either random mutagenesis for example using transposon mutagenesis, or site-directed mutagenesis as described, for example, in Section 3.2 infra.

2.4.2 Peptide Libraries Produced by Combinatorial Chemistry

A number of facile combinatorial technologies can be utilised to synthesise molecular libraries of immense diversity. In the present case, variants of a polypeptide, or preferably a polypeptide fragment according to the invention, can be synthesised using such technologies. These polypeptide fragments may be immuno-interactive or may bind to a binding partner involved in modulation of an activity selected from the group consisting of cell differentiation, cell proliferation and tumorigenesis. Variants can be screened subsequently using, for example, the methods described in Section 2.3.

Preferably, soluble synthetic peptide combinatorial libraries (SPCLs) are produced which offer the advantage of working with free peptides in solution, thus permitting adjustment of peptide concentration to accommodate a particular assay system. SPCLs are suitably prepared as hexamers. In this regard, a majority of binding sites is known to involve four to six residues. Cysteine is preferably excluded from the mixture positions to avoid the formation of disulphides and more difficult-to-define polymers. Exemplary methods of producing SPCLs are disclosed by Houghten et al. (1991, Nature 354: 84-86; 1992, BioTechniques 13: 412-421), Appel et al. (1992, Immunomethods 1: 17-23), and Pinilla et al. (1992, BioTechniques 13: 901-905; 1993, Gene 128: 71-76).

Preparation of combinatorial synthetic peptide libraries may employ either t-butyloxycarbonyl (t-Boc) or 9-fluorenylmethyloxycarbonyl (Fmoc) chemistries (see Chapter 9.1, of Coligan et al., supra; Stewart and Young, 1984, Solid Phase Peptide Synthesis, 2nd ed. Pierce Chemical Co., Rockford, Ill.; and Atherton and Sheppard, 1989, Solid Phase Peptide Synthesis: A Practical Approach. IRL Press, Oxford) preferably, but not exclusively, using one of two different approaches. The first of these approaches, suitably termed the “split-process-recombine” or “split synthesis” method, was described first by Furka et al. (1988, 14th Int. Congr. Biochem., Prague, Czechoslovakia 5: 47; 1991, Int. J. Pept. Protein Res. 37: 487-493) and Lam et al. (1991, Nature 354: 82-84), and reviewed later by Eichler et al. (1995, Medicinal Research Reviews 15(6): 481-496) and Balkenhohl et al. (1996, Angew. Chem. Int. Ed. Engl. 35: 2288-2337). Briefly, the split synthesis method involves dividing a plurality of solid supports such as polymer beads into n equal fractions representative of the number of available amino acids for each step of the synthesis (e.g., 20 L-amino acids), coupling a single respective amino acid to each polymer bead of a corresponding fraction, and then thoroughly mixing the polymer beads of all the fractions together. This process is repeated for a total of x cycles to produce a stochastic collection of up to N^{x} different compounds. The peptide library so produced may be screened for example with a suitably labelled antigen-binding molecule that binds specifically to a polypeptide according to SEQ ID NO: 2. Upon detection, some of the positive beads are selected for sequencing to identify the active peptide. Such peptide may be subsequently cleaved from the beads, and assayed using the same antigen-binding molecule or other binding partner to identify the most active peptide sequence.

The second approach, the chemical ratio method, prepares mixed peptide resins using a specific ratio of amino acids empirically defined to give equimolar incorporation of each amino acid at each coupling step. Each resin bead contains a mixture of peptides. Approximate equimolar representation can be confirmed by amino acid analysis (Dooley and Houghten, 1993, Proc. Natl. Acad. Sci. U.S.A. 90: 10811-10815; Eichler and Houghten, 1993, Biochemistry 32: 11035-11041). Preferably, the synthetic peptide library is produced on polyethylene rods, or pins, as a solid support, as for example disclosed by Geysen et al. (1986, Mol. Immunol. 23: 709-715). An exemplary peptide library of this type may consist of octapeptides in which the third and fourth positions represent defined amino acids selected from natural and non-natural amino acids, and in which the remaining six positions represent a randomised mixture of amino acids. This peptide library can be represented by the formula Ac-X₁X₂O₁O₂X₃X₄X₅X₆-Sₛ, where O₁ and O₂ are each defined amino acids, X₁₋₆ are a randomised mixture of amino acids and Sₛ is the solid support. Peptide mixtures remain on the pins when assayed against a soluble receptor molecule. For example, the peptide library of Geysen (1986, Immun. Today 6: 364-369; and Geysen et al., Ibid), comprising for example dipeptides, is first screened for the ability to bind to a target molecule. The most active dipeptides are then selected for an additional round of testing comprising linking, to the starting dipeptide, an additional residue (or by internally modifying the components of the original starting dipeptide) and then screening this set of candidates for the desired activity. The best tripeptide is used as the basis of a tetrapeptide and so on until an optimised sequence up to an octapeptide with the desired properties has been determined.

2.4.3 Alanine Scanning Mutagenesis

In one embodiment, the invention herein utilises a systematic analysis of a polypeptide or polypeptide fragment according to the invention to determine the residues in the polypeptide or fragment that are involved in the interaction with a specific binding partner such as an antigen binding molecule or a partner involved in modulation of an activity selected from the group consisting of cell differentiation, cell proliferation and tumorigenesis. Such analysis is conveniently performed using recombinant DNA technology. In general, a DNA sequence encoding the polypeptide or fragment is cloned and manipulated so that it may be expressed in a convenient host. DNA encoding the polypeptide or fragment can be obtained from a genomic library, from cDNA derived from mRNA in cells expressing the said polypeptide or fragment, or by synthetically constructing the DNA sequence (Sambrook et al., supra; Ausubel et al., supra).

The wild-type DNA encoding the polypeptide or fragment is then inserted into an appropriate plasmid or vector as described herein. In particular, prokaryotes are preferred for cloning and expressing DNA sequences to produce variants of the polypeptide or fragment. For example, E. coli K12 strain 294 (ATCC No. 31446) may be used, as well as E. coli B, E. coli X1776 (ATCC No. 31537), and E. coli c600 and c600hfl, and E. coli W3110 (F⁻, γ⁻, prototrophic, ATCC No. 27325), bacilli such as Bacillus subtilis, and other enterobacteriaceae such as Salmonella typhimurium or Serratia marcescens, and various Pseudomonas species. A preferred prokaryote is E. coli W3110 (ATCC 27325).

Once the polypeptide or fragment is cloned, site-specific mutagenesis as for example described by Carter et al. (1986, Nucl. Acids. Res., 13: 4331) or by Zoller et al. (1987, Nucl. Acids Res., 10: 6487), cassette mutagenesis as for example described by Wells et al. (1985, Gene, 34: 315), restriction selection mutagenesis as for example described by Wells et al. (1986, Philos. Trans. R. Soc. London SerA, 317: 415), or other known techniques may be performed on the cloned DNA to produce the variant DNA that encodes for the changes in amino acid sequence defined by the residues being substituted. When operably linked to an appropriate expression vector, variants are obtained. In some cases, recovery of the variant may be facilitated by expressing and secreting such molecules from the expression host by use of an appropriate signal sequence operably linked to the DNA sequence encoding the variant. Such methods are well known to those skilled in the art. Of course, other methods may be employed to produce such polypeptides or fragments such as the in vitro chemical synthesis of the desired polypeptide variant (Barany et al. In The Peptides, eds. E. Gross and J. Meienhofer (Academic Press: N.Y. 1979), Vol. 2, pp. 3-254).

Once the different variants are produced, they are contacted with an OKL38-specific binding partner (e.g., an antigen-binding molecule) and the interaction, if any, between this binding partner and each variant is determined. These activities are compared to the activity of the parent polypeptide or fragment with the same binding partner molecule to determine which of the amino acid residues in the active domain are involved in the interaction with the binding partner. The scanning amino acid used in such an analysis may be any different amino acid from that substituted, i.e., any of the 19 other naturally occurring amino acids.

The interaction between the OKL38-specific binding partner, and parent and variant, respectively, can be measured by any convenient assay as for example described herein. While any number of analytical measurements may be used to compare activities, a convenient one for binding of the OKL38-specific binding partner is the dissociation constant K_{d} of the complex formed between the variant and that binding partner as compared to the K_{d} for the parent polypeptide or fragment. Generally, a two-fold increase or decrease in K_{d} per analogous residue substituted by the substitution indicates that the substituted residue(s) is active in the interaction of the parent polypeptide or fragment with the target-binding partner.

When a suspected or known active amino acid residue is subjected to scanning amino acid analysis, the amino acid residues immediately adjacent thereto should be scanned. Three residue-substituted polypeptides can be made. One contains a scanning amino acid, preferably alanine, at position N that is the suspected or known active amino acid. The two others contain the scanning amino acid at position N+1 and N−1. If each substituted polypeptide or fragment causes a greater than about two-fold effect on K_{d} for the receptor, the scanning amino acid is substituted at position N+2 and N−2. This is repeated until at least one, and preferably four, residues are identified in each direction which have less than about a two-fold effect on K_{d} or either of the ends of the parent polypeptide or fragment are reached. In this manner, one or more amino acids along a continuous amino acid sequence that are involved in the interaction with the particular binding partner molecule can be identified.

The active amino acid residue identified by amino acid scan is typically one that contacts the OKL38-specific binding partner directly. However, active amino acids may also indirectly contact the binding partner through salt bridges formed with other residues or small molecules such as H₂O or ionic species such as Na⁺, Ca⁺², Mg⁺², or Zn⁺².

In some cases, the substitution of a scanning amino acid at one or more residues results in a residue-substituted polypeptide which is not expressed at levels that allow for the isolation of quantities sufficient to carry out analysis of its activity with the OKL38-specific binding partner. In such cases, a different scanning amino acid, preferably an isosteric amino acid, can be used.

Among the preferred scanning amino acids are relatively small, neutral amino acids. Such amino acids include alanine, glycine, serine, and cysteine. Alanine is the preferred scanning amino acid among this group because it eliminates the side-chain beyond the beta-carbon and is less likely to alter the main-chain conformation of the variant. Alanine is also preferred because it is the most common amino acid. Further, it is frequently found in both buried and exposed positions (Creighton, The Proteins, W. H. Freeman & Co., N.Y.; Chothia, 1976, J. Mol. Biol., 150: 1). If alanine substitution does not yield adequate amounts of variant, an isosteric amino acid can be used. Alternatively, the following amino acids in decreasing order of preference may be used: Ser, Asn, and Leu.

Once the active amino acid residues are identified, isosteric amino acids may be substituted. Such isosteric substitutions need not occur in all instances and may be performed before any active amino acid is identified. Such isosteric amino acid substitution is performed to minimise the potential disruptive effects on conformation that some substitutions can cause. Isosteric amino acids are shown in the table below:

TABLE B

| Polypeptide Amino Acid | Isosteric Scanning Amino Acid |
|---|---|
| Ala (A) | Ser, Gly |
| Glu (E) | Gln, Asp |
| Gln (Q) | Asn, Glu |
| Asp (D) | Asn, Glu |
| Asn (N) | Ala, Asp |
| Leu (L) | Met, Ile |
| Gly (G) | Pro, Ala |
| Lys (K) | Met, Arg |
| Ser (S) | Thr, Ala |
| Val (V) | Ile, Thr |
| Arg (R) | Lys, Met, Asn |
| Thr (T) | Ser, Val |
| Pro (P) | Gly |
| Ile (I) | Met, Leu, Val |
| Met (M) | Ile, Leu |
| Phe (F) | Tyr |
| Tyr (Y) | Phe |
| Cys (C) | Ser, Ala |
| Trp (W) | Phe |
| His (H) | Asn, Gln |

The method herein can be used to detect active amino acid residues within different domains of a polypeptide or fragment according to the invention. Once this identification is made various modifications to the parent polypeptide or fragment may be made to modify the interaction between the parent polypeptide or fragment and a specific binding partner.

2.4.4 Polypeptide or Peptide Libraries Produced by Phage Display

The identification of variants can also be facilitated through the use of a phage (or phagemid) display protein ligand screening system as for example described by Lowman, et al. (1991, Biochem. 30: 10832-10838), Markland, et al. (1991, Gene 109: 13-19), Roberts, et al. (1992, Proc. Natl. Acad. Sci. (U.S.A.) 89: 2429-2433), Smith, G. P. (1985, Science 228: 1315-1317), Smith, et al. (1990, Science 248: 1126-1128) and Lardner et al. (U.S. Pat. No. 5,223,409). In general, this method involves expressing a fusion protein in which the desired protein ligand is fused to the N-terminus of a viral coat protein (such as the M13 Gene III coat protein, or a lambda coat protein).

In one embodiment, a library of phage is engineered to display novel peptides within the phage coat protein sequences. Novel peptide sequences are generated by random mutagenesis of gene fragments encoding a polypeptide of the invention or biologically active fragment using error-prone PCR, or by in vivo mutation by E. coli mutator cells. The novel peptides displayed on the surface of the phage are placed in contact, with an OKL38-specific binding partner molecule. Phage that display coat protein having peptides that are capable of binding to a binding partner are immobilized by such treatment, whereas all other phage can be washed away. After the removal of unbound phage, the bound phage can be amplified, and the DNA encoding their coat proteins can be sequenced. In this manner, the amino acid sequence of the embedded peptide or polypeptide can be deduced.

In more detail, the method involves (a) constructing a replicable expression vector comprising a first gene encoding a polypeptide or fragment of the invention, a second gene encoding at least a portion of a natural or wild-type phage coat protein wherein the first and second genes are heterologous, and a transcription regulatory element operably linked to the first and second genes, thereby forming a gene fusion encoding a fusion protein; (b) mutating the vector at one or more selected positions within the first gene thereby forming a family of related plasmids; (c) transforming suitable host cells with the plasmids; (d) infecting the transformed host cells with a helper phage having a gene encoding the phage coat protein; (e) culturing the transformed infected host cells under conditions suitable for forming recombinant phagemid particles containing at least a portion of the plasmid and capable of transforming the host, the conditions adjusted so that no more than a minor amount of phagemid particles display more than one copy of the fusion protein on the surface of the particle; (f) contacting the phagemid particles with an OKL38-specific binding partner that binds to the parent polypeptide or fragment so that at least a portion of the phagemid particles bind to the binding partner; and (g) separating the phagemid particles that bind from those that do not. Preferably, the method further comprises transforming suitable host cells with recombinant phagemid particles that bind to the OKL38-specific binding partner and repeating steps (d) through (g) one or more times.

Preferably in this method the plasmid is under tight control of the transcription regulatory element, and the culturing conditions are adjusted so that the amount or number of phagemid particles displaying more than one copy of the fusion protein on the surface of the particle is less than about 1%. Also, preferably, the amount of phagemid particles displaying more than one copy of the fusion protein is less than 10% of the amount of phagemid particles displaying a single copy of the fusion protein. Most preferably, the amount is less than 20%.

Typically in this method, the expression vector will further contain a secretory signal sequence fused to the DNA encoding each subunit of the polypeptide and the transcription regulatory element will be a promoter system. Preferred promoter systems are selected from lac Z, λ_{PL}, tac, T7 polymerase, tryptophan, and alkaline phosphatase promoters and combinations thereof. Also, normally the method will employ a helper phage selected from M13K07, M13R408, M13-VCS, and Phi X 174. The preferred helper phage is M13K07, and the preferred coat protein is the M13 Phage gene III coat protein. The preferred host is E. coli, and protease-deficient strains of E. coli.

Repeated cycles of variant selection are used to select for higher and higher affinity binding by the phagemid selection of multiple amino acid changes that are selected by multiple selection cycles. Following a first round of phagemid selection, involving a first region or selection of amino acids in the ligand polypeptide, additional rounds of phagemid selection in other regions or amino acids of the ligand polypeptide are conducted. The cycles of phagemid selection are repeated until the desired affinity properties of the ligand polypeptide are achieved.

It will be appreciated that the amino acid residues that form the binding domain of the polypeptide or fragment may not be sequentially linked and may reside on different subunits of the polypeptide or fragment. That is, the binding domain tracks with the particular secondary structure at the binding site and not the primary structure. Thus, generally, mutations will be introduced into codons encoding amino acids within a particular secondary structure at sites directed away from the interior of the polypeptide so that they will have the potential to interact with the OKL38-specific binding partner.

The phagemid-display method herein contemplates fusing a polynucleotide encoding the polypeptide or fragment (polynucleotide 1) to a second polynucleotide (polynucleotide 2) such that a fusion protein is generated during transcription. Polynucleotide 2 is typically a coat protein gene of a phage, and preferably it is the phage M13 gene III coat protein, or a fragment thereof. Fusion of polynucleotides 1 and 2 may be accomplished by inserting polynucleotide 2 into a particular site on a plasmid that contains polynucleotide 1, or by inserting polynucleotide 1 into a particular site on a plasmid that contains polynucleotide 2.

Between polynucleotide 1 and polynucleotide 2, DNA encoding a termination codon may be inserted, such termination codons being UAG (amber), UAA (ocher), and UGA (opel) (see for example, Davis et al., Microbiology (Harper and Row: New York, 1980), pages 237, 245-247, and 274). The termination codon expressed in a wild-type host cell results in the synthesis of the polynucleotide 1 protein product without the polynucleotide 2 protein attached. However, growth in a suppressor host cell results in the synthesis of detectable quantities of fused protein. Such suppressor host cells contain a tRNA modified to insert an amino acid in the termination codon position of the mRNA, thereby resulting in production of detectable amounts of the fusion protein. Suppressor host cells of this type are well known and described, such as E. coli suppressor strain (Bullock et al., 1987, BioTechniques, 5: 376-379). Any acceptable method may be used to place such a termination codon into the mRNA encoding the fusion polypeptide.

The suppressible codon may be inserted between the polynucleotide encoding the polypeptide or fragment and a second polynucleotide encoding at least a portion of a phage coat protein. Alternatively, the suppressible termination codon may be inserted adjacent to the fusion site by replacing the last amino acid triplet in the polypeptide/fragment or the first amino acid in the phage coat protein. When the phagemid containing the suppressible codon is grown in a suppressor host cell, it results in the detectable production of a fusion polypeptide containing the polypeptide or fragment and the coat protein. When the phagemid is grown in a non-suppressor host cell the polypeptide or fragment is synthesised substantially without fusion to the phage coat protein due to termination at the inserted suppressible triplet encoding UAG, UAA, or UGA. In the non-suppressor cell the polypeptide is synthesised and secreted from the host cell due to the absence of the fused phage coat protein which otherwise anchored it to the host cell.

The polypeptide or fragment may be altered at one or more selected codons. An alteration is defined as a substitution, deletion, or insertion of one or more codons in the gene encoding the polypeptide or fragment that results in a change in the amino acid sequence as compared with the unaltered or native sequence of the said polypeptide or fragment. Preferably, the alterations will be by substitution of at least one amino acid with any other amino acid in one or more regions of the molecule. The alterations may be produced by a variety of methods known in the art, as for example described in Section 2.3. These methods include, but are not limited to, oligonucleotide-mediated mutagenesis and cassette mutagenesis as described for example herein.

For preparing the OKL38-specific binding partner molecule and binding it with the phagemid, the binding partner molecule is attached to a suitable matrix such as agarose beads, acrylamide beads, glass beads, cellulose, various acrylic copolymers, hydroxyalkyl methacrylate gels, polyacrylic acid, polymethacrylic copolymers, nylon, neutral and ionic carriers, and the like. Attachment of the binding partner molecule to the matrix may be accomplished by methods described in i Methods Enzymol., 44: (1976), or by other means known in the art.

After attachment of the specific binding partner molecule to the matrix, the immobilized binding partner is contacted with the library of phagemid particles under conditions suitable for binding of at least a portion of the phagemid particles with the immobilized binding partner or target. Normally, the conditions, including pH, ionic strength, temperature, and the like will mimic physiological conditions.

Bound phagemid particles (“binders”) having high affinity for the immobilized target are separated from those having a low affinity (and thus do not bind to the target) by washing. Binders may be dissociated from the immobilized target by a variety of methods. These methods include competitive dissociation using the wild-type ligand, altering pH and/or ionic strength, and methods known in the art.

Suitable host cells are infected with the binders and helper phage, and the host cells are cultured under conditions suitable for amplification of the phagemid particles. The phagemid particles are then collected and the selection process is repeated one or more times until binders having the desired affinity for the target molecule are selected.

2.4.5 Rational Drug Design

Variants of an isolated polypeptide according to the invention or a biologically active fragment thereof may also be obtained using the principles of conventional or of rational drug design as for example described by Andrews, et al. (In: “PROCEEDINGS OF THE ALFRED BENZON SYMPOSIUM”, volume 28, pp. 145-165, Munksgaard, Copenhagen, 1990), McPherson, A. (1990, Eur. J. Biochem. 189: 1-24), Hol,. et al. (In: “MOLECULAR RECOGNITION: CHEMICAL AND BIOCHEMICAL PROBLEMS”, Roberts, S. M. (ed.); Royal Society of Chemistry; pp. 84-93, 1989), Hol, W. G. J. (1989, Arzneim-Forsch. 39: 1016-1018), Hol, W. G. J. (1986, Agnew Chem. Int. Ed. Engl. 25: 767-778).

In accordance with the methods of conventional drug design, the desired variant molecules are obtained by randomly testing molecules whose structures have an attribute in common with the structure of a parent polypeptide or biologically active fragment according to the invention. The quantitative contribution that results from a change in a particular group of a binding molecule can be determined by measuring the capacity of competition or cooperativity between the parent polypeptide or polypeptide fragment and the candidate polypeptide variant.

In one embodiment of rational drug design, the polypeptide variant is designed to share an attribute of the most stable three-dimensional conformation of a polypeptide or polypeptide fragment according to the invention. Thus, the variant may be designed to possess chemical groups that are oriented in a way sufficient to cause ionic, hydrophobic, or van der Waals interactions that are similar to those exhibited by the polypeptide or polypeptide fragment of the invention. In a second method of rational design, the capacity of a particular polypeptide or polypeptide fragment to undergo conformational “breathing” is exploited. Such “breathing”—the transient and reversible assumption of a different molecular conformation—is a well-appreciated phenomenon, and results from temperature, thermodynamic factors, and from the catalytic activity of the molecule. Knowledge of the 3-dimensional structure of the polypeptide or polypeptide fragment facilitates such an evaluation. An evaluation of the natural conformational changes of a polypeptide or polypeptide fragment facilitates the recognition of potential hinge sites, potential sites at which hydrogen bonding, ionic bonds or van der Waals bonds might form or might be eliminated due to the breathing of the molecule, etc. Such recognition permits the identification of the additional conformations that the polypeptide or polypeptide fragment could assume, and enables the rational design and production of mimetic polypeptide variants that share such conformations.

The preferred method for performing rational mimetic design employs a computer system capable of forming a representation of the three-dimensional structure of the polypeptide or polypeptide fragment (such as those obtained using RIBBON (Priestle, J., 1988, J. Mol. Graphics 21: 572), QUANTA (Polygen), InSite (Biosyn), or Nanovision (American Chemical Society)). Such analyses are exemplified by Hol, et al. (In: “MOLECULAR RECOGNITION: CHEMICAL AND BIOCHEMICAL PROBLEMS”, supra, Hol, W. G. J. (1989, supra) and Hol, W. G. J., (1986, supra).

In lieu of such direct comparative evaluations of candidate polypeptide variants, screening assays may be used to identify such molecules. Such assays will preferably exploit the capacity of the variant to bind to a OKL38-specific binding partner and/or to modulate an activity selected from the group consisting of cell differentiation, cell differentiation and tumorigenesis.

2.5 Polypeptide Derivatives

With reference to suitable derivatives of the invention, such derivatives include amino acid deletions and/or additions to a polypeptide, fragment or variant of the invention, wherein said derivatives bind a OKL38-specific binding partner or modulate at least one activity selected from the group consisting of cell differentiation, cell proliferation, and tumorigenesis. “Additions” of amino acids may include fusion of the polypeptides, fragments and polypeptide variants of the invention with other polypeptides or proteins. For example, it will be appreciated that said polypeptides, fragments or variants may be incorporated into larger polypeptides, and that such larger polypeptides may also be expected to bind an OKL38-specific binding partner or to modulate an activity as mentioned above.

The polypeptides, fragments or variants of the invention may be fused to a further protein, for example, which is not derived from the original host. The further protein may assist in the purification of the fusion protein. For instance, a polyhistidine tag or a maltose binding protein may be used in this respect as described in more detail below. Other possible fusion proteins are those which produce an immunomodulatory response. Particular examples of such proteins include Protein A or glutathione S-transferase (GST).

Other derivatives contemplated by the invention include, but are not limited to, modification to side chains, incorporation of unnatural amino acids and/or their derivatives during peptide, polypeptide or protein synthesis and the use of crosslinkers and other methods which impose conformational constraints on the polypeptides, fragments and variants of the invention.

Examples of side chain modifications contemplated by the present invention include modifications of amino groups such as by acylation with acetic anhydride; acylation of amino groups with succinic anhydride and tetrahydrophthalic anhydride; amidination with methylacetimidate; carbamoylation of amino groups with cyanate; pyridoxylation of lysine with pyridoxal-5-phosphate followed by reduction with NaBH₄; reductive alkylation by reaction with an aldehyde followed by reduction with NaBH₄; and trinitrobenzylation of amino groups with 2,4,6-trinitrobenzene sulfonic acid (TNBS).

The carboxyl group may be modified by carbodiimide activation via O-acylisourea formation followed by subsequent derivitization, by way of example, to a corresponding amide.

The guanidine group of arginine residues may be modified by formation of heterocyclic condensation products with reagents such as 2,3-butanedione, phenylglyoxal and glyoxal.

Sulfhydryl groups may be modified by methods such as performic acid oxidation to cysteic acid; formation of mercurial derivatives using 4-chloromercuriphenylsulphonic acid, 4-chloromercuribenzoate; 2-chloromercuri-4-nitrophenol, phenylmercury chloride, and other mercurials; formation of a mixed disulphides with other thiol compounds; reaction with maleimide, maleic anhydride or other substituted maleimide; carboxymethylation with iodoacetic acid or iodoacetamide; and carbamoylation with cyanate at alkaline pH.

Tryptophan residues may be modified, for example, by alkylation of the indole ring with 2-hydroxy-5-nitrobenzyl bromide or sulfonyl halides or by oxidation with N-bromosuccinimide.

Tyrosine residues may be modified by nitration with tetranitromethane to form a 3-nitrotyrosine derivative.

The imidazole ring of a histidine residue may be modified by N-carbethoxylation with diethylpyrocarbonate or by alkylation with iodoacetic acid derivatives.

Examples of incorporating unnatural amino acids and derivatives during peptide synthesis include but are not limited to, use of 4-amino butyric acid, 6-aminohexanoic acid, 4-amino-3-hydroxy-5-phenylpentanoic acid, 4-amino-3-hydroxy-6-methylheptanoic acid, t-butylglycine, norleucine, norvaline, phenylglycine, ornithine, sarcosine, 2-thienyl alanine and/or D-isomers of amino acids. A list of unnatural amino acids contemplated by the present invention is shown in TABLE C.

TABLE C

| Non-conventional amino acid | Non-conventional amino acid |
|---|---|
| α-aminobutyric acid | L-N-methylalanine |
| α-amino-α-methylbutyrate | L-N-methylarginine |
| aminocyclopropane-carboxylate | L-N-methylasparagine |
| aminoisobutyric acid | L-N-methylaspartic acid |
| aminonorbornyl-carboxylate | L-N-methylcysteine |
| cyclohexylalanine | L-N-methylglutamine |
| cyclopentylalanine | L-N-methylglutamic acid |
| L-N-methylisoleucine | L-N-methylhistidine |
| D-alanine | L-N-methylleucine |
| D-arginine | L-N-methyllysine |
| D-aspartic acid | L-N-methylmethionine |
| D-cysteine | L-N-methylnorleucine |
| D-glutamate | L-N-methylnorvaline |
| D-glutamic acid | L-N-methylornithine |
| D-histidine | L-N-methylphenylalanine |
| D-isoleucine | L-N-methylproline |
| D-leucine | L-N-medlylserine |
| D-lysine | L-N-methylthreonine |
| D-methionine | L-N-methyltryptophan |
| D-ornithine | L-N-methyltyrosine |
| D-phenylalanine | L-N-methylvaline |
| D-proline | L-N-methylethylglycine |
| D-serine | L-N-methyl-t-butylglycine |
| D-threonine | L-norleucine |
| D-tryptophan | L-norvaline |
| D-tyrosine | α-methyl-aminoisobutyrate |
| D-valine | α-methyl-γ-aminobutyrate |
| D-α-methylalanine | α-methylcyclohexylalanine |
| D-α-methylarginine | α-methylcylcopentylalanine |
| D-α-methylasparagine | α-methyl-α-napthylalanine |
| D-α-methylaspartate | α-methylpenicillamine |
| D-α-methylcysteine | N-(4-aminobutyl)glycine |
| D-α-methylglutamine | N-(2-aminoethyl)glycine |
| D-α-methylhistidine | N-(3-aminopropyl)glycine |
| D-α-methylisoleucine | N-amino-α-methylbutyrate |
| D-α-methylleucine | α-napthylalanine |
| D-α-methyllysine | N-benzylglycine |
| D-α-methylmethionine | N-(2-carbamylediyl)glycine |
| D-α-methylornithiine | N-(carbamylmethyl)glycine |
| D-α-methylphenylalanine | N-(2-carboxyethyl)glycine |
| D-α-methylproline | N-(carboxymethyl)glycine |
| D-α-methylserine | N-cyclobutylglycine |
| D-α-methylthreonine | N-cycloheptylglycine |
| D-α-methyltryptophan | N-cyclohexylglycine |
| D-α-methyltyrosine | N-cyclodecylglycine |
| L-α-methylleucine | L-α-methyllysine |
| L-α-methylmethionine | L-α-methylnorleucine |
| L-α-methylnorvatine | L-α-methylornithine |
| L-α-methylphenylalanine | L-α-methylproline |
| L-α-methylserine | L-α-methylthreonine |
| L-α-methyltryptophan | L-α-methyltyrosine |
| L-α-methylvaline | L-N-methylhomophenylalanine |
| N-(N-(2,2-diphenylethyl | N-(N-(3,3-diphenylpropyl |
| carbamylmethyl)glycine | carbamylmethyl)glycine |
| 1-carboxy-1-(2,2-diphenyl-ethyl | |
| amino)cyclopropane | |

Also contemplated is the use of crosslinkers, for example, to stabilise 3D conformations of the polypeptides, fragments or variants of the invention, using homo-bifunctional cross linkers such as bifunctional imido esters having (CH₂)ₙ spacer groups with n=1 to n=6, glutaraldehyde, N-hydroxysuccinimide esters and hetero-bifunctional reagents which usually contain an amino-reactive moiety such as N-hydroxysuccinimide and another group specific-reactive moiety such as maleimido or dithio moiety or carbodiimide. In addition, peptides can be conformationally constrained, for example, by introduction of double bonds between C_{α} and C_{β} atoms of amino acids, by incorporation of C_{α} and N_{α}-methylamino acids, and by formation of cyclic peptides or analogues by introducing covalent bonds such as forming an amide bond between the N and C termini between two side chains or between a side chain and the N or C terminus of the peptides or analogues. For example, reference may be made to: Marlowe (1993, Biorganic & Medicinal Chemistry Letters 3: 437-44) who describes peptide cyclization on TFA resin using trimethylsilyl (TMSE) ester as an orthogonal protecting group; Pallin and Tam (1995, J. Chem. Soc. Chem. Comm. 2021-2022) who describe the cyclization of unprotected peptides in aqueous solution by oxime formation; Algin et al (1994, Tetrahedron Letters 35: 9633-9636) who disclose solid-phase synthesis of head-to-tail cyclic peptides via lysine side-chain anchoring; Kates et al (1993, Tetrahedron Letters 34: 1549-1552) who describe the production of head-to-tail cyclic peptides by three-dimensional solid phase strategy; Tumelty et al (1994, J. Chem. Soc. Chem. Comm. 1067-1068) who describe the synthesis of cyclic peptides from an immobilized activated intermediate, wherein activation of the immobilized peptide is carried out with N-protecting group intact and subsequent removal leading to cyclization; McMurray et al (1994, Peptide Research 7: 195-206) who disclose head-to-tail cyclization of peptides attached to insoluble supports by means of the side chains of aspartic and glutamic acid; Hruby et al (1994, Reactive Polymers 22: 231-241) who teach an alternate method for cyclizing peptides via solid supports; and Schmidt and Langer (1997, J. Peptide Res. 49: 67-73) who disclose a method for synthesising cyclotetrapeptides and cyclopentapeptides. The foregoing methods may be used to produce conformationally constrained polypeptides that bind to an OKL38-specific binding partner or to modulate an activity selected from the group consisting of cell differentiation, cell proliferation and tumorigenesis.

The invention also contemplates polypeptides, fragments or variants of the invention that have been modified using ordinary molecular biological techniques so as to improve their resistance to proteolytic degradation or to optimise solubility properties or to render them more suitable as an immunogenic agent.

2.6 Methods of Preparing the Polypeptides of the Invention

Polypeptides of the inventions may be prepared by any suitable procedure known to those of skill in the art. For example, the polypeptides may be prepared by a procedure including the steps of; preparing a recombinant polynucleotide comprising a nucleotide sequence encoding a polypeptide comprising the sequence set forth in any one of SEQ ID NO: 2, or variant or derivative of these, which nucleotide sequence is operably linked to a regulatory polynucleotide; introducing the recombinant polynucleotide into a suitable host cell; culturing the host cell to express recombinant polypeptide from said recombinant polynucleotide; and isolating the recombinant polypeptide.

Suitably, said nucleotide sequence comprises the sequence set forth in any one of SEQ ID NO: 1, 3 and 5.

The recombinant polynucleotide preferably comprises either an expression vector that may be a self-replicating extra-chromosomal vector such as a plasmid, or a vector that integrates into a host genome.

The transcriptional and translational regulatory nucleic acid will generally be appropriate for the host cell used for expression. Numerous types of appropriate expression vectors and suitable regulatory sequences are known in the art for a variety of host cells. Typically, the transcriptional and translational regulatory nucleic acid may include, but is not limited to, promoter sequences, leader or signal sequences, ribosomal binding sites, transcriptional start and stop sequences, translational start and termination sequences, and enhancer or activator sequences. Constitutive or inducible promoters as known in the art are contemplated by the invention. The promoters may be either naturally occurring promoters, or hybrid promoters that combine elements of more than one promoter.

In a preferred embodiment, the expression vector contains a selectable marker gene to allow the selection of transformed host cells. Selection genes are well known in the art and will vary with the host cell used.

The expression vector may also include a fusion partner (typically provided by the expression vector) so that the recombinant polypeptide of the invention is expressed as a fusion polypeptide with said fusion partner. The main advantage of fusion partners is that they assist identification and/or purification of said fusion polypeptide. In order to express said fusion polypeptide, it is necessary to ligate a polynucleotide according to the invention into the expression vector so that the translational reading frames of the fusion partner and the polynucleotide coincide. Well known examples of fusion partners include, but are not limited to, glutathione-S-transferase (GST), Fc potion of human IgG, maltose binding protein (MBP) and hexahistidine (HIS₆), which are particularly useful for isolation of the fusion polypeptide by affinity chromatography. For the purposes of fusion polypeptide purification by affinity chromatography, relevant matrices for affinity chromatography are glutathione-, amylose-, and nickel- or cobalt-conjugated resins respectively. Many such matrices are available in “kit” form, such as the QIAexpress™ system (Qiagen) useful with (HIS₆) fusion partners and the Pharmacia GST purification system. In a preferred embodiment, the recombinant polynucleotide is expressed in the commercial vector pFLAG as described more fully hereinafter.

Another fusion partner well known in the art is green fluorescent protein (GFP). This fusion partner serves as a fluorescent “tag” which allows the fusion polypeptide of the invention to be identified by fluorescence microscopy or by flow cytometry. The GFP tag is useful when assessing subcellular localisation of the fusion polypeptide of the invention, or for isolating cells which express the fusion polypeptide of the invention. Flow cytometric methods such as fluorescence activated cell sorting (FACS) are particularly useful in this latter application. Preferably, the fusion partners also have protease cleavage sites, such as for Factor Xₐ or Thrombin, which allow the relevant protease to partially digest the fusion polypeptide of the invention and thereby liberate the recombinant polypeptide of the invention therefrom. The liberated polypeptide can then be isolated from the fusion partner by subsequent chromatographic separation. Fusion partners according to the invention also include within their scope “epitope tags”, which are usually short peptide sequences for which a specific antibody is available. Well known examples of epitope tags for which specific monoclonal antibodies are readily available include c-Myc, influenza virus, haemagglutinin and FLAG tags.

The step of introducing into the host cell the recombinant polynucleotide may be effected by any suitable method including transfection, and transformation, the choice of which will be dependent on the host cell employed. Such methods are well known to those of skill in the art. Recombinant polypeptides of the invention may be produced by culturing a host cell transformed with an expression vector containing nucleic acid encoding a polypeptide, biologically active fragment, variant or derivative according to the invention. The conditions appropriate for protein expression will vary with the choice of expression vector and the host cell. This is easily ascertained by one skilled in the art through routine experimentation.

Suitable host cells for expression may be prokaryotic or eukaryotic. One preferred host cell for expression of a polypeptide according to the invention is a bacterium. The bacterium used may be Escherichia coli. Alternatively, the host cell may be an insect cell such as, for example, SF9 cells that may be utilised with a baculovirus expression system.

The recombinant protein may be conveniently prepared by a person skilled in the art using standard protocols as for example described in Sambrook, et al., MOLECULAR CLONING. A LABORATORY MANUAL (Cold Spring Harbor Press, 1989), in particular Sections 16 and 17; Ausubel et al., CURRENT PROTOCOLS IN MOLECULAR BIOLOGY (John Wiley & Sons, Inc. 1994-1998), in particular Chapters 10 and 16; and Coligan et al., CURRENT PROTOCOLS IN PROTEIN SCIENCE (John Wiley & Sons, Inc. 1995-1997), in particular Chapters 1, 5 and 6.

Alternatively, the polypeptide, fragments, variants or derivatives of the invention may be synthesised using solution synthesis or solid phase synthesis as described, for example, in Chapter 9 of Atherton and Shephard (supra) and in Roberge et al (1995, Science 269: 202).

3. Polynucleotides of the Invention

3.1 Polynucleotides Encoding Polypeptides of the Invention

The invention further provides a polynucleotide that encodes a polypeptide, fragment, variant or derivative as defined above. In one embodiment, the polynucleotide comprises the entire sequence of nucleotides set forth in SEQ ID NO: 1. SEQ ID NO: 1 corresponds to the full-length human 1607 bp OKL38 cDNA sequence. This sequence defines: (1) a 5′ untranslated region from nucleotide 1 through nucleotide 126; (2) an open reading frame from nucleotide 127 through nucleotide 1078; (3) a 3′ untranslated region from nucleotide 1079 through nucleotide 1607; (4) a polyadenylation signal from nucleotide 1576 through 1582; and (5) a polyadenylation tail from nucleotide 1598. Suitably, the polynucleotide comprises the sequence set forth in SEQ ID NO: 3, which defines the entire open reading frame (ORF) of OKL38.

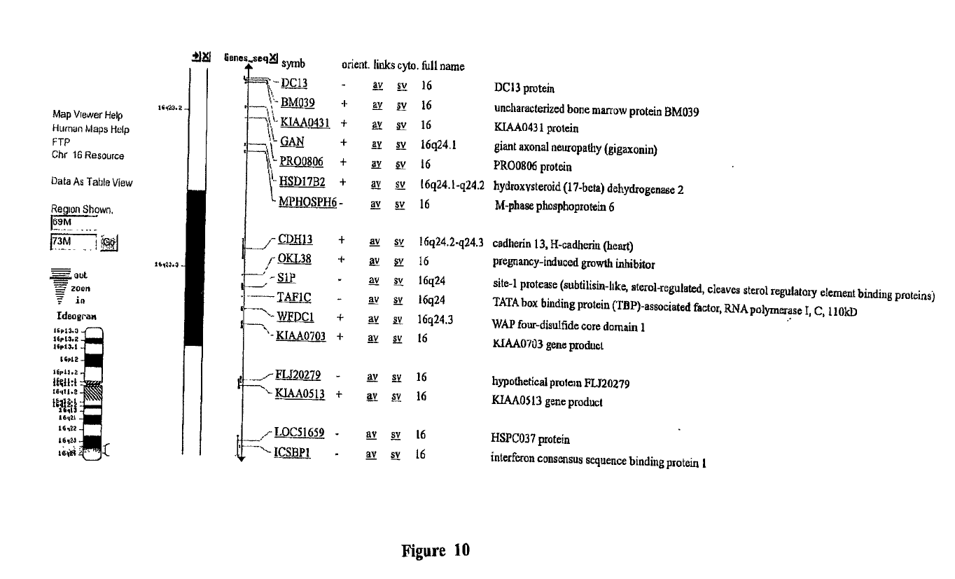

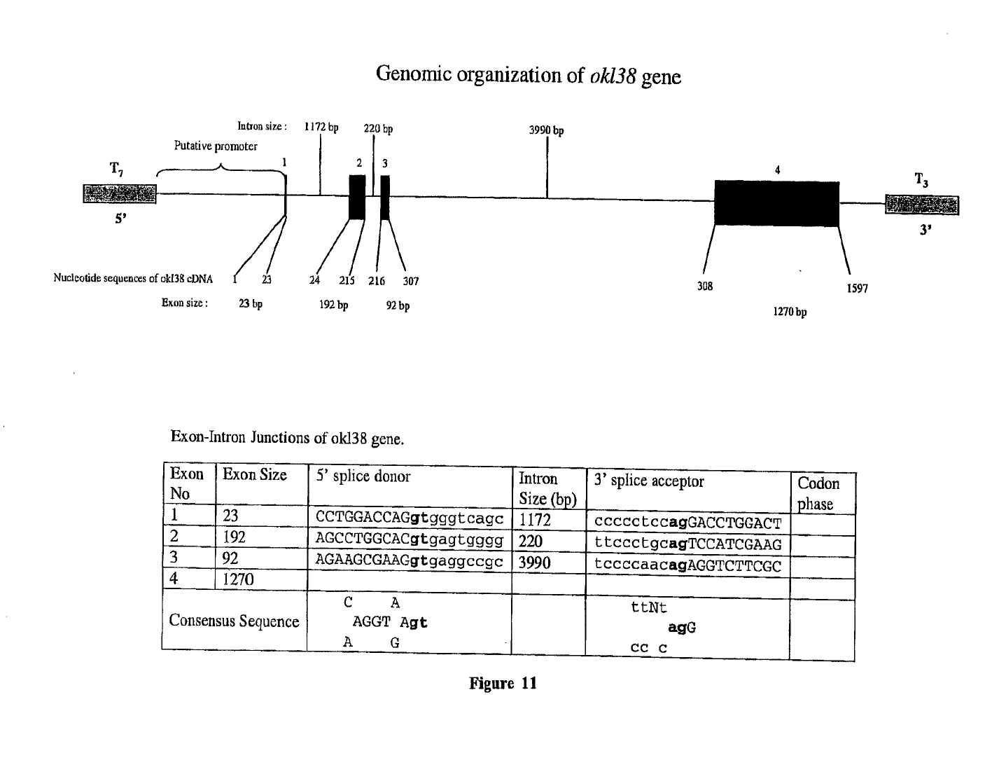

The invention also provides an isolated polynucleotide comprising an OKL38 gene or fragment thereof. Suitably, the polynucleotide comprises the entire sequence of nucleotides set forth in SEQ ID NO: 5. SEQ ID NO: 5 corresponds to a 12235 bp human genomic sequence for OKL38. This sequence defines: (1) a first exon, comprising a 5′ untranslated region, from nucleotide 5808 through nucleotide 5830; (2) a first intron from nucleotide 5831 through nucleotide 7002; (3) a second exon from nucleotide 7003 through nucleotide 7194, comprising a 5′ portion of the OKL38 ORF from nucleotide 7105 through nucleotide 7194; (4) a second intron from nucleotide 7195 through nucleotide 7414; (5) a third exon, comprising a portion of the OKL38 ORF, from nucleotide 7415 through nucleotide 7506; (6) a third intron from nucleotide 7507 through nucleotide 11496; and (7) a fourth exon, comprising a 3′ portion of the OKL38 ORF, from nucleotide 11497 through nucleotide 12235. A chromosomal map showing the location of OKL38, together with a schematic showing the genomic organisation of OKL38, are shown in FIGS. 10 and 11, respectively.

The OKL38 gene and portions thereof, including exons and introns, have utility in a variety of applications, including its use in identifying aberrant OKL38 genes and transcripts that associate with enhancement or promotion of cell proliferation and/or tumorigenesis. The OKL38 gene and portions thereof and flanking polynucleotide sequences also have utility for isolating or otherwise produce polynucleotide sequences, including genomic and cDNA sequences of other animals, which could be taken advantage to produce non-human transgenic animals. Useful sequences for producing transgenic animals include, but are not restricted to, open reading frames encoding specific polypeptides or domains, introns, and adjacent 5′ and 3′ non-coding nucleotide sequences involved in the regulation of expression, up to about 1 kb beyond the coding region, but possibly further in either direction.

3.2 Polynucleotides Variants

In general, polynucleotide variants according to the invention comprise regions that show at least 50%, more suitably at least 70%, preferably at least 80%, more preferably at least 90%, even more preferably at least 95% and still more preferably at least 95% sequence identity over a reference polynucleotide sequence of identical size (“comparison window”) or when compared to an aligned sequence in which the alignment is performed by a computer homology program known in the art. What constitutes suitable variants may be determined by conventional techniques. For example, a polynucleotide according to SEQ ID NO: 1, 3 and 5 can be mutated using random mutagenesis (e.g., transposon mutagenesis), oligonucleotide-mediated (or site-directed) mutagenesis, PCR mutagenesis and cassette mutagenesis of an earlier prepared variant or non-variant version of an isolated natural promoter according to the invention.

Oligonucleotide-mediated mutagenesis is a preferred method for preparing nucleotide substitution variants of a polynucleotide of the invention. This technique is well known in the art as, for example, described by Adelman et al. (1983, DNA 2:183). Briefly, a polynucleotide according to any one of SEQ ID NO: 1 or 3 is altered by hybridising an oligonucleotide encoding the desired mutation to a template DNA, wherein the template is the single-stranded form of a plasmid or bacteriophage containing the unaltered or parent DNA sequence. After hybridisation, a DNA polymerase is used to synthesise an entire second complementary strand of the template that will thus incorporate the oligonucleotide primer, and will code for the selected alteration in said parent DNA sequence.

Generally, oligonucleotides of at least 25 nucleotides in length are used. An optimal oligonucleotide will have 12 to 15 nucleotides that are completely complementary to the template on either side of the nucleotide(s) coding for the mutation. This ensures that the oligonucleotide will hybridise properly to the single-stranded DNA template molecule.

The DNA template can be generated by those vectors that are either derived from bacteriophage M 13 vectors, or those vectors that contain a single-stranded phage origin of replication as described by Viera et al. (1987, Methods Enzymol. 153:3). Thus, the DNA that is to be mutated may be inserted into one of the vectors to generate single-stranded template. Production of single-stranded template is described, for example, in Sections 4.21-4.41 of Sambrook et al. (1989, supra).

Alternatively, the single-stranded template may be generated by denaturing double-stranded plasmid (or other DNA) using standard techniques.

For alteration of the native DNA sequence, the oligonucleotide is hybridised to the single-stranded template under suitable hybridisation conditions. A DNA polymerising enzyme, usually the Klenow fragment of DNA polymerase I, is then added to synthesise the complementary strand of the template using the oligonucleotide as a primer for synthesis. A heteroduplex molecule is thus formed such that one strand of DNA encodes the mutated form of the polypeptide or fragment under test, and the other strand (the original template) encodes the native unaltered sequence of the polypeptide or fragment under test. This heteroduplex molecule is then transformed into a suitable host cell, usually a prokaryote such as E. coli. After the cells are grown, they are plated onto agarose plates and screened using the oligonucleotide primer having a detectable label to identify the bacterial colonies having the mutated DNA. The resultant mutated DNA fragments are then cloned into suitable expression hosts such as E. coli using conventional technology and clones that retain the desired antigenic activity are detected. Where the clones have been derived using random mutagenesis techniques, positive clones would have to be sequenced in order to detect the mutation.

Alternatively, linker-scanning mutagenesis of DNA may be used to introduce clusters of point mutations throughout a sequence of interest that has been cloned into a plasmid vector. For example, reference may be made to Ausubel et al., supra, (in particular, Chapter 8.4) which describes a first protocol that uses complementary oligonucleotides and requires a unique restriction site adjacent to the region that is to be mutagenized. A nested series of deletion mutations is first generated in the region. A pair of complementary oligonucleotides is synthesised to fill in the gap in the sequence of interest between the linker at the deletion endpoint and the nearby restriction site. The linker sequence actually provides the desired clusters of point mutations as it is moved or “scanned” across the region by its position at the varied endpoints of the deletion mutation series. An alternate protocol is also described by Ausubel et al., supra, which makes use of site directed mutagenesis procedures to introduce small clusters of point mutations throughout the target region. Briefly, mutations are introduced into a sequence by annealing a synthetic oligonucleotide containing one or more mismatches to the sequence of interest cloned into a single-stranded M13 vector. This template is grown in an E. coli dut⁻ ung⁻ strain, which allows the incorporation of uracil into the template strand. The oligonucleotide is annealed to the template and extended with T4 DNA polymerase to create a double-stranded heteroduplex. Finally, the heteroduplex is introduced into a wild-type E. coli strain, which will prevent replication of the template strand due to the presence of apurinic sites (generated where uracil is incorporated), thereby resulting in plaques containing only mutated DNA.

Region-specific mutagenesis and directed mutagenesis using PCR may also be employed to construct polynucleotide variants according to the invention. In this regard, reference may be made, for example, to Ausubel et al., supra, in particular Chapters 8.2A and 8.5.

Alternatively, suitable polynucleotide sequence variants of the invention may be prepared according to the following procedure:

creating primers which are optionally degenerate wherein each comprises a portion of a reference polynucleotide encoding a reference polypeptide or fragment of the invention, preferably encoding the sequence set forth in SEQ ID NO: 2, more preferably comprising the sequence set forth in any one of SEQ ID NO: 1, 3 and 5;

obtaining a nucleic acid extract from an organism, which is preferably an animal, and more preferably a mammal; and

using said primers to amplify, via nucleic acid amplification techniques, at least one amplification product from said nucleic acid extract, wherein said amplification product corresponds to a polynucleotide variant.

Suitable nucleic acid amplification techniques are well known to the skilled addressee, and include polymerase chain reaction (PCR) as for example described in Ausubel et al. (supra); strand displacement amplification (SDA) as for example described in U.S. Pat. No 5,422,252; rolling circle replication (RCR) as for example described in Liu et al., (1996, J. Am. Chem. Soc. 118:1587-1594 and International application WO 92/01813) and Lizardi et al., (International Application WO 97/19193); nucleic acid sequence-based amplification (NASBA) as for example described by Sooknanan et al., (1994, Biotechniques 17:1077-1080); and Q-β replicase amplification as for example described by Tyagi et al., (1996, Proc. Natl. Acad. Sci. USA 93: 5395-5400).

Typically, polynucleotide variants that are substantially complementary to a reference polynucleotide are identified by blotting techniques that include a step whereby nucleic acids are immobilised on a matrix (preferably a synthetic membrane such as nitrocellulose), followed by a hybridisation step, and a detection step. Southern blotting is used to identify a complementary DNA sequence; northern blotting is used to identify a complementary RNA sequence. Dot blotting and slot blotting can be used to identify complementary DNA/DNA, DNA/RNA or RNA/RNA polynucleotide sequences. Such techniques are well known by those skilled in the art, and have been described in Ausubel et al (1994-1998, supra) at pages 2.9.1 through 2.9.20.

According to such methods, Southern blotting involves separating DNA molecules according to size by gel electrophoresis, transferring the size-separated DNA to a synthetic membrane, and hybridising the membrane-bound DNA to a complementary nucleotide sequence labelled radioactively, enzymatically or fluorochromatically. In dot blotting and slot blotting, DNA samples are directly applied to a synthetic membrane prior to hybridisation as above.

An alternative blotting step is used when identifying complementary polynucleotides in a cDNA or genomic DNA library, such as through the process of plaque or colony hybridisation. A typical example of this procedure is described in Sambrook et al. (“Molecular Cloning. A Laboratory Manual”, Cold Spring Harbour Press, 1989) Chapters 8-12.

Typically, the following general procedure can be used to determine hybridisation conditions. Polynucleotides are blotted/transferred to a synthetic membrane, as described above. A reference polynucleotide such as a polynucleotide of the invention is labelled as described above, and the ability of this labelled polynucleotide to hybridise with an immobilised polynucleotide is analysed.

A skilled addressee will recognise that a number of factors influence hybridisation. The specific activity of radioactively labelled polynucleotide sequence should typically be greater than or equal to about 10⁸ dpm/mg to provide a detectable signal. A radiolabelled nucleotide sequence of specific activity 10⁸ to 10⁹ dpm/mg can detect approximately 0.5 pg of DNA. It is well known in the art that sufficient DNA must be immobilised on the membrane to permit detection. It is desirable to have excess immobilised DNA, usually 10 μg. Adding an inert polymer such as 10% (w/v) dextran sulfate (MW 500,000) or polyethylene glycol 6000 during hybridisation can also increase the sensitivity of hybridisation (see Ausubel supra at 2.10.10).

To achieve meaningful results from hybridisation between a polynucleotide immobilised on a membrane and a labelled polynucleotide, a sufficient amount of the labelled polynucleotide must be hybridised to the immobilised polynucleotide following washing. Washing ensures that the labelled polynucleotide is hybridised only to the immobilised polynucleotide with a desired degree of complementarity to the labelled polynucleotide.

It will be understood that polynucleotide variants according to the invention will hybridise to a reference polynucleotide under at least low stringency conditions. Reference herein to low stringency conditions include and encompass from at least about 1% v/v to at least about 15% v/v formamide and from at least about 1 M to at least about 2 M salt for hybridisation at 42° C., and at least about 1 M to at least about 2 M salt for washing at 42° C. Low stringency conditions also may include 1% Bovine Serum Albumin (BSA), 1 mM EDTA, 0.5 M NaHPO₄ (pH 7.2), 7% SDS for hybridisation at 65° C., and (i) 2×SSC, 0.1% SDS; or (ii) 0.5% BSA, 1 mM EDTA, 40 mM NaHPO₄ (pH 7.2), 5% SDS for washing at room temperature.

Suitably, the polynucleotide variants hybridise to a reference polynucleotide under at least medium stringency conditions. Medium stringency conditions include and encompass from at least about 16% v/v to at least about 30% v/v formamide and from at least about 0.5 M to at least about 0.9 M salt for hybridisation at 42° C., and at least about 0.1 M to at least about 0.2 M salt for washing at 55° C. Medium stringency conditions also may include 1% Bovine Serum Albumin (BSA), 1 mM EDTA, 0.5 M NaHPO₄ (pH 7.2), 7% SDS for hybridisation at 65° C., and (i) 2×SSC, 0.1% SDS; or (ii) 0.5% BSA, 1 mM EDTA, 40 mM NaHPO₄ (pH 7.2), 5% SDS for washing at 60-65° C.

Preferably, the polynucleotide variants hybridise to a reference polynucleotide under high stringency conditions. High stringency conditions include and encompass from at least about 31% v/v to at least about 50% v/v formamide and from about 0.01 M to about 0.15 M salt for hybridisation at 42° C., and about 0.01 M to about 0.02 M salt for washing at 55° C. High stringency conditions also may include 1% BSA, 1 mM EDTA, 0.5 M NaHPO₄ (pH 7.2), 7% SDS for hybridisation at 65° C., and (i) 0.2×SSC, 0.1% SDS; or (ii) 0.5% BSA, 1 mM EDTA, 40 mM NaHPO₄ (pH 7.2), 1% SDS for washing at a temperature in excess of 65° C.

Other stringent conditions are well known in the art. A skilled addressee will recognise that various factors can be manipulated to optimise the specificity of the hybridisation. Optimisation of the stringency of the final washes can serve to ensure a high degree of hybridisation. For detailed examples, see Ausubel et al., supra at pages 2.10.1 to 2.10.16 and Sambrook et al. (1989, supra) at sections 1.101 to 1.104.

While stringent washes are typically carried out at temperatures from about 42° C. to 68° C., one skilled in the art will appreciate that other temperatures may be suitable for stringent conditions. Maximum hybridisation rate typically occurs at about 20° C. to 25° C. below the Tₘ for formation of a DNA-DNA hybrid. It is well known in the art that the Tₘ is the melting temperature, or temperature at which two complementary polynucleotide sequences dissociate. Methods for estimating Tₘ are well known in the art (see Ausubel et al., supra at page 2.10.8).

In general, the Tₘ of a perfectly matched duplex of DNA may be predicted as an approximation by the formula:

Tₘ=81.5+16.6(log₁₀ M)+0.41(%G+C)−0.63(% formamide)−(600/length)

wherein: M is the concentration of Na⁺, preferably in the range of 0.01 molar to 0.4 molar; %G+C is the sum of guanosine and cytosine bases as a percentage of the total number of bases, within the range between 30% and 75% G+C; % formamide is the percent formamide concentration by volume; length is the number of base pairs in the DNA duplex.

The Tₘ of a duplex DNA decreases by approximately 1° C. with every increase of 1% in the number of randomly mismatched base pairs. Washing is generally carried out at Tₘ−15° C. for high stringency, or Tₘ−30° C. for moderate stringency.

In a preferred hybridisation procedure, a membrane (e.g., a nitrocellulose membrane or a nylon membrane) containing immobilised DNA is hybridised overnight at 42° C. in a hybridisation buffer (50% deionised formamide, 5×SSC, 5×Denhardt's solution (0.1% ficoll, 0.1% polyvinylpyrollidone and 0.1% bovine serum albumin), 0.1% SDS and 200 mg/mL denatured salmon sperm DNA) containing labelled probe. The membrane is then subjected to two sequential medium stringency washes (i.e., 2×SSC, 0.1% SDS for 15 min at 45° C., followed by 2×SSC, 0.1% SDS for 15 min at 50° C.), followed by two sequential higher stringency washes (i.e., 0.2×SSC, 0.1% SDS for 12 min at 55° C. followed by 0.2×SSC and 0.1%SDS solution for 12 min at 65-68° C.

Methods for detecting a labelled polynucleotide hybridised to an immobilised polynucleotide are well known to practitioners in the art. Such methods include autoradiography, phosphorimaging, and chemiluminescent, fluorescent and colorimetric detection.

4. Antigen-Binding Molecules

The invention also contemplates antigen-binding molecules that bind specifically to the aforementioned polypeptides, fragments, variants and derivatives. For example, the antigen-binding molecules may comprise whole polyclonal antibodies. Such antibodies may be prepared, for example, by injecting a polypeptide, fragment, variant or derivative of the invention into a production species, which may include mice or rabbits, to obtain polyclonal antisera. Methods of producing polyclonal antibodies are well known to those skilled in the art. Exemplary protocols which may be used are described for example in Coligan et al., CURRENT PROTOCOLS IN IMMUNOLOGY, (John Wiley & Sons, Inc, 1991), and Ausubel et al., (1994-1998, supra), in particular Section III of Chapter 11.

In lieu of the polyclonal antisera obtained in the production species, monoclonal antibodies may be produced using the standard method as described, for example, by Kohler and Milstein (1975, Nature 256, 495-497), or by more recent modifications thereof as described, for example, in Coligan et al., (1991, supra) by immortalising spleen or other antibody producing cells derived from a production species which has been inoculated with one or more of the polypeptides, fragments, variants or derivatives of the invention.

The invention also contemplates as antigen-binding molecules Fv, Fab, Fab′ and F(ab′)₂ immunoglobulin fragments.

Alternatively, the antigen-binding molecule may comprise a synthetic stabilised Fv fragment. Exemplary fragments of this type include single chain Fv fragments (sFv, frequently termed scFv) in which a peptide linker is used to bridge the N terminus or C terminus of a V_{H} domain with the C terminus or N-terminus, respectively, of a V_{L} domain. ScFv lack all constant parts of whole antibodies and are not able to activate complement. Suitable peptide linkers for joining the V_{H} and V_{L} domains are those which allow the V_{H} and V_{L} domains to fold into a single polypeptide chain having an antigen binding site with a three dimensional structure similar to that of the antigen binding site of a whole antibody from which the Fv fragment is derived. Linkers having the desired properties may be obtained by the method disclosed in U.S. Pat. No. 4,946,778. However, in some cases a linker is absent. ScFvs may be prepared, for example, in accordance with methods outlined in Kreber et al (Krebber et al. 1997, J. Immunol. Methods; 201(1): 35-55). Alternatively, they may be prepared by methods described in U.S. Pat. No. 5,091,513, European Patent No 239,400 or the articles by Winter and Milstein (1991, Nature 349:293) and Plückthun et al (1996, In Antibody engineering: A practical approach. 203-252).

Alternatively, the synthetic stabilised Fv fragment comprises a disulphide stabilised Fv (dsFv) in which cysteine residues are introduced into the V_{H} and V_{L} domains such that in the fully folded Fv molecule the two residues will form a disulphide bond therebetween. Suitable methods of producing dsFv are described for example in (Glockscuther et al. Biochem. 29: 1363-1367; Reiter et al. 1994, J. Biol. Chem. 269: 18327-18331; Reiter et al. 1994, Biochem. 33: 5451-5459; Reiter et al. 1994. Cancer Res. 54: 2714-2718; Webber et al. 1995, Mol. Immunol. 32: 249-258).

Also contemplated as antigen-binding molecules are single variable region domains (termed dAbs) as for example disclosed in (Ward et al. 1989, Nature 341: 544-546; Hamers-Casterman et al. 1993, Nature. 363: 446-448; Davies & Riechmann, 1994, FEBS Lett. 339: 285-290).

Alternatively, the antigen-binding molecule may comprise a “minibody”. In this regard, minibodies are small versions of whole antibodies, which encode in a single chain the essential elements of a whole antibody. Suitably, the minibody is comprised of the V_{H} and V_{L} domains of a native antibody fused to the hinge region and CH3 domain of the immunoglobulin molecule as, for example, disclosed in U.S. Pat. No. 5,837,821.

In an alternate embodiment, the antigen binding molecule may comprise non-immunoglobulin derived, protein frameworks. For example, reference may be made to (Ku & Schultz, 1995, Proc. Natl. Acad. Sci. USA, 92: 652-6556) which discloses a four-helix bundle protein cytochrome b562 having two loops randomised to create complementarity determining regions (CDRs), which have been selected for antigen binding.

The antigen-binding molecule may be multivalent (i.e., having more than one antigen binding site). Such multivalent molecules may be specific for one or more antigens. Multivalent molecules of this type may be prepared by dimerization of two antibody fragments through a cysteinyl-containing peptide as, for example disclosed by (Adams et al., 1993, Cancer Res. 53: 4026-4034; Cumber et al., 1992, J. Immunol. 149: 120-126). Alternatively, dimerization may be facilitated by fusion of the antibody fragments to amphiphilic helices that naturally dimerize (Pack P. Plünckthun, 1992, Biochem. 31: 1579-1584), or by use of domains (such as the leucine zippers jun and fos) that preferentially heterodimerize (Kostelny et al., 1992, J. Immunol. 148: 1547-1553). In an alternate embodiment, the multivalent molecule may comprise a multivalent single chain antibody (multi-scFv) comprising at least two scFvs linked together by a peptide linker. In this regard, non-covalently or covalently linked scFv dimers termed “diabodies” may be used. Multi-scFvs may be bispecific or greater depending on the number of scFvs employed having different antigen binding specificities. Multi-scFvs may be prepared for example by methods disclosed in U.S. Pat. No. 5,892,020.

The antigen-binding molecules of the invention may be used for affinity chromatography in isolating a natural or recombinant polypeptide or biologically active fragment of the invention. For example reference may be made to immunoaffinity chromatographic procedures described in Chapter 9.5 of Coligan et al., (1995-1997, supra).

The antigen-binding molecules can be used to screen expression libraries for variant polypeptides of the invention as described herein. They can also be used to detect polypeptides, fragments, variants and derivatives of the invention as described hereinafter.

5. Methods of Detection

5.1 Detection of Polypeptides According to the Invention

The invention also extends to a method of detecting in a sample a polypeptide, fragment, variant or derivative as broadly described above, comprising contacting the sample with an antigen-binding molecule as described in Section 4 and detecting the presence of a complex comprising the said antigen-binding molecule and the said polypeptide, fragment, variant or derivative in said contacted sample.

Any suitable technique for determining formation of the complex may be used. For example, an antigen-binding molecule according to the invention, having a reporter molecule associated therewith may be utilised in immunoassays. Such immunoassays include, but are not limited to, radioimmunoassays (RIAs), enzyme-linked immunosorbent assays (ELISAs) and immunochromatographic techniques (ICTs), Western blotting which are well known those of skill in the art. For example, reference may be made to “CURRENT PROTOCOLS IN IMMUNOLOGY” (1994, supra) which discloses a variety of immunoassays that may be used in accordance with the present invention. Immunoassays may include competitive assays as understood in the art or as for example described infra. It will be understood that the present invention encompasses qualitative and quantitative immunoassays.

Suitable immunoassay techniques are described for example in U.S. Pat. Nos. 4,016,043, 4,424,279 and 4,018,653. These include both single-site and two-site assays of the non-competitive types, as well as the traditional competitive binding assays. These assays also include direct binding of a labelled antigen-binding molecule to a target antigen.

Two site assays are particularly favoured for use in the present invention. A number of variations of these assays exist, all of which are intended to be encompassed by the present invention. Briefly, in a typical forward assay, an unlabelled antigen-binding molecule such as an unlabelled antibody is immobilized on a solid substrate and the sample to be tested brought into contact with the bound molecule. After a suitable period of incubation, for a period of time sufficient to allow formation of an antibody-antigen complex, another antigen-binding molecule, suitably a second antibody specific to the antigen, labelled with a reporter molecule capable of producing a detectable signal is then added and incubated, allowing time sufficient for the formation of another complex of antibody-antigen-labelled antibody. Any unreacted material is washed away and the presence of the antigen is determined by observation of a signal produced by the reporter molecule. The results may be either qualitative, by simple observation of the visible signal, or may be quantitated by comparing with a control sample containing known amounts of antigen. Variations on the forward assay include a simultaneous assay, in which both sample and labelled antibody are added simultaneously to the bound antibody. These techniques are well known to those skilled in the art, including minor variations as will be readily apparent. In accordance with the present invention, the sample may be one that might contain an antigen including serum, whole blood, and plasma or lymph fluid. Preferably, the sample is a tissue biopsy. The tissue biopsy suitable comprises tissue from an organ selected from any one or more of breast, liver, kidney, testis, bladder and lung.

In the typical forward assay, a first antibody having specificity for the antigen or antigenic parts thereof is either covalently or passively bound to a solid surface. The solid surface is typically glass or a polymer, the most commonly used polymers being cellulose, polyacrylamide, nylon, polystyrene, polyvinyl chloride or polypropylene. The solid supports may be in the form of tubes, beads, discs of microplates, or any other surface suitable for conducting an immunoassay. The binding processes are well known in the art and generally consist of cross-linking covalently binding or physically adsorbing, the polymer-antibody complex is washed in preparation for the test sample. An aliquot of the sample to be tested is then added to the solid phase complex and incubated for a period of time sufficient and under suitable conditions to allow binding of any antigen present to the antibody. Following the incubation period, the antigen-antibody complex is washed and dried and incubated with a second antibody specific for a portion of the antigen. The second antibody has generally a reporter molecule associated therewith that is used to indicate the binding of the second antibody to the antigen. The amount of labelled antibody that binds, as determined by the associated reporter molecule, is proportional to the amount of antigen bound to the immobilised first antibody.

An alternative method involves immobilising the antigen in the biological sample and then exposing the immobilized antigen to specific antibody that may or may not be labelled with a reporter molecule. Depending on the amount of target and the strength of the reporter molecule signal, a bound antigen may be detectable by direct labelling with the antibody. Alternatively, a second labelled antibody, specific to the first antibody is exposed to the target-first antibody complex to form a target-first antibody-second antibody tertiary complex. The complex is detected by the signal emitted by the reporter molecule.

Alternatively, the polypeptides, variants and derivatives as broadly described above may be detected, quantified or semi-quantified by flow cytometric analysis and immunofluorescent staining of intracellular antigens with antibody to quantify and compare individual cells for antigen expression levels as for example disclosed by Clevenger, et al (1987, J Cell Physiol. 130: 336-343) and by Kuhar and Lehman (1991, Oncogene 6: 1499-1506).

In an alternate embodiment, detection, quantification and semi-quantification may be effected using immunohistological analysis (e.g., diaminobenzidine staining of thin sections) as is known in the art.

From the foregoing, it will be appreciated that the reporter molecule associated with the antigen-binding molecule may include the following:

(a) direct attachment of the reporter molecule to the antigen-binding molecule;

(b) indirect attachment of the reporter molecule to the antigen-binding molecule; i.e., attachment of the reporter molecule to another assay reagent which subsequently binds to the antigen-binding molecule; and

(c) attachment to a subsequent reaction product of the antigen-binding molecule.

The reporter molecule may be selected from a group including a chromogen, a catalyst, an enzyme, a fluorochrome, a chemiluminescent molecule, a lanthanide ion such as Europium (Eu³⁴), a radioisotope and a direct visual label.

In the case of a direct visual label, use may be made of a colloidal metallic or non-metallic particle, a dye particle, an enzyme or a substrate, an organic polymer, a latex particle, a liposome, or other vesicle containing a signal producing substance and the like.

A large number of enzymes suitable for use as reporter molecules is disclosed in United States Patent Specifications U.S. Pat. No. 4,366,241, U.S. Pat. No. 4,843,000, and U.S. Pat. No. 4,849,338. Suitable enzymes useful in the present invention include alkaline phosphatase, horseradish peroxides, luciferase, β-galactosidase, glucose oxidase, lysozyme, malate dehydrogenase and the like. The enzymes may be used alone or in combination with a second enzyme that is in solution.

Suitable fluorochromes include, but are not limited to, fluorescein isothiocyanate (FITC), tetramethylrhodamine isothiocyanate (TRITC), R-Phycoerythrin (RPE), and Texas Red. Other exemplary fluorochromes include those discussed by Dower et al. (International Publication WO 93/06121). Reference also may be made to the fluorochromes described in U.S. Pat. Nos. 5,573,909 (Singer et al), 5,326,692 (Brinkley et al). Alternatively, reference may be made to the fluorochromes described in U.S. Pat. Nos. 5,227,487, 5,274,113, 5,405,975, 5,433,896, 5,442,045, 5,451,663, 5,453,517, 5,459,276, 5,516,864, 5,648,270 and 5,723,218.

In the case of an enzyme immunoassay, an enzyme is conjugated to the second antibody, generally by means of glutaraldehyde or periodate. As will be readily recognised, however, a wide variety of different conjugation techniques exist which are readily available to the skilled artisan. The substrates to be used with the specific enzymes are generally chosen for the production of, upon hydrolysis by the corresponding enzyme, a detectable colour change. Examples of suitable enzymes include those described supra. It is also possible to employ fluorogenic substrates, which yield a fluorescent product rather than the chromogenic substrates noted above. In all cases, the enzyme-labelled antibody is added to the first antibody-antigen complex. It is then allowed to bind, and excess reagent is washed away. A solution containing the appropriate substrate is then added to the complex of antibody-antigen-antibody. The substrate will react with the enzyme linked to the second antibody, giving a qualitative visual signal, which may be further quantitated, usually spectrophotometrically, to give an indication of the amount of antigen which was present in the sample.

Alternately, fluorescent compounds, such as fluorescein, rhodamine and the lanthanide, europium (EU), may be chemically coupled to antibodies without altering their binding capacity. When activated by illumination with light of a particular wavelength, the fluorochrome-labelled antibody adsorbs the light energy, inducing a state to excitability in the molecule, followed by emission of the light at a characteristic colour visually detectable with a light microscope. The fluorescent-labelled antibody is allowed to bind to the first antibody-antigen complex. After washing off the unbound reagent, the remaining tertiary complex is then exposed to light of an appropriate wavelength. The fluorescence observed indicates the presence of the antigen of interest. Immunofluorometric assays (IFMA) are well established in the art. However, other reporter molecules, such as radioisotope, chemilumine scent or bioluminescent molecules may also be employed.

5.2 Detection of Polynucleotides According to the Invention

In another embodiment, the method for detection comprises detecting expression in a cell of a polynucleotide encoding said polypeptide, fragment, variant or derivative. Expression of the said polynucleotide may be determined using any suitable technique. For example, a labelled polynucleotide encoding a said member may be utilised as a probe in a Northern blot of a RNA extract obtained from the muscle cell. Preferably, a nucleic acid extract from the animal is utilised in concert with oligonucleotide primers corresponding to sense and antisense sequences of a polynucleotide encoding a said member, or flanking sequences thereof, in a nucleic acid amplification reaction such as RT PCR. A variety of automated solid-phase detection techniques are also appropriate. For example, very large scale immobilized primer arrays (VLSIPS™) are used for the detection of nucleic acids as for example described by Fodor et al., (1991, Science 251:767-777) and Kazal et al., (1996, Nature Medicine 2:753-759). The above generic techniques are well known to persons skilled in the art.

6. Screening for Modulators of OKL38

The present invention is predicated in part on the discovery that undesirable downregulation or inactivation of OKL38 is associated with reduction of cell differentiation and promotion of cell proliferation and tumorigenesis. It has also been determined that enhanced expression of OKL38 cDNA leads to enhancement of cell differentiation and to reduction of cell proliferation and tumorigenesis. Accordingly, it is believed that modulation of the level and/or functional activity of OKL38, inclusive of fragments, variants and derivatives of OKL38, or modulation of expression of genes encoding these molecules could, for example, modulate one or more of the above activities. High level expression of OKL38 has been shown in the breast, ovary, kidney, liver, testis, bladder and lung and, in this regard, OKL38 is believed to be involved in modulating the aforementioned activities in these organs.

Modulators contemplated by the present invention includes agonists and antagonists of OKL38 gene expression. Antagonists of OKL38 gene expression include antisense molecules, ribozymes and co-suppression molecules. Agonists include molecules that increase promoter activity or interfere with negative mechanisms. Agonists of OKL38 include molecules that overcome any negative regulatory mechanism. Antagonists of OKL38 polypeptides include antibodies and inhibitor peptide fragments.

The invention therefore provides a method for screening for an agent which modulates one or more of the conditions or activities mentioned above, comprising contacting a preparation comprising a polypeptide as broadly described above or a genetic sequence encoding said polypeptide with a test agent; and detecting a change in the level and/or functional activity of said polypeptide or an expression product of said genetic sequence.

In preferred embodiments, methods are provided for screening for small (non-peptide) inhibitors of OKL38. In this regard, small molecule-based therapies are particularly preferred because such molecules are more readily absorbed after oral administration, have fewer potential antigenic determinants, and/or are more likely to cross the cell membrane than larger, protein-based pharmaceuticals.

Screening for modulatory agents according to the invention can be achieved by any suitable method. For example, the method may include contacting a cell, preferably a mammary epithelial cell, comprising a genetic sequence from which a target protein such as OKL38 can be translated, with an agent suspected of having said modulatory activity and screening for the modulation of that protein, or the modulation of expression of the genetic sequence encoding that protein, or the modulation of the activity or expression of a downstream cellular target of said protein. Detecting such modulation can be achieved utilising techniques including, but not restricted to, Western blotting, ELISA, and RT-PCR.

It will be understood that a genetic sequence from which the target protein of interest is regulated or expressed may be naturally occurring in the cell which is the subject of testing or it may have been introduced into the host cell for the purpose of testing. Further, the naturally-occurring or introduced sequence may be constitutively expressed—thereby providing a model useful in screening for agents which down-regulate expression of an encoded product of the sequence wherein said down regulation can be at the nucleic acid or expression product level—or may require activation—thereby providing a model useful in screening for agents that up-regulate expression of an encoded product of the sequence. Further, to the extent that a polynucleotide is introduced into a cell, that polynucleotide may comprise the entire coding sequence which codes for the target protein or it may comprise a portion of that coding sequence (e.g. a domain such as a protein binding domain) or a portion that regulates expression of a product encoded by the polynucleotide (e.g., a promoter). For example, the promoter that is naturally associated with the genetic sequence may be introduced into the cell, which is the subject of testing. In this regard, where only the promoter is utilised, detecting modulation of the promoter activity can be achieved, for example, by operably linking the promoter to a suitable reporter polynucleotide including, but not restricted to, luciferase, β-galactosidase and CAT. Modulation of expression may be determined by measuring the activity associated with the reporter polynucleotide.

In another example, the subject of detection could be a downstream regulatory target of the target protein, rather than target protein itself or the reporter molecule operably linked to a promoter of a gene encoding a protein the expression of which is regulated by the target protein.

These methods provide a mechanism for performing high throughput screening of putative modulatory agents such as proteinaceous or non-proteinaceous agents comprising synthetic, combinatorial, chemical and natural libraries. These methods will also facilitate the detection of agents which bind either the genetic sequence encoding the target protein or expression product itself or which modulate the expression of an upstream molecule, which subsequently modulates the expression of the genetic sequence encoding the target protein or expression product activity. Accordingly, these methods provide a mechanism of detecting agents, which either directly or indirectly modulate the expression and/or activity of a target protein according to the invention.

In a series of preferred embodiments, the present invention provides assays for identifying small molecules or other compounds (i.e., modulatory agents) which are capable of inducing or inhibiting the expression of OKL38 or OKL38-related genes and proteins. The assays may be performed in vitro using non-transformed cells, immortalised cell lines, or recombinant cell lines. In addition, the assays may detect the presence of increased or decreased expression of OKL38 or other OKL38-related genes or proteins on the basis of increased or decreased mRNA expression (using, for example, the nucleic acid probes disclosed herein), increased or decreased levels of OKL38 or other OKL38-related protein products (using, for example, the anti-OKL38 antigen-binding molecules disclosed herein), or increased or decreased levels of expression of a reporter gene (e.g., β-galactosidase or luciferase) operably linked to a OKL38 5′ regulatory region in a recombinant construct.

Thus, for example, one may culture cells known to express a particular OKL38 and add to the culture medium one or more test compounds. After allowing a sufficient period of time (e.g., 6-72 hours) for the compound to induce or inhibit the expression of OKL38, any change in levels of expression from an established baseline may be detected using any of the techniques described above and well known in the art. In particularly preferred embodiments, the cells are epithelial. Using the nucleic acid probes and/or antigen-binding molecules disclosed herein, detection of changes in the expression of a OKL38, and thus identification of the compound as an inducer or repressor of OKL38 expression, requires only routine experimentation.

In particularly preferred embodiments, a recombinant assay is employed in which a reporter gene such a β-galactosidase or luciferase is operably linked to the 5′ regulatory regions of a OKL38 gene. Such regulatory regions may be easily isolated and cloned by one of ordinary skill in the art in light of the present disclosure of the coding regions of these genes. The reporter gene and regulatory regions are joined in-frame (or in each of the three possible reading frames) so that transcription and translation of the reporter gene may proceed under the control of the Sox18 regulatory elements. The recombinant construct may then be introduced into any appropriate cell type although mammalian cells are preferred, and human cells are most preferred. The transformed cells may be grown in culture and, after establishing the baseline level of expression of the reporter gene, test compounds may be added to the medium. The ease of detection of the expression of the reporter gene provides for a rapid, high throughput assay for the identification of inducers and repressors of the OKL38 gene.

Compounds identified by this method will have potential utility in modifying the expression of OKL38 or other OKL38-related genes in vivo. These compounds may be further tested in the animal models to identify those compounds having the most potent in vivo effects. In addition, as described above with respect to small molecules having OKL38-binding activity, these molecules may serve as “lead compounds” for the further development of pharmaceuticals by, for example, subjecting the compounds to sequential modifications, molecular modelling, and other routine procedures employed in rational drug design.

In another embodiment, a method of identifying agents that promote, augment or otherwise enhance the level and/or functional activity of OKL38 expression is provided. For example, an OKL38 activator can be identified by measuring the ability of a candidate agent to increase OKL38 activity in a cell (e.g., a breast cell, a liver cell etc). In this method, a cell that is capable of expressing OKL38 is exposed to, or cultured in the presence and absence of, the candidate agent and an activity selected from the group consisting of cell proliferation, cell differentiation and tumorigenesis detected or measured. An agent tests positive if it inhibits or protects against cell proliferation or tumorigenesis, or if it promotes, augments or otherwise enhances cell differentiation. For example, enhancement of OKL38 activity or expression could be tested in breast cancer cells or cell lines (e.g., MCF-7 cells) as more fully described infra.

In yet another embodiment, random peptide libraries consisting of all possible combinations of amino acids attached to a solid phase support may be used to identify peptides that are able to bind to OKL38 or to a functional domain thereof. Identification of molecules that are able to bind to OKL38 may be accomplished by screening a peptide library with a recombinant soluble OKL38. The OKL38 may be purified, recombinantly expressed or synthesised by any suitable technique.

To identify and isolate the peptide/solid phase support that interacts and forms a complex with OKL38, it is necessary to label or “tag” OKL38. OKL38 may be conjugated to any suitable reporter molecule, including enzymes such as alkaline phosphatase and horseradish peroxidase and fluorescent reporter molecules such as fluorescein isothyiocynate (FITC), phycoerythrin (PE) and rhodamine. Conjugation of any given reporter molecule, with OKL38, may be performed using techniques that are routine in the art. Alternatively, OKL38 expression vectors may be engineered to express a chimeric OKL38 containing an epitope for which an antigen-binding molecule exists. The epitope specific antigen-binding molecule may be tagged using methods well known in the art including labelling with enzymes, fluorescent dyes or coloured or magnetic beads as for example described in Section 5.

The “tagged” OKL38 conjugate is incubated with the random peptide library for 30 minutes to one hour at 22° C. to allow complex formation between OKL38 and peptide species within the library. The library is then washed to remove any unbound OKL38 protein. If OKL38 has been conjugated to alkaline phosphatase or horseradish peroxidase the whole library is poured into a petri dish containing a substrate for either alkaline phosphatase or peroxidase, for example, 5-bromo-4-chloro-3-indoyl phosphate (BCIP) or 3,3′,4,4″-diamnobenzidine (DAB), respectively. After incubating for several minutes, the peptide/solid phase-OKL38 complex changes colour, and can be easily identified and isolated physically under a dissecting microscope with a micromanipulator. If a fluorescent-tagged OKL38 molecule has been used, complexes may be isolated by fluorescent activated sorting. If a chimeric OKL38 protein expressing a heterologous epitope has been used, detection of the peptide/OKL38 complex may be accomplished by using a labelled epitope specific antigen-binding molecule. Once isolated, the identity of the peptide attached to the solid phase support may be determined by peptide sequencing.

7. Method of Modulating a OKL38-Related Activity

The invention therefore provides a method for modulating at least one activity selected from the group consisting of cell differentiation, cell proliferation and tumorigenesis, comprising contacting a cell with an agent for a time and under conditions sufficient to modulate the level and/or functional activity of a polypeptide as broadly described above.

In a preferred embodiment, the agent increases the level and/or functional activity of OKL38. Any suitable OKL38 inducers or stabilising/activating agents may be used in this regard and these can be identified or produced by methods for example disclosed in Section 6.

The invention also encompasses a method for modulating at least one activity selected from the group consisting of cell differentiation, cell proliferation and tumorigenesis, comprising contacting a cell with a member selected from the group consisting of a polypeptide comprising the amino acid sequence set forth in SEQ ID NO: 2, a fragment of said polypeptide, a variant of said polypeptide, a variant of said polypeptide fragment, a derivative of said polypeptide, a derivative of said polypeptide fragment, a polynucleotide encoding said polypeptide, a fragment of said polynucleotide, a variant of said polynucleotide and a variant of said polynucleotide fragment, for a time and under conditions sufficient to modulate said activity.

8. Compositions

The polypeptides, fragments, variants and derivatives described in Section 2, the polynucleotides and polynucleotide variants described in Section 3, and the modulatory agents described in Section 6 and 7 (therapeutic agents) can be used as actives for the treatment or prophylaxis of conditions associated with cell proliferation and/or tumorigenesis. In a preferred embodiment, the condition is a cancer or tumour of an organ selected from the group consisting of breast, liver, testis, kidney and lung. In an alternate embodiment, the condition is a cancer or tumour of the prostate. These therapeutic agents can be administered to a patient either by themselves, or in pharmaceutical compositions where they are mixed with a suitable pharmaceutically acceptable carrier.

Accordingly, the invention also provides a composition for modulating an activity selected from cell proliferation, cell differentiation and tumorigenesis, comprising an agent selected from the group consisting of a polypeptide, fragment, variant or derivative as broadly described above, a polynucleotide from which said polypeptide, fragment, variant or derivative can be translated, and a modulatory agent that modulates the level and/or functional activity of OKL38.

The invention also contemplates a composition for treatment and/or prophylaxis of cancer or tumour, preferably a cancer or tumour of the breast. The composition broadly comprises an agent selected from the group consisting of a polypeptide, fragment, variant or derivative as broadly described above, a polynucleotide from which said polypeptide, fragment, variant or derivative can be translated, and a modulatory agent that enhances the level and/or functional activity of OKL38, and optionally together with a pharmaceutically acceptable carrier.

Depending on the specific conditions being treated, therapeutic agents may be formulated and administered systemically or locally. Techniques for formulation and administration may be found in “Remington's Pharmaceutical Sciences,” Mack Publishing Co., Easton, Pa., latest edition. Suitable routes may, for example, include oral, rectal, transmucosal, or intestinal administration; parenteral delivery, including intramuscular, subcutaneous, intramedullary injections, as well as intrathecal, direct intraventricular, intravenous, intraperitoneal, intranasal, or intraocular injections. For injection, the therapeutic agents of the invention may be formulated in aqueous solutions, preferably in physiologically compatible buffers such as Hanks' solution, Ringer's solution, or physiological saline buffer. For transmucosal administration, penetrants appropriate to the barrier to be permeated are used in the formulation. Such penetrants are generally known in the art. Intra-muscular and subcutaneous injection is appropriate, for example, for administration of immunogenic compositions, vaccines and DNA vaccines.

The agents can be formulated readily using pharmaceutically acceptable carriers well known in the art into dosages suitable for oral administration. Such carriers enable the compounds of the invention to be formulated in dosage forms such as tablets, pills, capsules, liquids, gels, syrups, slurries, suspensions and the like, for oral ingestion by a patient to be treated. These carriers may be selected from sugars, starches, cellulose and its derivatives, malt, gelatine, talc, calcium sulphate, vegetable oils, synthetic oils, polyols, alginic acid, phosphate buffered solutions, emulsifiers, isotonic saline, and pyrogen-free water.

Pharmaceutical compositions suitable for use in the present invention include compositions wherein the active ingredients are contained in an effective amount to achieve its intended purpose. The dose of agent administered to a patient should be sufficient to effect a beneficial response in the patient over time such as a reduction in the symptoms associated with the cancer or tumour or an increase in protection against tumorigenesis. The quantity of the agent(s) to be administered may depend on the subject to be treated inclusive of the age, sex, weight and general health condition thereof. In this regard, precise amounts of the agent(s) for administration will depend on the judgement of the practitioner. In determining the effective amount of the agent to be administered in the treatment or prophylaxis of the cancer or tumour, the physician may evaluate tissue levels of a polypeptide, fragment, variant or derivative of the invention, and progression of the cancer or tumour. In any event, those of skill in the art may readily determine suitable dosages of the therapeutic agents of the invention.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances that increase the viscosity of the suspension, such as sodium carboxymethyl cellulose, sorbitol, or dextran. Optionally, the suspension may also contain suitable stabilisers or agents, which increase the solubility of the compounds to allow for the preparation of highly concentrated solutions.

Pharmaceutical preparations for oral use can be obtained by combining the active compounds with solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as., for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Such compositions may be prepared by any of the methods of pharmacy but all methods include the step of bringing into association one or more therapeutic agents as described above with the carrier which constitutes one or more necessary ingredients. In general, the pharmaceutical compositions of the present invention may be manufactured in a manner that is itself known, e.g., by means of conventional mixing, dissolving, granulating, dragee-making, levigating, emulsifying, encapsulating, entrapping or lyophilising processes.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterise different combinations of active compound doses.

Pharmaceutical which can be used orally include push-fit capsules made of gelatin, as well as soft, sealed capsules made of gelatin and a plasticiser, such as glycerol or sorbitol. The push-fit capsules can contain the active ingredients in admixture with filler such as lactose, binders such as starches, and/or lubricants such as talc or magnesium stearate and, optionally, stabilisers. In soft capsules, the active compounds may be dissolved or suspended in suitable liquids, such as fatty oils, liquid paraffin, or liquid polyethylene glycols. In addition, stabilisers may be added.

Dosage forms of the therapeutic agents of the invention may also include injecting or implanting controlled releasing devices designed specifically for this purpose or other forms of implants modified to act additionally in this fashion. Controlled release of an agent of the invention may be effected by coating the same, for example, with hydrophobic polymers including acrylic resins, waxes, higher aliphatic alcohols, polylactic and polyglycolic acids and certain cellulose derivatives such as hydroxypropylmethyl cellulose. In addition, controlled release may be effected by using other polymer matrices, liposomes and/or microspheres.

Therapeutic agents of the invention may be provided as salts with pharmaceutically compatible counterions. Pharmaceutically compatible salts may be formed with many acids, including but not limited to hydrochloric, sulfuric, acetic, lactic, tartaric, malic, succinic, etc. Salts tend to be more soluble in aqueous or other protonic solvents that are the corresponding free base forms.

For any compound used in the method of the invention, the therapeutically effective dose can be estimated initially from cell culture assays. For example, a dose can be formulated in animal models to achieve a circulating concentration range that includes the IC50 as determined in cell culture (e.g., the concentration of a test agent, which achieves a half-maximal inhibition or enhancement of OKL38 activity). Such information can be used to more accurately determine useful doses in humans.

Toxicity and therapeutic efficacy of such therapeutic agents can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit large therapeutic indices are preferred. The data obtained from these cell culture assays and animal studies can be used in formulating a range of dosage for use in human. The dosage of such compounds lies preferably within a range of circulating concentrations that include the ED50 with little or no toxicity. The dosage may vary within this range depending upon the dosage form employed and the route of administration utilised. The exact formulation, route of administration and dosage can be chosen by the individual physician in view of the patient's condition. (See for example Fingl et al., 1975, in “The Pharmacological Basis of Therapeutics”, Ch. 1 p1).

Dosage amount and interval may be adjusted individually to provide plasma levels of the active agent which are sufficient to maintain OKL38-enhancement effects. Usual patient dosages for systemic administration range from 1-2000 mg/day, commonly from 1-250 mg/day, and typically from 10-150 mg/day. Stated in terms of patient body weight, usual dosages range from 0.02-25 mg/kg/day, commonly from 0.02-3 mg/kg/day, typically from 0.2-1.5 mg/kg/day. Stated in terms of patient body surface areas, usual dosages range from 0.5-1200 mg/m²/day, commonly from 0.5-150 mg/m²/day, typically from 5-100 mg/m²/day.

Alternately, one may administer the compound in a local rather than systemic manner, for example, via injection of the compound directly into a tissue, which is preferably a breast tissue, often in a depot or sustained release formulation.

Furthermore, one may administer the drug in a targeted drug delivery system, for example, in a liposome coated with tissue-specific antibody. The liposomes will be targeted to and taken up selectively by the tissue.

In cases of local administration or selective uptake, the effective local concentration of the agent may not be related to plasma concentration.

Thus, the present invention also contemplates a method of gene therapy of an animal, and preferably of a mammal. Such a method utilises a gene therapy construct which includes an isolated OKL38 polynucleotide ligated into a gene therapy vector which provides one or more regulatory sequences that direct expression of said polynucleotide in said animal.

Typically, gene therapy vectors are derived from viral DNA sequences such as adenovirus, adeno-associated viruses, herpes-simplex viruses and retroviruses. Suitable gene therapy vectors currently available to the skilled person may be found, for example, in Robbins et al., 1998, Trends Biotechnol. 16: 35. Administration of the gene therapy construct to said mammal, preferably a human, may include delivery via direct oral intake, systemic injection, or delivery to selected tissue(s) or cells, or indirectly via delivery to cells isolated from the mammal or a compatible donor. An example of the latter approach would be stem-cell therapy, wherein isolated stem cells having potential for growth and differentiation are transfected with the vector comprising the OKL38 nucleic acid. The stem cells are cultured for a period and then transferred to the mammal being treated.

Delivery of said gene therapy construct to cells or tissues of said mammal or said compatible donor may be facilitated by microprojectile bombardment, liposome mediated transfection (e.g., lipofectin or lipofectamine), electroporation, calcium phosphate or DEAE-dextran-mediated transfection, for example. A discussion of suitable delivery methods may be found in Chapter 9 of CURRENT PROTOCOLS IN MOLECULAR BIOLOGY (Eds. Ausubel et al; John Wiley & Sons Inc., 1997 Edition), for example, which is herein incorporated by reference. For example, an OKL38 nucleic acid may be introduced into a cell to enhance the ability of that cell to promote cell differentiation or to decrease or reduce its ability to promote cell proliferation.

In an alternate embodiment, a polynucleotide encoding a modulatory agent of the invention may be used as a therapeutic or prophylactic composition in the form of a “naked DNA” composition as is known in the art. For example, an expression vector comprising said polynucleotide operably linked to a regulatory polynucleotide (e.g. a promoter, transcriptional terminator, enhancer etc) may be introduced into an animal, preferably a mammal, where it causes production of a modulatory agent in vivo, preferably breast tissue.

The step of introducing the expression vector into a target cell or tissue will differ depending on the intended use and species, and can involve one or more of non-viral and viral vectors, cationic liposomes, retroviruses, and adenoviruses such as, for example, described in Mulligan, R. C., (1993 Science 260: 926-932. Such methods can include, for example:

A. Local application of the expression vector by injection (Wolff et al., 1990, Science 247: 1465-1468), surgical implantation, instillation or any other means. This method can also be used in combination with local application by injection, surgical implantation, instillation or any other means, of cells responsive to the protein encoded by the expression vector so as to increase the effectiveness of that treatment. This method can also be used in combination with local application by injection, surgical implantation, instillation or any other means, of another factor or factors required for the activity of said protein.

B. General systemic delivery by injection of DNA, (Calabretta et al., 1993, Cancer Treat. Rev. 19: 169-179), or RNA, alone or in combination with liposomes (Zhu et al., 1993, Science 261: 209-212), viral capsids or nanoparticles (Bertling et al., 1991, Biotech. Appl. Biochem. 13: 390-405) or any other mediator of delivery. Improved targeting might be achieved by linking the polynucleotide/expression vector to a targeting molecule (the so-called “magic bullet” approach employing, for example, an antigen-binding molecule), or by local application by injection, surgical implantation or any other means, of another factor or factors required for the activity of the protein encoded by said expression vector, or of cells responsive to said protein.

C. Injection or implantation or delivery by any means, of cells that have been modified ex vivo by transfection (for example, in the presence of calcium phosphate: Chen et al., 1987, Mole. Cell Biochem. 7: 2745-2752, or of cationic lipids and polyamines: Rose et al., 1991, BioTech. 10: 520-525), infection, injection, electroporation (Shigekawa et al., 1988, BioTech. 6: 742-751) or any other way so as to increase the expression of said polynucleotide in those cells. The modification can be mediated by plasmid, bacteriophage, cosmid, viral (such as adenoviral or retroviral; Mulligan, 1993, Science 260: 926-932; Miller, 1992, Nature 357: 455-460; Salmons et al., 1993, Hum. Gen. Ther. 4: 129-141) or other vectors, or other agents of modification such as liposomes (Zhu et al., 1993, Science 261: 209-212), viral capsids or nanoparticles (Bertling et al., 1991, Biotech. Appl. Biochein. 13 390-405), or any other mediator of modification. The use of cells as a delivery vehicle for genes or gene products has been described by Barr et al., 1991, Science 254: 1507-1512 and by Dhawan et al., 1991, Science 254 1509-1512. Treated cells can be delivered in combination with any nutrient, growth factor, matrix or other agent that will promote their survival in the treated subject.

In order that the invention may be readily understood and put into practical effect, particular preferred embodiments will now be described by way of the following non-limiting example.

### EXAMPLES

### Example 1

mRNA Differential Display

Differential display was performed using RNA from pregnant mammary gland according to the protocol supplied with the RNAmapJ kit (GeneHunter Corp., Nashville, Tenn.). Briefly, 5 μg of DNAse I-treated total RNA were reverse transcribed with T₁₂Mⁿ (where n may be G, A, T, or C), followed by PCR amplification in the presence of [α-³²P]dATP (NEN) using the corresponding T₁₂Mₙ primer, downstream, and one arbitrary primers supplied with the kit, AP₁-AP₅, upstream. The PCR-amplified fragments were separated on 6% denaturing polyacrylamide gel. The gel was dried and exposed to Kodak XAR film, and cDNAs representing differential expressed mRNAs were excised from the dried gels and reamplified cDNA fragments were used to as probes in Northern blotting to verify their differential expression in mammary gland. The differential 450 bp probe was used to screen human ovarian cDNA library as described (18). The isolated human cDNAs were sequenced by the Sanger dideoxy chain determination method and their nucleotide sequences were compared with those deposited in the GenBank and EMBL data banks.

Among 18 differential bands obtained using the above protocol, one band of approximately 450 bp was novel as determined by sequence analysis. The differential expression of this particular cDNA was confirmed by Northern blotting. The 450 bp band detected an approximate 1.6 kb mRNA species.

### Example 2

Nucleic Acid Sequence Analysis

To obtain the full-length cDNA, human ovarian cDNA library was screened using this 450-bp probe. Eight positive clones were isolated and clone-purified. One clone containing an approximate 1.6-kb insert was isolated and sequenced by the Sanger dideoxy chain determination method.

Comparison of the nucleotide sequence against the non-redundant nucleotide database of GenBank established this 1.6-kb cDNA was novel. Blast search revealed no significant homology with any known sequences. This cDNA (GenBank accession no AF191740) contained 1607 bp and was full-length cDNA. An initiator ATG codon (position 127) is followed by a single open reading frame of 317 amino acids with a calculated molecular weight of 34.5 kDa. The open reading frame ended with a TGA terminator codon at position 1078 followed by 529 nucleotides in the 3′ untranslated region.

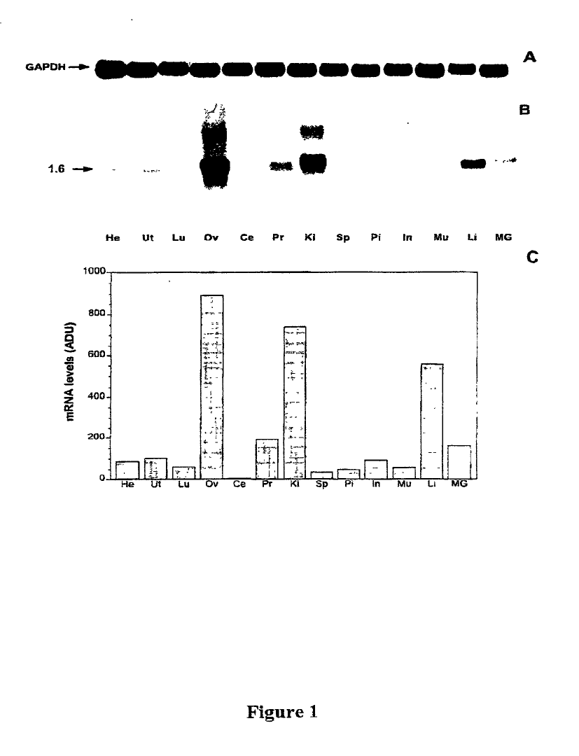

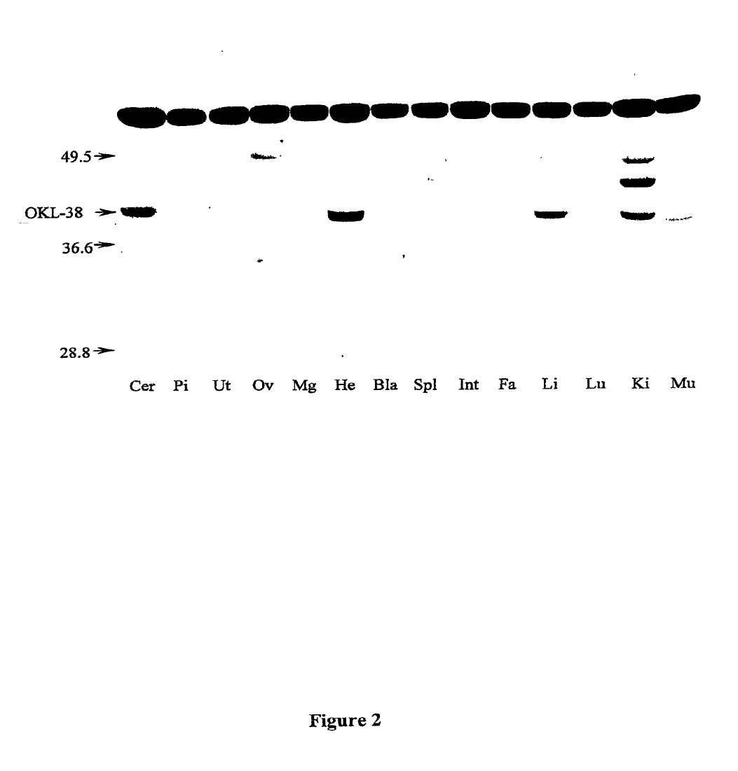

To determine the distribution of 1.6-kb transcripts, Northern blot analysis was performed using poly “A” RNA derived from various tissues of mature female rats. The bank of approximate 1.6-kb were observed in all tissues with the highest levels were seen in the ovary, kidney and liver (FIG. 1). Minor bands of approximate 3.6- and 4-kb were also observed. Western blotting using rabbit polyclonal antibody against OKL38 protein recognised a 38 kDa protein in most of the tissues examined with the highest levels found in the heart, cerebellum, kidney, lung, testis, ovary and liver (FIG. 2). The protein was designated OKL38.

### Example 3

Animals and Drug Administration.

Animal experiments were approved by local Animal Care Committee. Female Sprague-Dawley rats, 50 days old at the beginning of the experiments, were obtained from Charles River, Quebec. We have assessed the in vivo effects of pure anti-estrogen ICI 182780, tamoxifen, estrogens on OKL38 gene expression in the mammary gland of a rat model. To study the effect of estradiol on OKL38 gene expression, groups of rats were implanted with either 0.5 cm and 1.0 cm silastic tubes (0.04 in ID, Dow corning, Michigan) containing 17-β estradiol (Sigma) on the back of their neck. Control rats experienced the same surgical implantation with empty silastic tubes. Based on previous published work, the released rate of 17 β-estradiol from silastic implants was documented to be 2.4 μg/cm/day. Tamoxifen (Sigma) was dissolved in castor oil at a concentration of 1 mg/ml. Female rats were daily received 400 μg/per kg BW. Preformulated ICI 182780 (Zeneca Pharmaceuticals) was supplied at a concentration of 50 mg/mL in castor oil solution. Female Sprague-Dawley rats were weekly received, by subcutaneous injection of either castor oil alone or 1 and 1.5 mg of ICI per kg BW for 3 weeks. At the end of the experiments, animals were sacrificed by carbon dioxide exposure. The mammary tissue was excised, trimmed, and snap-frozen in liquid nitrogen and stored at −70° C. for RNA extraction.

To study changes in OKL38 expression during pregnancy and lactation of normal rats, pregnant rats were sacrificed on days 0, 4, 10, 16 and 21 of pregnancy and day 3 of lactation. Mating dates were established from the appearance of vaginal plugs. Day 1 of pregnancy was the day on which a plug was observed. The animals were sacrificed and the mammary gland collected as described above.

### Example 4

OKL38 Expression

To investigate the changes of OKL38 expression during pregnancy and lactation, poly “A” RNA and proteins derived from mammary gland at different stages of pregnancy were analysed by Northern and Western blotting, respectively.

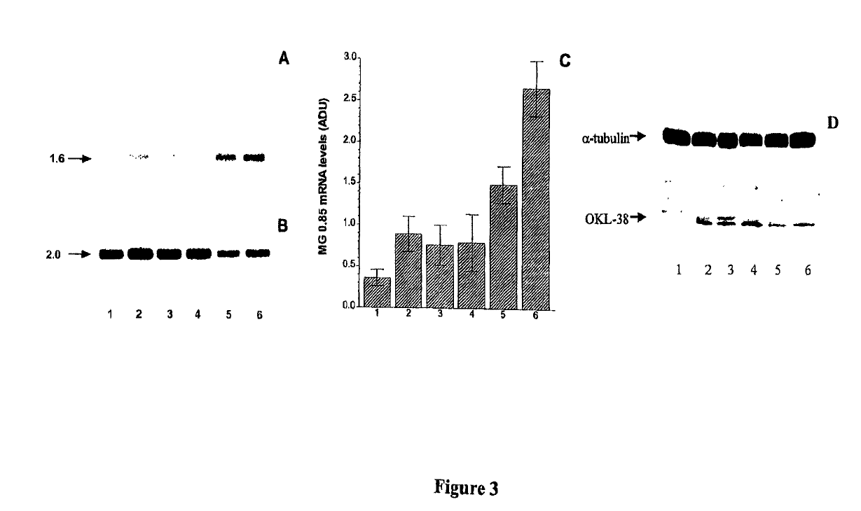

Poly “A” RNA was isolated from indicated tissues of female rats as described (22). Northern blots were performed on poly “A” RNA or total RNA and blots were hybridised with human OKL38 or human GAPDH (ATCC) cDNAs as previously described (22). Messenger RNA levels were determined by densitometric scanning of autoradiograms. As shown in FIG. 3, the levels of OKL38 mRNA were very low in mammary tissue of non-pregnant rats. Following pregnancy, the OKL38 mRNA increased rapidly and maximal OKL38 expression was observed during lactation.

For Western blot analysis, mammary tissue was homogenised in buffer containing 1 mM CaCl₂ 1 mM MgCl₂, 1% NP-40, 1 μg/mL leupeptin, 1 μg/mL aprotinin, 1 μM PMSF, and 100 μM NaVO₄. Cells were lysed in the above buffer. Cell lysate was used to determine changes in the levels of OKL38 by Western blotting were was described (21). Blots were incubated with rabbit anti-OKL38 (1:500 dilution) and horseradish peroxidase-conjugated donkey anti-rabbit secondary antibody (1:7500). Blots were visualised with a chemiluminescent detection system (ECL, Amersham) and exposed to film for 10 sec to 45 sec.

Western blot analysis revealed that OKL38 protein already peaked during early pregnancy and remained throughout pregnancy and lactation (FIG. 3D).

### Example 5

Effect of hCG

To study if human chorionic gonadotropin (hCG), a known mammary differentiating agent, was capable of increasing OKL38 gene expression, female rats were injected with hCG in a manner such as to simulate levels seen at time of lactation (19). Briefly, rats were inoculated by intraperitoneal injection with doses of 10, 20 and 40 UI of hCG/day in 200 μL of PBS for 21 days in a manner such as to simulate levels seen at time of lactation as described (19). Control rats were administered 200 μL PBS. After the last injection, the rats were allowed to rest for an additional 7 days and at the end of the experiment they were sacrificed. Breast tissue was assayed for OKL38 mRNA Northern blotting.

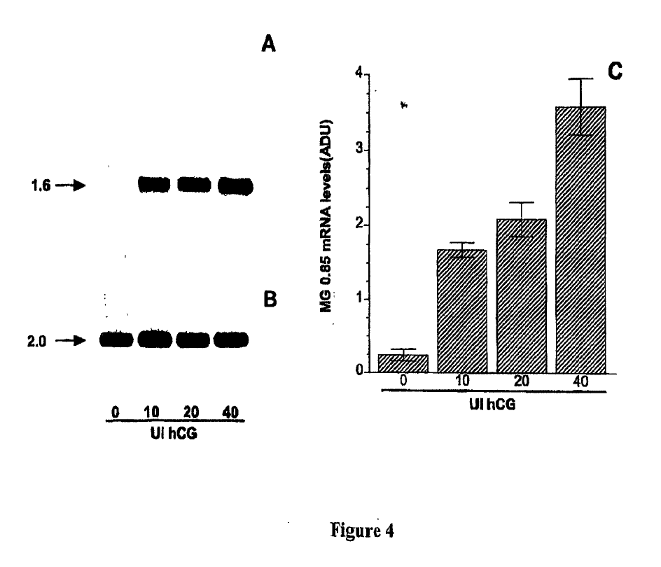

FIG. 4 shows that hCG induced OKL38 gene expression in a dose dependent manner. The expression is significantly increased above baseline at the time of the physiological changes (specifically maximal breast epithelial differentiation) associated with pregnancy and lactation. This provides strong evidence for the induction of OKL38 expression by hCG. A strong relationship between onset of differentiation, inhibition of proliferation, and onset of OKL38 expression is observed.

### Example 6

Effect of Antiestrogens

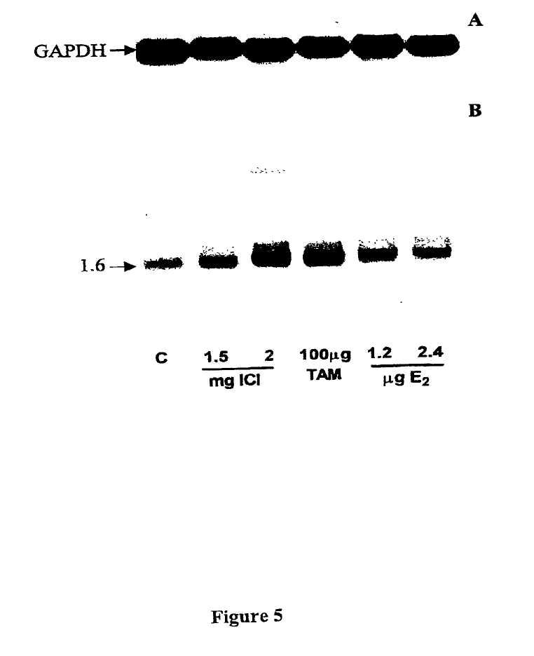

To determine the effects of antiestrogens on OKL38 expression in vivo, female rats treated with vehicle, tamoxifen, 17-β Estradiol or ICI 182780. As shown in FIG. 5, both a pure antiestrogen ICI 182780 and tamoxifen significantly upregulated OKL38 gene expression (p<0.05) while estradiol had no effect.

### Example 7

Induction of Mammary Tumours by DMBA.

Since OKL38 is highly expressed in breast tissue during pregnancy, OKL38 expression was studied in neoplastic tissue, using the rat DMBA-induced mammary tumour experimental system (20).

Standard DMBA-induced mammary tumour experimental model (20) was used to study the expression of OKL38 gene during pregnancy. Mammary carcinomas were induced by a single intragastric administration of 20 mg dimethylbenz(A)anthracene (DMBA, Sigma Chemical Co., St. Louis, Mo.) in 1 mL peanut oil at 50-52 days of age. This standard procedure yields palpable (>0.5 cm) tumours in about 75% of animals by day 80 following carcinogen administration. Rats bearing DMBA-induced breast tumours were mated. Mating dates were established from the appearance of vaginal plugs. Pregnant rats were sacrificed on day 16 of pregnancy and tumours were collected.

To investigate OKL38 gene expression in human breast cancer cell lines, MCF-7, T47D, ZR75, MDA-231, Hs578T, and HBL-100 were grown to 90% confluence. Poly “A” RNA was purified and Northern blotting was performed to determine the levels of OKL38 mRNA.

To study the effects of taxol, doxorubicin and cisplatin on OKL38 gene expression, human MCF-7 breast cancer cells were grown and treated with the indicated concentrations of drugs for 48 h. Cells were harvest for OKL38 mRNA determination by Northern blotting.

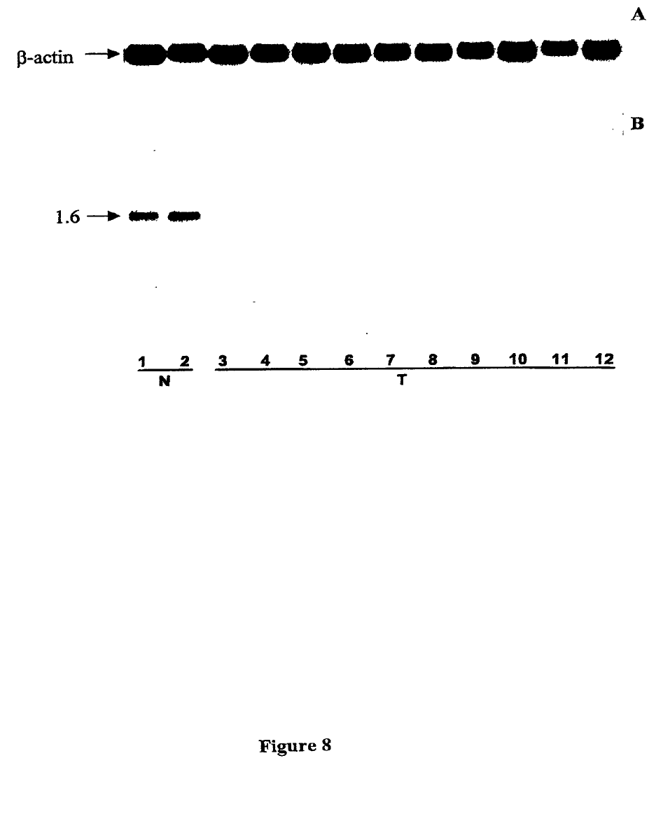

FIG. 8 shows the results of an experiment where animals were exposed to DMBA, tumours were allowed to appear, and rats were permitted to become pregnant. In lactating rats bearing DMBA-induced tumours, the OKL38 expression seen in normal mammary gland was abundant relative to that seen in most mammary ductal neoplasms. OKL38 levels shared similar pattern as determined by Western blotting (data not shown).

### Example 8

OKL38 Expression in Breast Cancer Cells

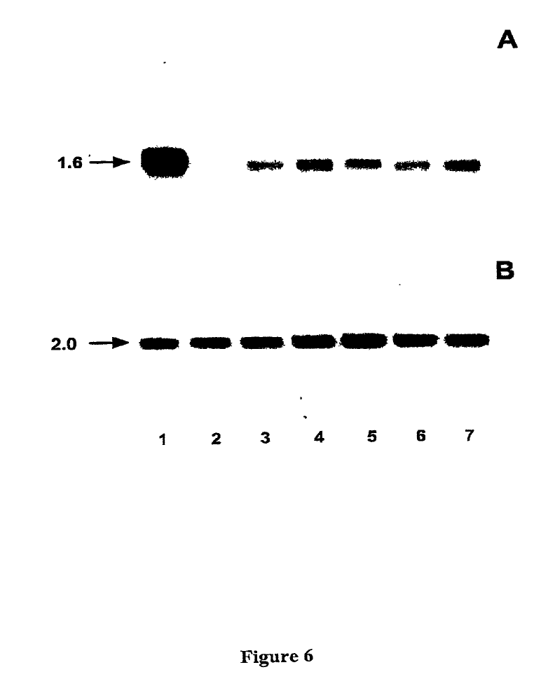

To determine the OKL38 gene expression in human breast cancer cells, poly “A” RNA derived from various cell lines was analysed by Northern blot analysis. FIG. 6 shows that although the OKL38 mRNA was much lower than that seen in breast tissue, OKL38 transcripts were detected in all cell lines. Despite detectable OKL38 mRNA, OKL38 protein in these cell lines was barely detected as determined by Western blotting (data not shown).

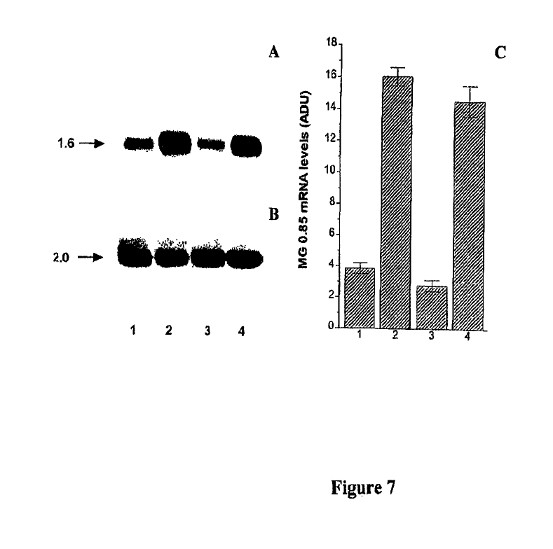

OKL38 gene expression in cultures of human MCF-7 breast cancel cells was significantly induced by taxol and doxorubicin (p<0.01) which are known to inhibit MCF-7 breast cancer cell proliferation (FIG. 7).

From the foregoing, it is possible that loss of OKL38 production is required for changes from normal to malignant state and also confers the growth advantage over normal cells. It is believed that the hormonal changes associated with pregnancy are associated with upregulation of OKL38 expression in vivo, but this normal induction of expression does not take place in neoplastic breast tissue. It is possible, therefore, that the neoplastic progression itself is associated with reduction in OKL38 expression. The molecular mechanisms responsible for OKL38 inactivation in DMBA-induced breast tumours are not known. However, it is possible that absence of, or undetectable OKL38 expression may be in certain tumours might be a result of genetic alterations such as deletion, mutation, or inappropriate hypermethylation (27-29).

### Example 9

MCF-7 Cell Stable Transfectant Cell Lines

The entire coding region of OKL38 cDNA was cloned into mammalian expression vector pcDNA3.1. MCF-7 cells were seeded at 2×10⁵ in 100 mm culture dishes in 90% α-MEM (Life Technologies, Inc) containing 10% FCS with Garamycine 24 h prior to transfection. Cells were transfected with 5 μg of full-length OKL38 cDNA (pcDNA3.1-OKL38) or pDNA3.1 control plasmid DNA and 28 μL of Lipofectamine™ reagent (Life Technologies) following recommendations of the manufacturer. Forty-eight hours following transfection, cells were split 1:10 and replaced with growth medium containing 800 μg/mL G418 (Calbiochem, La Jolla, Calif.). After 4 weeks, clones were isolated, expanded and assayed for OKL38 expression by Western and Northern blot analyses.

Differences in OKL38 gene expression were analysed by Student's t-test. Differences in cell number and tumour number between parental lines and transfectants were tested using the Mann Whitney U-test.

Cell number refers to mean cell number counted by hemocytometer 8 days after seeding 2.5×10⁴ cells in wells containing α-MEM supplemented with 10% foetal calf serum. Means were determined from quadruplicate replicate wells and in no case did standard deviation exceed 15% of the mean value.

In vivo tumour formation was assayed using 4-8 week old athymic nude mice (CD1 nu/nu, Charles River). Each cell line was assayed in four mice, and each mouse received an injection of 5×10⁶ cells into an inframammary fat pad, and another identical injection of the same cell line into a contralateral fat pad. Thirty days after injection, animals were inspected for grossly visible tumours.

### Example 10

Generation of an OKL38 Antibody

Synthetic peptides corresponding to predicted amino acids 243 to 267 of the human OKL38 (N- Arg Glu Gln Ser Ile Leu Ser Pro Ser Pro Tyr Glu Gly Tyr Arg Ser Leu Pro Arg His Gln Leu Leu Cyc Phe -C) were synthesised and coupled to preactivated keyhole limpet hemocyanin (Sheldon Biotechnology, Montréal, Quebéc). Rabbit polyclonal antibodies were produced according to standard protocols. Affinity purified serum following the sixth boost was used in these studies.

### Example 11

OKL38 is a Tumour Suppressor Gene

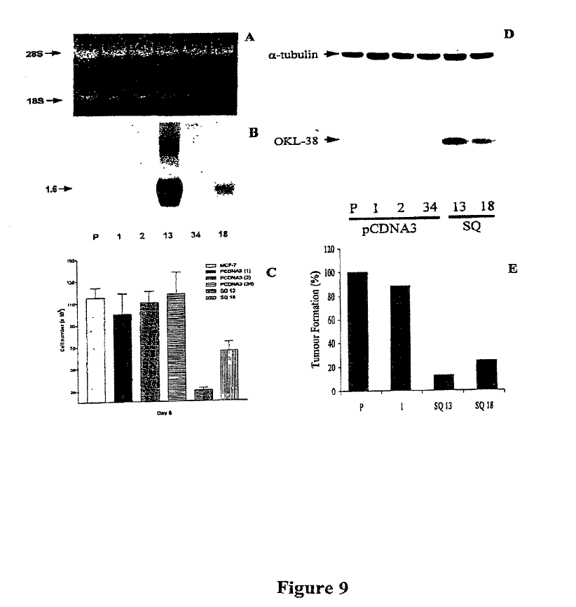

To test the hypothesis that the gene encoding OKL38 is a tumour suppressor gene, human breast cancel cells were transfected with an expression vector for full-length OKL38 cDNA (pcDNA3.1-OKL38) according to Example 9, and the phenotype of transfectants expressing OKL38 was compared with that of controls. FIG. 9B shows high levels of expression of OKL38 mRNA (˜1.6 kb) in representative transfected cell lines SQ13 and SQ 18, but the absence of expression in untransfected MCF7 cells and the mock-transfected cell line. Western blotting with a polyclonal anti-OKL38 antiserum prepared according to Example 10 was used to detect OKL38-related proteins in the various clones. A 38-kDa protein was detected only in cells transfected with pcDNA3.1-OKL38 (FIG. 9C).

Proliferative behaviour of the clones was evaluated by determining cell number on plastic dishes after 8 days of incubation. The number of cells was significantly less (p<0.05, Mann-Whitney U-test) in OKL38-expressing transfectants than in controls (FIG. 9D).

To determine if over-expression of OKL38 leads to reduction in tumour formation, in vivo tumorigenicity was performed in nude mice. As shown in FIG. 9E, the rate of tumour formation following injection of MCF7 cells was 100% (8 of 8), that of pcDNA3.1-1 mock-transfected cells 88% (7 of 8), and that of SQ13 and SQ18 transfected cells 12.5% (1 of 8) and 25% (2 of 8), respectively. Thus, increasing OKL38 levels following transfection leads to a reduction in cellular growth and tumour formation in nude mice suggesting OKL38 plays an important role in growth regulation and tumorigenesis.

### Example 12

Genomic Cloning of Human OKL38.

Genomic DNA from the whole placenta from a 32-yr-old Caucasian was used to construct a cosmid library. Genomic DNA fragments were cloned into the BamHI site of the pWE15 vector (Clonetech Cat. #: HL1095m).

The cosmid library was spread onto culture plates and incubated overnight at 37° C. Bacterial colonies were lifted in duplicate on to the nitrocellulose membranes and air dried. Bacteria were lysed in 0.5 M NaOH for 5 min, neutralised in 1 M Tris-HCl (pH 7.5) for 5 min, followed by 5 min in 1 M Tris-HCl (pH 7.5) containing 1.5 M NaCl. The membranes were scrubbed in 2×SSC and baked at 80° C. for 2 hours. Prehybridisation and hybridisation were carried out as previously described (36). The full length of human OKL38 cDNA was labelled with [α-³²P]dCTP (RediPrime™ random primer labelling kit, Amersham). After hybridisation with the probe for 24 h at 42° C. The membranes were washed with solution A (2×SSC; 0.1% SDS), Solution B (0.5×SSC; 0.1% SDS, Solution C (0.1×SSC; 0.1% SDS) twice each before autoradiography.

Positive clones for OKL38 were digested with NotI, NotI/HindIII, NotI/PstI and NotI/SacI and digested products was separated on an agarose gel (0.8%) by electrophoresis. Fragments ranging from 1.0 Kb to 6 Kb were purified using Qiaquick™ Gel Extraction kit (Qiagen) and cloned into pBluescript® II SK vector for sequencing using ABI Prism® 377 DNA Sequencer (Perkin Elmer, CA) with Big Dye™ terminator mix. The sequences obtained were screened with VecScreen™ (NCBI) and contiged using SeqMan™ II (Dnastar).

### Example 13

Distribution of OKL38 Transcripts in Human Tissues

To determine the distribution of OKL38 transcripts in human tissues, Northern blot analysis was performed using poly “A” RNA derived from various human tissues. The human poly-A RNA blots were obtained from Invitrogen (BioChain Institute, USA). Each lane contained 2 μg poly “A” RNA from different human tissues. Blots were hybridised with human OKL38 or human GAPDH (ATCC) cDNAs as previously described (34).

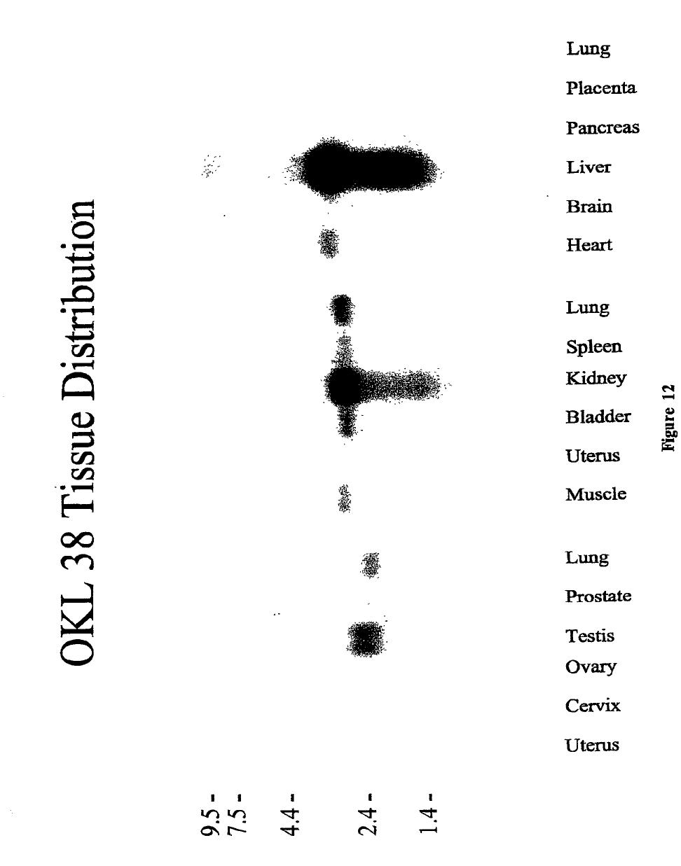

Tissue-specific expression of OKL38 in human tissues shows high expression in the testes, kidneys, lungs and most highly in the liver (see FIG. 12).

### Example 14

Loss of One Copy of OKL38 in Breast Tumours

To determine whether there are any gross changes in the OKL38 gene (or loss of heterozygosity) in breast tumours, 6 breast cancer specimens were examined, which had paired normal breast tissue. The specimens were obtained from surgically biopsied breast tissue which was frozen in liquid nitrogen or cooled in isopentane within 30-45 minutes of excision, and stored at −80° C. until extraction procedures were performed. DNA specimens were digested with enzymes and analysed by Southern blot. Digested patterns of tumour DNA were compared with the paired normal breast DNA.

Genomic DNA was prepared from mammary tissue as described (36). For the analysis of the changes in OKL38 gene, 20 μg genomic DNA was digested with 10 units/μg PstI enzyme in buffers as recommended by the suppliers (Pharmacia) for 16-18 h. To monitor complete cleavage ΦX174 DNA was added as a standard to each digest and the digest was examined for complete Φ174 DNA digestion by gel electrophoresis and ethidium bromide staining. Digested DNA samples were subjected to gel electrophoresis on a 1% agarose gel in TPE buffer (37) at 1.2 V/cm for 20 h. DNA was transferred onto a charged nylon membrane (Zeta-probe™, Bio-Rad) in 0.4 M NaOH according to the supplier's instructions. Prehybridisation and hybridisation were carried out as previously described (36).

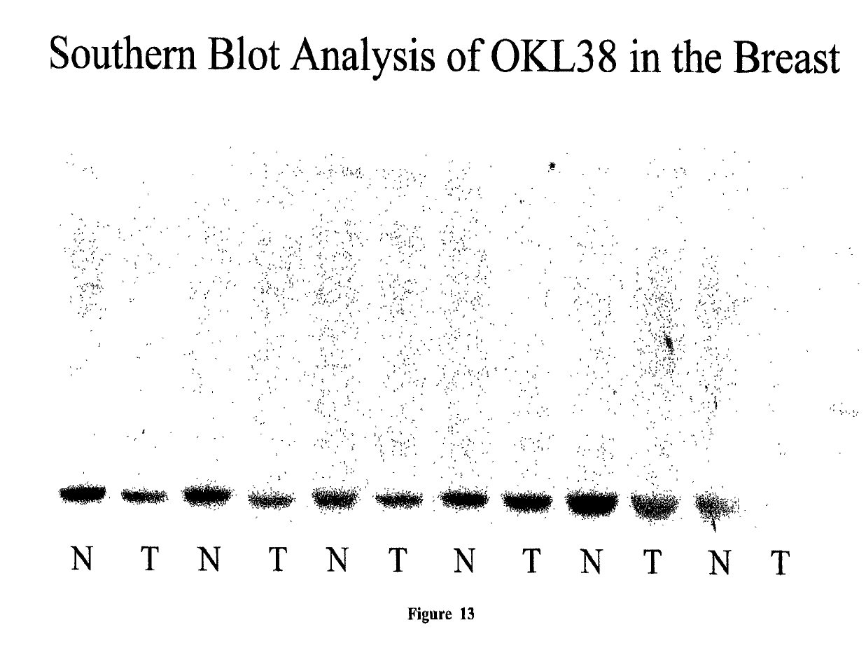

The results, shown in FIG. 13, suggest that there is a loss of one copy of the OKL38 gene in breast tumours.

### Example 15

The lit/lit mutation mouse model (31, 32) was taken advantage to examine the effects of growth hormone (GH) on OKL38 gene expression in the foetal liver. The lit/lit mutation results in a loss of function of pituitary GH releasing hormone receptors rendering the pituitary gland unable to respond to hypothalamic stimulation for GH production (32). As a consequence, these animals are deficient in GH and circulating insulin-like growth factor I (IGF-I), and underexpress genes whose expression is up-regulated by GH such as hepatic IGF-I. The lit/lit mouse (purchased from Jackson Laboratory) has intact GH and IGF-I genes, but pituitary GH expression, hepatic IGF-I expression, and serum IGF-I levels are less than 5% normal (33). Comparison of hepatic OKL38 gene expression in control Lit+/− versus lit/lit, lit/lit vs. lit/lit Tgh growth hormone transgenic mice provides data regarding the specific regulation of OKL38 gene expression during liver development and differentiation.

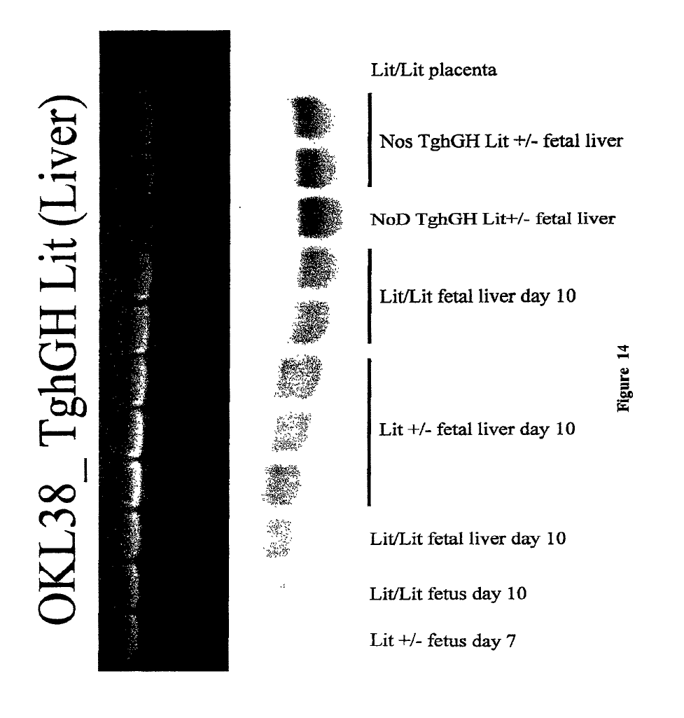

Total RNA from 7 day foetus Lit+/−, 10 day Lit/Lit foetus or 10 day foetal livers of Lit+/−, Lit/Lit and Tgh GH mice was isolated as previously described (34). Thirty μg total RNA was used for Northern blot analysis as previously described (34). The blots were hybridised with ³²P labelled OLK38 cDNA. Integrity and equal loading of RNA were verified by hybridising the blots to a human β-actin or GAPDH insert (35). The results, shown in FIG. 14, reveal that GH upregulates OKL38 in the liver.

In summary, the data presented herein indicate that OKL38 is a growth inhibitor and tumour suppressor. OKL38 expression is upregulated by pregnancy, GH and hormones that induce differentiation such as hCG. Drugs that block breast epithelial cell proliferation such as antiestrogens, taxol and doxorubicin also upregulate OKL38 gene expression. In all cases, the expression of OKL38 is associated with differentiation and low proliferative rate. The data suggest that enhancement of OKL38 production by cancer or tumour cells represent a new strategy for the arrest of cancer or tumour growth.

The disclosure of every patent, patent application, and publication cited herein is hereby incorporated herein by reference in its entirety.

The citation of any reference herein should not be construed as an admission that such reference is available as “Prior Art” to the instant application

Throughout the specification the aim has been to describe the preferred embodiments of the invention without limiting the invention to any one embodiment or specific collection of features. Those of skill in the art will therefore appreciate that, in light of the instant disclosure, various modifications and changes can be made in the particular embodiments exemplified without departing from the scope of the present invention. All such modifications and changes are intended to be included within the scope of the appended claims.

### BIBLIOGRAPHY

1. Miller, A. B. and Bulbrook, R. D. UICC multidisciplinary project on breast cancer: the epidemiology, aetiology and prevention of breast cancer. Int.J.Cancer, 37: 173, 1986.

2. Trichopoulos, D., Hsieh, C. C., MacMahon, B., Lin, T. M., Lowe, C. R., Mirra, A. P., Ravnihar, B., Salber, E. J., Valaoras, V. G., and Yuasa, S. Age at any birth and breast cancer risk. Int.J.Cancer, 31: 701-704, 1983

3. MacMahon, B., Cole, P., Liu, M., Lowe, C. R., Mirra, A. P., Ravinhar, H., Salber, E. J., Valaoras, V. G., and Yuasa, S. Age at birth and breast cancer risk. Bull WHO, 34: 209-221, 1970.

4. Russo, I. H. and Russo, J. Hormone prevention of mammary carcinogenesis: a new approach in anticancer research. Anticancer Res., 8: 1247-1262, 1988.

5. Russo, J. and Russo, I. H. Is differentiation the answer in breast cancer prevention?. Int.Res.Commun., 10: 935-945, 1982.

6. Russo, J. and Russo, I. H. Influence of differentiation and cell kinetics on the susceptility of the rat mammary gland to carcinogensis. Cancer Res., 40: 2677-2687, 1980.

7. Russo, J., Russo, I. H., Ireland, M., and Saby, J. Increased resistance of multiparous rat mammary gland to neoplastic transformation by 7,12-DMBA. Proc.Am.Assoc.Cancer Res., 18: 140, 1977

8. Dicksond, R. B. and Lippman, M. E. Growth factors in breast cancer. Endo. Rev., 16: 559-589, 1995.

9. Cullen, K. J. and Lippman, M. E. Estrogen regulation of protein synthesis and cell growth in human breast cancer. Vitamins and Hormones, 45: 127-172, 1989.

10. Knabbe, C., Lippman, M. E., Wakefield, L. M., Flanders, K. C. Kasid, A., Derynck, R., and Dickson, R. B. Evidence that transforming growth factor-b is a hormonally regulated negative growth factor in human breast cancer cells. Cell, 48: 417-428, 1987.

11. Huynh, H., Yang, X., and Pollak, M. Estradiol and antiestrogens regulate a growth inhibitory insulin-like growth factor binding protein 3 autocrine loop in human breast cancer cells. J.Biol.Chem., 271: 1016-1021, 1996

12. Oh, Y., Muller, H. L., Lamson, G., and Rosenfield, R. G. Insulin-like growth factor (IGF)-independent action of IGF-binding protein-3 in Hs578T human breast cancer cells. J.Biol.Chem., 268:14964-14971, 1993.

13. Oh, Y., Muller, H. L., Pham, H., and Rosenfield, R. G. Demonstration of receptors for insulin-like growth factor binding protein-3 on Hs578T human breast cancer cells. J.Bio.Chem., 268: 26045-26048, 1993.

14. Oh, Y., Mullers, H. L., Ng, L., and Rosenfield, R. G. Transforming growth factor-b-induced cell growth inhibition in human breast cancel cells is mediated through insulin-like growth factor-binding protein-3 action. J.Biol.Chem., 270:13589-13592, 1995.

15. Huynh, H., Larsson, C., Narod, S., and Pollak, M. Tumour suppressor activity of the gene encoding mammary-derived growth inhibitor. Cancer Res., 55:2225-2231, 1995.

16. Dickson, R. B., McManaway, M. E., and Lippman, M. E. Estrogen-induced factors of breast cancer cells partially replace estrogen to promote tumor growth. Science, 232: 1540, 1986.

17. Ethier, S. P. Growth factor synthesis and human breast cancer progression. [Review]. Journal.of.the.National.Cancer Institute., 87: 964-973, 1995.

18. Ausubel, E. F., Brent, R., Kingston, R. E., Moore, D. D., Seidman, J. G., Smith, J. A., and Struhl, K. Current Protocols in Molecular Biology. Current Protocols, 1993

19. Russo, I. H., Koszalka, M., and Russo, J. Human chorionic gonadotropin and rat mammary cancer prevention. J.Natl.Cancer Inst., 82: 1286-1289, 1990.

20. Welsch, C. W. Host factors affecting the growth of carcinogen-induced rat mammary carcinomas: A review and tribute to Charles Brenton Huggins. Cancer Res., 45:3415-3443, 1985.

21. Huynh, H., Beamer, W. D., and Pollak, M. Over-expression of insulin-like growth factor binding protein 4 (IGFBP-4) in MCF-7 breast cancer cells is associated with reduced responsiveness to insulin-like growth factors in vivo and reduced tumour growth in vivo. Int.J.Oncol., 11: 193-197, 1997.

22. Huynh, H., Tetenes, E., Wallace, L., and Pollak, M. In vivo inhibition of insulin-like growth factor-I gene expression by tamoxifen. Cancer Res., 53: 1727-1730, 1993.

23. Huang, H. S., Yee, J., Shew, J., Chen, P., Bookstein, R., Friedmann, T., Lee, E. Y. H. P., and Lee, W. Suppression of the neoplastic phenotype by replacement of the RB gene in human cancer cells. Science, 242: 1563-156, 1988.

24. Casey, G., Lo-Hsueh, M., Lopez, M. E., Vogelstein, B., and Stanbridge, E. J.

Growth suppression of human breast cancer cells by the introduction of a wild-type p53 gene. Oncogene, 6: 1791-1797, 1991.

25. Wang, N. P., To, H., Lee, W. H., and Lee, E. Y. H. P. Tumor suppressor activity of RB and p53 genes in human breast carcinoma cells. Oncogene, 8: 279-288, 1993.

26. Hao, Y., Crenshaw, T., Moulton, T., Newcomb, E., and Tycko, B. Tumor-suppressor activity of H19 RNA. Nature, 365: 764-767, 1993.

27. Knudson, A.g. Antioncogenes and human cancer. Proc.Natl.Acad.Sci.USA, 90: 10914-10921, 1993.

28. Ohtani-Fujita, N., Fujita, T., Aoike, A., Osifchin, N. E., Robbins, P. D., and Sakai, T. CpG methylation inactivates the promoter activity of the human retinoblastoma tumor-suppressor gene. Oncogene, 8(4): 1063-1067, 1993.

29. Huynh, H., Alpert, L., and Pollak, M. Silencing of the mammary derived growth inhibitor (MDGI) gene in breast neoplasms is associated with epigenetic changes. Cancer Res., 56: 4865-4870, 1996.

30. Grubbs, C. J., Farnell, D. R., Hill, D. L., and McDonough, K. C. Chemoprevention of N-nitroso-N-methylurea-induced mammary cancers by pretreatment with 17b-estradiol and progesterone. JNCI, 74: 927-931, 1985.

31. Eicher, E. and Beamer, W. Inherited ateliotic dwarfism in mice. Characteristics of the mutation, little, on chromosome 6. Journal of Heredity, 67: 87-91, 1976.

32. Godfrey, P., Rahal, J. O., Beamer, W. G., Copeland, N. G., Jenkins, N. A., and Mayo, K. E. GHRH receptor of little mice contains a missense mutation in the extracellular domain that disrupts receptor function. Nature Genet., 4: 227-232, 1993.

33. Yang, X., Beamer, W., Huynh, H. T., and Pollak, M. Reduced growth of human breast cancer xenografts in hosts homozygous for the ‘lit’ mutation. Cancer Res., 56: 1509-1511, 1996.

34. Huynh, H., Tetenes, E., Wallace, L., and Pollak, M. In vivo inhibition of insulin-like growth factor-I gene expression by tamoxifen. Cancer Res., 53: 1727-1730, 1993.

35. Adams, M. D., Kelley, J. M., Gocayne, J. D., Dubnick, M., Polymeroppulos, M. H., Xiao, H., Merril, C. R., Wu, A., Olde, B., Moreno, R. F., Kerlavage, A. R., McCombie, W. R., and Venter, J. C. Complementary DNA sequencing: expressed sequence tags and human genome project. Science, 252: 1651-1656, 1991.

36. Kuhlein, U., Gavora, J. S., Spencer, J. L., Bernon, D. E., and Sabour, M. Incidence of endogenous viral genes in two strains of White Leghorn chickens selected for egg production and susceptibility or resistance to Marek's disease. Theor.Appl.Genet., 77: 26-32, 1989.

37. Sambrook, J., Fritsch, E. F., and Maniatis, T. 655. Molecular Cloning: A laboratory manual, 2 ed, p. F.6-F.7. Cold Spring Harbor,N.Y.: Cold Spring Harbor Laboratory, 1989.

What is claimed is:

1. A composition for promoting, augmenting or otherwise enhancing cell differentiation, comprising an isolated polypeptide comprising the sequence set forth in SEQ ID NO 2.

2. A composition for delaying, repressing or otherwise inhibiting cell proliferation or tumorigenesis, comprising an isolated polypeptide comprising the sequence set forth in SEQ ID NO 2.

3. A biologically active fragment of a polypeptide comprising the sequence set forth in SEQ ID NO: 2, wherein said fragment is at least amino acids in length.

4. The fragment of claim 3 selected from the group consisting of residues 1-8, 9-16, 17-24, 25-32, 33-40, 41-48, 49-56, 57-64, 65-72, 73-80, 81-88, 89-96, 97-104, 105-112, 113-120, 121-128, 129-136, 137-144, 145-152, 153-160, 161-168, 169-176, 177-184, 185-192, 193-200, 201-208, 209-216, 217-224, 225-232, 223-240, 241-248, 249-256, 257-264, 265-272, 273-280, 281-288, 289-296, 297-304, 305-312 and 310-317 of SEQ ID NO: 2.

5. A composition for promoting, augmenting or otherwise enhancing cell differentiation, comprising an isolated polypeptide comprising an amino acid sequence having at least 80% identity to the sequence set forth in SEQ ID NO: 2.

6. The composition of claim 5, wherein the isolated polypeptide is distinguished from the sequence set forth in SEQ ID NO: 2 by substitution of at least one amino acid residue.

7. The composition of claim 6, wherein said substitution is a conservative substitution.

8. A composition for delaying, repressing or otherwise inhibiting cell proliferation or tumorigenesis, comprising an isolated polypeptide comprising an amino acid sequence having at least 80% identity to the sequence set forth in SEQ ID NO: 2.

9. The composition of claim 8, wherein the isolated polypeptide is distinguished from the sequence set forth in SEQ ID NO: 2 by substitution of at least one amino acid residue.

10. The composition of claim 9, wherein said substitution is a conservative substitution.

11. A composition for promoting, augmenting or otherwise enhancing cell differentiation, comprising an isolated polynucleotide comprising a nucleotide sequence encoding the sequence set forth in SEQ ID NO 2, or encoding a biologically active fragment of SEQ ID NO: 2, which is at least 8 amino acids in length.

12. A composition for delaying, repressing or otherwise inhibiting cell proliferation or tumorigenesis, comprising an isolated polynucleotide comprising a nucleotide sequence encoding the sequence set forth in SEQ ID NO 2, or encoding a biologically active fragment of SEQ ID NO: 2, which is at least 8 amino acids in length.

13. The composition of claim 11 or claim 12, wherein the isolated polynucleotide comprises the sequence set forth in any one of SEQ ID NO: 1, 3, and 5 or a fragment thereof at least 24 nucleotides in length.

14. The composition of claim 11 or claim 12, wherein the isolated polynucleotide comprises a sequence that hybridises under high stringency conditions to the sequence set forth in any one of SEQ ID NO: 1, 3, and 5 or to a fragment thereof at least 24 nucleotides in length.

15. An isolated polynucleotide comprising the sequence set forth in SEQ ID NO: 5, or a fragment thereof at least 24 nucleotides in length.

16. An isolated polynucleotide which hybridises under high stringency conditions to the sequence set forth in SEQ ID NO: 5, or a fragment thereof at least 24 nucleotides in length.

17. An isolated polynucleotide encoding a polypeptide which modulates an activity selected from the group consisting of cell differentiation, cell proliferation and tumorigenesis and which hybridises under high stringency conditions to the sequence set forth in SEQ ID NO: 5, or a fragment thereof at least 24 nucleotides in length.

18. A composition for promoting, augmenting or otherwise enhancing cell differentiation, comprising a vector including an isolated polynucleotide comprising a nucleotide sequence encoding the sequence set forth in SEQ ID NO 2, or encoding a biologically active fragment of SEQ ID NO: 2, which is at least 8 amino acids in length.

19. A composition for delaying, repressing or otherwise inhibiting cell proliferation or tumorigenesis, comprising a vector including an isolated polynucleotide comprising a nucleotide sequence encoding the sequence set forth in SEQ ID NO 2, or encoding a biologically active fragment of SEQ ID NO: 2, which is at least 8 amino acids in length.

20. A composition for promoting, augmenting or otherwise enhancing cell differentiation, comprising an expression vector including an isolated polynucleotide comprising a nucleotide sequence encoding the sequence set forth in SEQ ID NO 2, or encoding a biologically active fragment of SEQ ID NO: 2, which is at least 8 amino acids in length, wherein said polynucleotide is operably linked to a regulatory polynucleotide.

21. A composition for delaying, repressing or otherwise inhibiting cell proliferation or tumorigenesis, comprising an expression vector including an isolated polynucleotide comprising a nucleotide sequence encoding the sequence set forth in SEQ ID NO 2, or encoding a biologically active fragment of SEQ ID NO: 2, which is at least 8 amino acids in length, wherein said polynucleotide is operably linked to a regulatory polynucleotide.

22. A host cell containing an expression vector including an isolated polynucleotide comprising a nucleotide sequence encoding the sequence set forth in SEQ ID NO 2, or encoding a biologically active fragment of SEQ ID NO: 2, which is at least 8 amino acids in length, wherein said polynucleotide is operably linked to a regulatory polynucleotide..

23. A method of producing a recombinant polypeptide comprising the sequence set forth in SEQ ID NO 2, or biologically active fragment thereof at least 8 amino acids in length, comprising:
culturing a host cell containing the vector of claim 14 such that said recombinant polypeptide is expressed from said polynucleotide; and
isolating said recombinant polypeptide.

24. A method of screening for an agent which modulates at least one activity selected from the group consisting of cell differentiation, cell proliferation and tumorigenesis, said method comprising:
contacting a preparation comprising a member selected from the group consisting of the polypeptide sequence set forth in SEQ ID NO: 2, a biologically active fragment of said polypeptide, a variant of said polypeptide having at least 80% identity thereto, a variant of said fragment having at least 80% identity thereto, and a genetic sequence encoding said polypeptide, fragment or variant, with a test agent; and
detecting a change in the level and/or functional activity of said member or an expression product of said genetic sequence.

25. A method for detecting a specific polypeptide or polynucleotide sequence, comprising detecting a sequence of:
SEQ ID NO: 2 or 6, or a fragment thereof at least 8 amino acids residues in length; or
SEQ ID NO: 1 or 3, or a fragment thereof at least 24 nucleotides in length.

26. An antigen-binding molecule that is specifically immuno-interactive with a polypeptide comprising the sequence set forth SEQ ID NO: 2, or a fragment thereof at least 8 amino acids in length.

27. A method of detecting in a biological sample a polypeptide comprising the sequence set forth in SEQ ID NO: 2, comprising:
contacting the sample with the antigen-binding molecule of claim 23; and
detecting the presence of a complex comprising said antigen-binding molecule and said polypeptide in said contacted sample.

28. A method for detecting a polypeptide comprising the sequence set forth in any one of SEQ ID NO: 2, comprising:
detecting expression in a cell of a polynucleotide encoding said polypeptide.

29. A method for diagnosis of a cancer or tumour in a patient, comprising:
providing a biological sample from said patient; and
detecting relative to a normal reference value, a reduction in the level and/or functional activity of a member selected from the group consisting of a polypeptide comprising the sequence set forth in SEQ ID NO: 2, a variant thereof having at least 80% identity thereto, and a polynucleotide comprising the sequence set forth in SEQ ID NO: 1 or 3, or variant thereof.

30. The method of claim 29, wherein the cancer or tumour is associated with an organ selected from the group consisting of breast, liver, kidney, prostate, testis, bladder and lung.

31. The method of claim 29, wherein the cancer or tumour is of the breast.

32. A method for diagnosis of a cancer or tumour in a patient, comprising:
providing a biological sample from said patient; and
detecting an aberrant OKL38 polynucleotide or aberrant OKL38 polypeptide.

33. The method of claim 32, wherein the cancer or tumour is associated with an organ selected from the group consisting of breast, liver, kidney, prostate, testis, bladder and lung.

34. The method of claim 32, wherein the cancer or tumour is of the breast.

35. A method for diagnosis of a cancer or tumour in a patient, comprising:
providing a biological sample from said patient; and
detecting a loss of one or more copies of the OKL38 gene.

36. The method of claim 35, wherein the cancer or tumour is associated with an organ selected from the group consisting of breast, liver, kidney, prostate, testis, bladder and lung.

37. The method of claim 35, wherein the cancer or tumour is of the breast.

38. A method for diagnosis of a cancer or tumour in a patient, comprising:
contacting a biological sample obtained from said patient with the antigen-binding molecule of claim 26,
measuring the concentration of a complex comprising said antigen-binding molecule and a polypeptide comprising the sequence set forth in SEQ ID NO: 2, or variant thereof having at least 80% identity thereto, in said contacted sample; and
relating said measured complex concentration to the concentration of said polypeptide in said sample, wherein the presence of a reduced concentration relative to a normal reference concentration is indicative of said cancer or tumour.

39. The method of claim 38, wherein the cancer or tumour is associated with an organ selected from the group consisting of breast, liver, kidney, prostate, testis, bladder and lung.

40. The method of claim 28, wherein the cancer or tumour is of the breast.

41. A method for modulating an activity selected from the group consisting of cell differentiation, cell proliferation and tumorigenesis, comprising:
contacting a cell with an agent, which modulates the level and/or functional activity of a polypeptide comprising the sequence set forth in SEQ ID NO: 2, or a variant thereof having at least 80% identity thereto, for a time and under conditions sufficient to modulate the level and/or functional activity of said polypeptide.

42. A method for promoting, augmenting or otherwise enhancing cell differentiation, comprising:
contacting a cell with an agent, which increases the level and/or functional activity of a polypeptide comprising the sequence set forth in SEQ ID NO: 2, or a variant thereof having at least 80% identity thereto, for a time and under conditions sufficient to increase the level and/or functional activity of said polypeptide.

43. A method for delaying, repressing or otherwise inhibiting cell proliferation or tumorigenesis, comprising:
contacting a cell with an agent, which increases the level and/or functional activity of a polypeptide comprising the sequence set forth in SEQ ID NO: 2, or a variant thereof having at least 80% identity thereto, for a time and under conditions sufficient to increase the level and/or functional activity of said polypeptide.

44. The composition of any one of claims 1, 2, 5 and 8, comprising a pharmaceutically acceptable carrier.

45. A method for treatment and/or prophylaxis of a cancer or tumour, said method comprising administering to a patient in need of such treatment a therapeutically effective amount of an agent which modulates the level and/or functional activity of a polypeptide comprising the sequence set forth in any one of SEQ ID NO: 2, or a variant thereof having at least 80% identity thereto, for a time and under conditions sufficient to modulate the level and/or functional activity of said polypeptide.

46. The method of claim 45, wherein the cancer or tumour is associated with an organ selected from the group consisting of breast, liver, kidney, prostate, testis, bladder and lung.

47. The method of claim 45, wherein the cancer or tumour is of the breast.

48. A method for treatment and/or prophylaxis of a cancer or tumour, said method comprising administering to a patient in need of such treatment a therapeutically effective amount of a polypeptide comprising the sequence set forth in any one of SEQ ID NO: 2, or a variant thereof having at least 80% identity thereto.

49. The method of claim 48, wherein the cancer or tumour is associated with an organ selected from the group consisting of breast, liver, kidney, prostate, testis, bladder and lung.

50. The method of claim 48, wherein the cancer or tumour is of the breast.

51. A method for treatment and/or prophylaxis of a cancer or tumour, said method comprising administering to a patient in need of such treatment a therapeutically effective amount of a polynucleotide from which a polypeptide, or a variant thereof having at least 80% identity thereto, can be expressed, wherein said polypeptide comprises the sequence set forth in SEQ ID NO: 2.

52. The method of claim 51, wherein the cancer or tumour is associated with an organ selected from the group consisting of breast, liver, kidney, prostate, testis, bladder and lung.

53. The method of claim 51, wherein the cancer or tumour is of the breast.
